(19) United States
(12) Patent Application Publication
(10) Pub. No.: US 2026/0109740 A1
(43) Pub. Date: Apr. 23, 2026

(54) Polyamine Reporters

(71) Applicant: Whitehead Institute for Biomedical Research, Cambridge, MA (US)

(72) Inventors: Pushkal Sharma, Cambridge, MA (US); Ankur Jain, Cambridge, MA (US)

(21) Appl. No.: 19/307,589

(22) Filed: Aug. 22, 2025

Related U.S. Application Data
(60) Provisional application No. 63/686,522, filed on Aug. 23, 2024.

(51) Int. Cl. C07K 14/47; C12N 15/62; C12N 15/63; G01N 33/68

(52) U.S. Cl. CPC C07K 14/47; C12N 15/625; C12N 15/63; G01N 33/68; C07K 2319/09; C07K 2319/60; C07K 2319/95

(57) ABSTRACT

The disclosure provides, in various embodiments, polynucleotides comprising a polyamine-responsive element and a reporter protein coding sequence encoding a reporter protein, wherein interaction between a polyamine and the polyamine-responsive element modulates expression of the reporter protein. The disclosure also provides, in various embodiments, vectors cells, and kits comprising the polynucleotides, methods of reporting an intracellular polyamine, and methods of detecting and/or reporting an intracellular polyamine analog.

### RELATED APPLICATIONS

This application claims the benefit of U.S. Provisional Application No. 63/686,522, filed on Aug. 23, 2024. The entire teachings of the above application are incorporated herein by reference.

### GOVERNMENT SUPPORT

This invention was made with government support under grant number GM151111 awarded by the National Institutes of Health. The government has certain rights in the invention.

### INCORPORATION BY REFERENCE OF MATERIAL IN XML

This application incorporates by reference the Sequence Listing contained in the following eXtensible Markup Language (XML) file being submitted concurrently herewith: File name: 03992075-001_SL.xml; created: Aug. 20, 2025; 173,966 Bytes in size.

### BACKGROUND

Polyamines are organic cations that are essential for cell growth and survival. Abnormalities in polyamine transport are implicated in several devastating diseases, including Kufor-Rakeb syndrome (juvenile-onset parkinsonism with dementia) (Ramirez (2006)), early-onset Parkinson's disease (Lin (2008)), amyotrophic lateral sclerosis (ALS) (Spataro (2019)), and pulmonary arterial hypertension Gräf (2018)). Additionally, as rapidly proliferating cells exhibit high polyamine levels, there has been longstanding interest in developing polyamine inhibitors as anticancer therapeutics (Burns (2023), Gitto (2018)). Cancer cells can circumvent polyamine synthesis inhibition by increasing their uptake from the extracellular environment (Casero (2018)), and there is a growing interest in targeting the polyamine import pathway via combination therapies (Burns (2023), Gitto (2018)). Efforts to identify modulators of polyamine transport have been hampered by the lack of methods that provide a quantitative readout for polyamine uptake on a scale that is compatible with high-throughput screening.

### SUMMARY

There is a critical need to develop polyamine reporters that enable live-cell measurement, for example, at single-cell resolution. The disclosure provides such reporters.

Provided herein, among other things, are polynucleotides comprising:
a) a polyamine-responsive element, and
b) a reporter protein coding sequence encoding a reporter protein,
   wherein interaction between a polyamine and the polyamine-responsive element modulates expression of the reporter protein.

Also provided herein, among other things, are polynucleotides comprising, in 5′ to 3′ order:
a) a polyamine-responsive frameshifting element, and
b) a reporter protein coding sequence encoding a reporter protein, wherein:
   i. the reporter protein coding sequence lacks a start codon, and
   ii. expression of the reporter protein requires a polyamine-responsive frameshift.

Also provided herein, among other things, are polynucleotides comprising, in 5′ to 3′ order:
a) a control protein coding sequence encoding a control protein,
b) a polyamine-responsive frameshifting element, and
c) a reporter protein coding sequence encoding a reporter protein, wherein:
   i. the reporter protein coding sequence lacks a start codon, and
   ii. expression of the reporter protein requires a polyamine-responsive frameshift.

Also provided herein, among other things, are polynucleotides comprising, in 5′ to 3′ order:
a) an mCherry coding sequence encoding an mCherry,
b) a polyamine-responsive frameshifting element derived from a human OAZ1 gene, and
c) a reporter protein coding sequence encoding an eYFP or miRFP670-2, wherein the reporter protein coding sequence lacks an in-frame start codon,
   wherein interaction between a polyamine and the polyamine-responsive frameshifting element increases expression of the eYFP or miRFP670-2.

Also provided herein, among other things, are polynucleotides comprising, in 5′ to 3′ order:
an mCherry coding sequence encoding an mCherry,
a stop codon,
a polyamine-responsive frameshifting element derived from a human OAZ1 gene, and
a reporter protein coding sequence encoding an eYFP or miRFP670-2,
wherein interaction between a polyamine and the polyamine-responsive frameshifting element increases expression of the eYFP or miRFP670-2, and wherein
a) the reporter protein coding sequence lacks an in-frame start codon,
b) the mCherry coding sequence has an in-frame start codon, or both a) and b).

Also provided herein, among other things, are vectors comprising one or more of the polynucleotides disclosed herein.

Also provided herein, among other things, are cells comprising one or more of the polynucleotides or vectors disclosed herein.

Also provided herein, among other things, are kits comprising one or more of the polynucleotides, vectors, or cells disclosed herein.

Also provided herein, among other things, are methods of reporting an intracellular polyamine, comprising providing any one or more of the cells disclosed herein, and obtaining a measurement comprising:
a) an expression of the reporter protein in the cell, or a temporal change thereof,
b) a ratio of expression between the reporter protein and the control protein, or a temporal change thereof,
or both a) and b).

Also provided herein, among other things, are methods of detecting and/or reporting an intracellular polyamine analog, comprising providing any one or more of the cells disclosed herein, and obtaining a measurement comprising:
a) an expression of the reporter protein in the cell, or a temporal change thereof,
b) a ratio of expression between the reporter protein and the control protein, or a temporal change thereof,
or both a) and b).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1L Polyamine sensor design and validation. FIG. 1A Schematic of polyamine-responsive +1 ribosomal frameshifting during translation of OAZ1 mRNA. FIG. 1B illustrates a design for a polyamine sensor. FIGS. 1C-1E Immunoblot (1C), representative fluorescence micrographs (1D), and corresponding quantification of frameshift efficiency using flow-cytometry (1E) for cells expressing polyamine sensor under indicated treatments. DFMO: difluoromethylornithine, 1 mM; and Spd: spermidine, 5 μM. FIGS. 1F-1G Representative fluorescence micrographs (1F) and corresponding quantification by flow cytometry of cells (1G) upon genetic knockdown of indicated polyamine biosynthesis enzymes. FIG. 1H Single-cell traces of F/F₀ upon indicated treatments. F is the miRFP670-2 to mCherry fluorescence. F₀ is the average frameshifting efficiency in untreated cells (at the time, t=0). Arrows indicate the time-point of treatment. FIG. 1I Mean F/F₀ over time from the single-cell traces shown in 1H. Shaded bands depict the 95% confidence intervals. FIG. 1J Intracellular spermidine concentration measured using LC/MS (left) and F/F₀ (right) in response to various concentrations of DFMO. FIG. 1K Linear regression of the cellular spermidine concentration with F/F₀ (calculated from 1J). FIG. 1L F/F₀ and the spermidine concentration measured using LC/MS upon treatment with ribavirin (100 μM for 72 hr; SAT1 activator). The predicted value was calculated using the fit from 1K. Each data point shown in 1E, 1G, and 1L (left) represents a single cell. Error bars denote the median±interquartile range and were calculated from >10,000 cells. 50 data points are shown. Error bars in 1J (left) and 1L (right) denote the mean±standard deviation (n≥3 independent measurements). Flow cytometry quantification and the fluorescent micrographs are representative of ≥2 independent experiments. Significance values in 1E and 1G were calculated using Student's t-test. Scale bars, 10 μm.

FIG. 2A Chemical structures of mammalian polyamines. FIGS. 2B-2I Representative fluorescent micrographs (B, D, F, H) and corresponding quantification by flow cytometry (C, E, G, I) of cells expressing various OAZ1-derived candidate sequences under indicated treatments. The position of the nucleotide sequence in the endogenous OAZ1 mRNA transcript is labeled in blue, where the start codon is encoded by bases 1-3. Each data point represents a single cell. Error bars denote the median±interquartile range and are calculated from ≥5,000 cells. 50 data points are shown. Flow cytometry quantification and the fluorescent micrographs are representative of ≥2 independent experiments. Significance values were calculated using Student's t-test. Scale bars, 10 μm.

FIG. 3A Immunoblot of the indicated samples using an anti-ODC1 antibody. FIG. 3B Total polyamine levels (see “Enzymatic Total Polyamine Assay” in Example 1) for the indicated samples (in nmol per million cells). DFMO was used as a positive control. Significance values in (b) were calculated using Student's t-test. Error bars denote the mean±standard deviation (n=3).

FIGS. 4A-4B Representative fluorescence images (4A) and corresponding flow cytometry quantification (4B) of cells expressing either the polyamine sensor or its mutated version with a stop codon immediately after the mCherry coding region. FIG. 4C Sashimi plot for the sensor upon RNA sequencing, where the x-axis is the base coordinate within the sensor construct, and the y-axis indicates the number of reads per million mapped sequencing reads. Splicing events with at least 10 reads per junction, as detected by the STAR aligner, are indicated by arcs. Spliced sensor transcripts represent a negligible population (approximately 0.06%) of the total and would not significantly affect the F/F₀ ratio. WPRE: Woodchuck hepatitis virus Posttranscriptional Regulatory Element. FIGS. 4D-4E Representative fluorescence images (4D) and corresponding flow cytometry quantification (4E) of cells expressing a reporter construct containing a −1 ribosomal frameshift motif derived from the SARS CoV2 genome under indicated treatments. Each data point shown in 4B and 4E represents a single cell. Error bars denote the median±interquartile range and were calculated from ≥5,000 cells. 50 data points are shown. Flow cytometry quantification and the fluorescent micrographs are representative of ≥2 independent experiments. Significance values in 4B were calculated using Student's t-test. Scale bars, 10 μm.

FIG. 5 Representative fluorescence images of ODC1 or SRM knockdown (KD) cells after supplementation with spermidine. Scale bar, 10 μm.

FIGS. 6A-6D Representative fluorescence images and corresponding quantification of frameshift efficiency in HEK293T (6A), SH-SY5Y (6B), RPE1 cells (6C), and primary murine intestinal organoids (6D) expressing sensor under indicated treatments. Two distinct populations of cells in intestinal organoids were observed, out of which, only one population responded to DFMO treatment and subsequent spermidine supplementation. Another population retained a low F/F₀ value throughout suggesting low de novo polyamine biosynthesis and import. Each data point represents a single cell. Error bars denote the median±interquartile range and were calculated from ≥5,000 cells. 50 data points (cells) are shown for representation purposes. Flow cytometry quantification and the fluorescent micrographs are representative of ≥2 independent experiments. FIG. 6E Flow-cytometry quantification of eYFP and mCherry for organoids in 6D. 500 data points are shown. Significance values in (6A)-(6C) were calculated using Student's t-test. Significance values in (6D) were calculated using the Mann-Whitney non-parametric test. Scale bars, 10 μm (except for intestinal organoids, 50 μm).

FIG. 7A Schematic of a sensor design for real-time tracking of polyamines. FIG. 7B Representative fluorescence images of cells expressing the degron-based sensor under indicated treatments. DFMO (2 mM), Spd (spermidine, 100 μM) and TMP (0.05 μM). Cells were grown in TMP starting 3 days before the beginning of the experiment to allow steady-state levels of the reporter. Media was replaced every two days with fresh reagents. FIGS. 7C-7D Cell-to-cell heterogeneity in response to DFMO treatment and subsequent spermidine rescue: For example, a cell whose polyamine levels are unresponsive to DFMO treatment (7C) and a cell where the maximal spermidine uptake is delayed by 60 hr (7D).

FIG. 8A Schematic of LC/MS protocol for absolute polyamine quantification. FIG. 8B Linear regression of the cellular polyamine concentration with F/F₀ (calculated from DFMO titration). FIG. 8C Intracellular spermidine concentration measured using LC/MS (yellow) and F/F₀ (grey) in response to DFMO and sardomozide titration. FIG. 8D Intracellular putrescine (purple) and spermine (blue) concentration measured using LC/MS in response to DFMO and sardomozide titration. Error bars denote the mean±standard deviation (n≥3 independent experiments).

FIG. 9A Quantification of frameshift efficiency using flow-cytometry in cells expressing polyamine sensor in three independent experiments over three days (nine independent experiments). F₀ is the ratio of eYFP to mCherry fluorescence in cells on Day 1 (replicate 1). All replicates are plotted on the graph. Error bars denote the median±interquartile range and were calculated from ≥5,000 cells. 50 data points are shown. FIG. 9B Coefficient of variation (CV) was calculated for intra-assay and inter-day variability. CV was calculated as the ratio of standard deviation to the average value.

FIGS. 10A-10J Modifiers of polyamine import. FIG. 10A Schematic of single-cell polyamine import assay. FIG. 10B Representative fluorescence images of cells upon indicated treatments. AMXT-1501 (0.5 μM; polyamine import inhibitor), DFMO (1 mM), and spermidine (5 μM). FIG. 10C −log 10 (MaGeCK score) versus median log 2 (fold enrichment) for all genes from the spermidine import screen. FIG. 10D Graph depicting the fold enrichment for sgRNAs targeting indicated genes. Each data point is for a distinct sgRNA sequence. FIG. 10E Heatmap of enrichment scores for individual sgRNAs targeting genes previously proposed to be involved in polyamine import. FIGS. 10F-10I Comparison of polyamine levels in wild-type and upon ATP13A3 knock-out in K562 (10F), U-2OS (10G), and RPE1 (10I) cells under indicated treatments. Representative images of U-2OS cells corresponding to treatment in (10H). DFMO: 1 mM and spermidine: 5 μM. FIG. 10J Comparison of polyamine levels in U-2OS cells under indicated treatments. Rotenone (1 μM; complex I inhibitor) and antimycin (1 μM; complex III inhibitor) were added 24 hr before reporter induction and spermidine addition (5 μM). Error bars denote the median±interquartile range and were calculated from ≥5,000 cells. 50 data points are shown. Flow cytometry quantification and the fluorescent micrographs are representative of ≥2 independent experiments. Significance values were calculated using Student's t-test. Scale bars, 10 μm.

FIG. 11A Quantification of frameshift efficiency using flow-cytometry for treatments in FIG. 10B. FIG. 11B Schematic of a CRISPR-Cas9 FACS-based genome-wide screen to identify modulators of polyamine import. FIG. 11C DESeq2 normalized read counts from RNA-sequencing of K562 cells. FIG. 11D Representative fluorescence micrographs of cells in FIG. 10G. FIGS. 11E-11F The mean fold-change in enrichment (bottom 30% vs. unsorted population based on eYFP/mCherry) from two technical replicates of the genome-wide screen (Area 1 versus Area 2). Only genes with an annotated transmembrane transporter activity are shown in 11E after excluding mitochondrial (MitoCarta3.0) genes, common essential genes (Hart (2015)) for which >2 sgRNAs were detected. FIG. 11G Barplot of −log 10(FDR) for gene sets (WikiPathways GSEA enrichment) with positive FDR<0.05 (requiring >2 sgRNAs/gene) with abbreviated gene set names. FIG. 11H Total polyamine levels (see “Enzymatic Total Polyamine Assay” in Example 1) for the indicated samples (relative abundance). Rotenone (1 μM), antimycin (1 μM), and spermidine (5 μM). 2 mM DFMO (48 hr) was used as a positive control. Error bars in 11A denote the median±interquartile range and were calculated from ≥5,000 cells. 50 data points are shown. Flow cytometry quantification and the fluorescent micrographs represent ≥2 independent experiments. Error bars in 11C denote the mean±standard deviation from 3 independent experiments. Significance values in 11H were calculated using Student's t-test. Scale bars, 10 μm.

FIG. 12 A map of a non-limiting example of a polyamine reporter construct.

### DETAILED DESCRIPTION

A description of example embodiments follows.

### Definitions

Certain terms used herein is for the purpose of describing particular embodiments only and is not intended to be limiting.

As used herein, the term “a,” “an,” or “the” should be understood to include plural reference unless the context clearly indicates otherwise.

As used herein, unless the context requires otherwise, the term “comprise,” and variations such as “comprises” and “comprising”, will be understood to imply the inclusion of, e.g., a stated integer or step or group of integers or steps, but not the exclusion of any other integer or step or group of integer or step. As used herein, the term “comprising” can be substituted with the term “containing” or “including.”

As used herein, “consisting of” excludes any element, step, or ingredient not specified in the claim element. As used herein, “consisting essentially of” does not exclude materials or steps that do not materially affect the basic and novel characteristics of the claim. Any of the terms “comprising,” “containing,” “including,” and “having,” whenever used herein in the context of an aspect or embodiment of the disclosure, can in some embodiments, be replaced with the term “consisting of,” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and, therefore, satisfy the requirement of the term “and/or” as used herein. Concurrent applicability of more than one of the options is also understood to fall within the meaning, and, therefore, satisfy the requirement of the term “and/or.”

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

Unless otherwise indicated or otherwise evident from the context and/or understanding of one of ordinary skill in the art, values herein that are expressed as ranges can assume any specific value or sub-range within the stated ranges in different embodiments disclosed herein, to the tenth of the unit of the lower limit of the range, unless the context clearly dictates otherwise.

As used herein, the term “about” means within an acceptable error range for a particular value, as determined by one of ordinary skill in the art. Typically, an acceptable error range for a particular value depends, at least in part, on how the value is measured or determined, e.g., the limitations of the measurement system. For example, “about” can mean within an acceptable standard deviation, per the practice in the art. It is to be understood that the term “about” can precede any particular value specified herein, except for particular values used in the Exemplification. When “about” precedes a range, as in “1-20”, the term “about” should be read as applying to both given values of the range, such that “about 1-20” means about 1 to about 20.

It should be understood that for all numerical bounds describing some parameter in this application, such as “about,” “at least,” “less than,” “fewer than,” and “more than,” the description also necessarily encompasses any range bounded by the recited values. Accordingly, for example, the description “at least 1, 2, 3, 4, or 5” also describes, inter alia, the ranges 1-2, 1-3, 1-4, 1-5, 2-3, 2-4, 2-5, 3-4, 3-5, and 4-5, etcetera.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “polynucleotide” refers to a biopolymer comprising naturally occurring deoxyribonucleotide monomers, non-naturally occurring deoxyribonucleotide monomers (e.g., 7-deazaguanosine, inosine, or a methylated nucleotide such as 5-methyl dCTP or 5-hydroxymethyl cytosine), naturally occurring ribonucleotide monomers, or non-naturally occurring ribonucleotide monomers (e.g., a locked nucleic acid (LNA)), or a combination thereof. A polynucleotide described herein can be single stranded (ss) or double stranded (ds). In some embodiments, a polynucleotide described herein is a DNA molecule. In some embodiments, a polynucleotide described herein is an RNA molecule (e.g., a linear or a circular RNA molecule).

The term “encoding” refers to specific sequences of nucleotides in a polynucleotide, such as a DNA (e.g., a cDNA) or an RNA (e.g., an mRNA), that serve as a template for synthesis of a protein having a defined sequence of amino acids. Unless otherwise specified, a polynucleotide encoding an amino acid sequence can have any one nucleic acid sequence of all nucleic acid sequences that are degenerate versions of each other and that encode the amino acid sequence.

The term “vector” refers to a nucleic acid molecule which may be employed to introduce a nucleic acid sequence or gene into a cell, either in vitro, ex vivo, or in vivo.

The term “ex vivo” refers to methods conducted within or on cells or tissue in an artificial environment outside an organism with minimum alteration of natural conditions.

The term “in vivo” refers to a method that is conducted within living organisms in their normal, intact state.

The term “in vitro” method is conducted using components of an organism that have been isolated from its usual biological context.

The term “expression vector” refers to a replicable nucleic acid from which one or more proteins can be expressed when the expression vector is transformed into a suitable expression host cell.

The term “host” cell refers to a cell into which a polynucleotide has been introduced by molecular biology techniques. All techniques by which a polynucleotide can be introduced into a host cell, including transfection with viral vectors, transformation with plasmid vectors, and introduction of naked DNA by electroporation, lipofection, and particle gun acceleration are contemplated herein.

The term “promoter” refers to a region of DNA to which RNA polymerase binds and initiates the transcription of a gene.

The term “operably linked” means that the nucleic acid is positioned in the recombinant polynucleotide, e.g., vector, in such a way that enables expression of the nucleic acid under control of the element (e.g., promoter) to which it is linked.

The term “selectable marker element” is an element that confers a trait suitable for artificial selection. Selectable marker elements can be negative or positive selection markers.

### Polynucleotide Reporters

Provided herein, among other things, is a polynucleotide comprising:
a) a polyamine-responsive element, and
b) a reporter protein coding sequence encoding a reporter protein,
wherein interaction between a polyamine and the polyamine-responsive element modulates expression of the reporter protein.

In some embodiments, interaction between a polyamine and a polyamine-responsive element increases expression of a reporter protein.

In some embodiments, interaction between a polyamine and a polyamine-responsive element decreases expression of a reporter protein.

Also provided herein, among other things, is a polynucleotide comprising (e.g., in 5′ to 3′ order):
a) a polyamine-responsive frameshifting element, and
b) a reporter protein coding sequence encoding a reporter protein, wherein:
   i. the reporter protein coding sequence lacks a start codon, and
   ii. a polyamine-responsive frameshift increases expression of the reporter protein.

Also provided herein, among other things, is a polynucleotide comprising (e.g., in 5′ to 3′ order):
a) a control protein coding sequence,
b) a stop codon, wherein the control protein coding sequence and the stop codon are in the same contiguous open reading frame,
c) a polyamine-responsive frameshifting element, and
d) a reporter protein coding sequence.

Also provided herein, among other things, is a polynucleotide comprising (e.g., in 5′ to 3′ order):
a) an mCherry coding sequence encoding an mCherry,
b) a stop codon, wherein the mCherry coding sequence and the stop codon are in the same contiguous open reading frame,
c) a polyamine-responsive frameshifting element derived from a human ornithine decarboxylase antizyme 1 (OAZ1) gene, and
d) a reporter protein coding sequence, wherein:
   i. the reporter protein coding sequence lacks an in-frame start codon,
   ii. expression of the reporter protein requires a polyamine-responsive +1 ribosomal frameshift, and
   iii. the reporter protein coding sequence encoding an enhanced yellow fluorescent protein (eYFP).

Also provided herein, among other things, is a polynucleotide comprising (e.g., in 5′ to 3′ order):
a) an mCherry coding sequence encoding an mCherry,
b) a stop codon, wherein the mCherry coding sequence and the stop codon are in the same contiguous open reading frame,
c) a polyamine-responsive frameshifting element derived from a human ornithine decarboxylase antizyme 1 (OAZ1) gene, and
d) a reporter protein coding sequence, wherein:
   i. the reporter protein coding sequence lacks an in-frame start codon,
   ii. expression of the reporter protein requires a polyamine-responsive +1 ribosomal frameshift, and
   iii. the reporter protein coding sequence encoding miRFP670-2z.

### Polynucleotides

In some embodiments, a polynucleotide is single stranded (ss).

In some embodiments, a polynucleotide is double stranded (ds).

In some embodiments, a polynucleotide is a DNA molecule (e.g., a linear or a circular DNA molecule).

In some embodiments, a polynucleotide is integrated into the genomic DNA.

In some embodiments, a polynucleotide is extrachromosomal.

In some embodiments, a polynucleotide is an RNA molecule (e.g., a linear or a circular RNA molecule).

In some embodiments, a polynucleotide is an mRNA.

In some embodiments, an mRNA is in vitro transcribed (e.g., using a kit such as the MEGAscript™ T7 Transcription Kit (Invitrogen™ AM1334)) from a DNA template. In some embodiments, an in vitro transcribed mRNA is modified to increase stability and/or to reduce immunogenicity, for example, by adding a 5′ cap and/or substituting one or more nucleotides (e.g., substituting a UTP solution with N1-Methylpseudouridine-5′-Triphosphate). For additional information on in vitro transcription and mRNA production, see, e.g., Neugebauer et al., Evolution of an adenine base editor into a small, efficient cytosine base editor with low off-target activity, Nat Biotechnol. 41(5):673-85 (2023), the entire contents of which are incorporated herein by reference.

In some embodiments, a polynucleotide comprises (e.g., in 5′ to 3′ order):
a control protein coding sequence encoding a control protein,
a polyamine-responsive frameshifting element, and
a reporter protein coding sequence encoding a reporter protein,
wherein interaction between a polyamine and the polyamine-responsive frameshifting element increases expression of the reporter protein,
optionally wherein:
a) the reporter protein coding sequence lacks an in-frame start codon,
b) the control protein coding sequence has an in-frame start codon, or
both a) and b).

In some embodiments, a polynucleotide comprises (e.g., in 5′ to 3′ order):
a polyamine-responsive frameshifting element,
a reporter protein coding sequence encoding a reporter protein, and
a control protein coding sequence encoding a control protein,
wherein interaction between a polyamine and the polyamine-responsive frameshifting element increases expression of the reporter protein,
optionally wherein:
a) the reporter protein coding sequence lacks an in-frame start codon,
b) the control protein coding sequence has an in-frame start codon, or both a) and b).

In some embodiments, a polynucleotide comprises (e.g., in 5′ to 3′ order):
a control protein coding sequence encoding a control protein,
a stop codon,
a polyamine-responsive frameshifting element, and
a reporter protein coding sequence encoding a reporter protein,
wherein interaction between a polyamine and the polyamine-responsive frameshifting element increases expression of the reporter protein,
optionally wherein:
a) the reporter protein coding sequence lacks an in-frame start codon,
b) the control protein coding sequence has an in-frame start codon, or both a) and b).

In some embodiments, a polynucleotide comprises (e.g., in 5′ to 3′ order):
a stop codon,
a polyamine-responsive frameshifting element,
a reporter protein coding sequence encoding a reporter protein, and
a control protein coding sequence encoding a control protein,
wherein interaction between a polyamine and the polyamine-responsive frameshifting element increases expression of the reporter protein,
optionally wherein:
a) the reporter protein coding sequence lacks an in-frame start codon,
b) the control protein coding sequence has an in-frame start codon, or
both a) and b).

In some embodiments, expression of a reporter protein requires a polyamine-responsive +1 ribosomal frameshift.

In some embodiments, a polynucleotide comprises (e.g., in 5′ to 3′ order):
an mCherry coding sequence encoding an mCherry,
a polyamine-responsive frameshifting element derived from a human ornithine decarboxylase antizyme 1 (OAZ1) gene, and
a reporter protein coding sequence encoding an eYFP or miRFP670-2,
wherein interaction between a polyamine and the polyamine-responsive frameshifting element increases expression of the eYFP or miRFP670-2,
optionally wherein:
a) the reporter protein coding sequence lacks an in-frame start codon,
b) the mCherry coding sequence has an in-frame start codon, or
both a) and b).

In some embodiments, a polynucleotide comprises (e.g., in 5′ to 3′ order):
a polyamine-responsive frameshifting element derived from a human OAZ1 gene,
a reporter protein coding sequence encoding an eYFP or miRFP670-2, and
an mCherry coding sequence encoding an mCherry,
wherein interaction between a polyamine and the polyamine-responsive frameshifting element increases expression of the eYFP or miRFP670-2, optionally wherein:
a) the reporter protein coding sequence lacks an in-frame start codon,
b) the mCherry coding sequence has an in-frame start codon, or
both a) and b).

In some embodiments, a polynucleotide comprises (e.g., in 5′ to 3′ order):
an mCherry coding sequence encoding an mCherry,
a stop codon,
a polyamine-responsive frameshifting element derived from a human OAZ1 gene, and
a reporter protein coding sequence encoding an eYFP or miRFP670-2,
wherein interaction between a polyamine and the polyamine-responsive frameshifting element increases expression of the eYFP or miRFP670-2,
optionally wherein:
a) the reporter protein coding sequence lacks an in-frame start codon,
b) the mCherry coding sequence has an in-frame start codon, or
both a) and b).

In some embodiments, a polynucleotide comprises (e.g., in 5′ to 3′ order):
a stop codon,
a polyamine-responsive frameshifting element derived from a human OAZ1 gene,
a reporter protein coding sequence encoding an eYFP or miRFP670-2, and
an mCherry coding sequence encoding an mCherry,
wherein interaction between a polyamine and the polyamine-responsive frameshifting element increases expression of the eYFP or miRFP670-2,
optionally wherein:
a) the reporter protein coding sequence lacks an in-frame start codon,
b) the mCherry coding sequence has an in-frame start codon, or
both a) and b).

In some embodiments, expression of an eYFP or miRFP670-2 requires a polyamine-responsive +1 ribosomal frameshift.

In some embodiments, a polynucleotide further comprises an inducible promoter operably linked to the reporter protein coding sequence. In some embodiments, an inducible promoter comprises or is a doxycycline-inducible promoter. In some embodiments, an inducible promoter comprises or is a tetracycline (Tet)-inducible promoter.

In some embodiments, a polynucleotide comprises a nucleotide sequence that is codon-optimized for a chosen host cell (e.g., a human cell).

### Polyamine-Responsive Elements

In some embodiments, a polyamine-responsive element is a polyamine-responsive frameshifting element.

In some embodiments, a polyamine-responsive frameshifting element comprises a shift site sequence (also called a slippery sequence or a recording site). In some embodiments, a shift site sequence comprises a cognate or near-cognate codon. In some embodiments, a shift site sequence comprises a cognate codon. In some embodiments, a shift site sequence comprises, consists essentially of, or consists of TCCTGA. In some embodiments, a shift site sequence comprises a near-cognate codon.

In some embodiments, a reading frame is shifted upstream. In some embodiments, a reading frame is shifted downstream.

In some embodiments, a polyamine-responsive frameshift is a +1 ribosomal frameshift or a −1 ribosomal frameshift (i.e., a +2 ribosomal frameshift). In some embodiments, a polyamine-responsive frameshift is a +1 ribosomal frameshift. In some embodiments, a polyamine-responsive frameshift is a −1 ribosomal frameshift.

In some embodiments, a polyamine-responsive frameshifting element increases frameshift efficiency between about 40 μM and about 4 mM spermidine. In some embodiments, a polyamine-responsive frameshifting element monotonically increases frameshift efficiency between about 40 μM and about 4 mM spermidine.

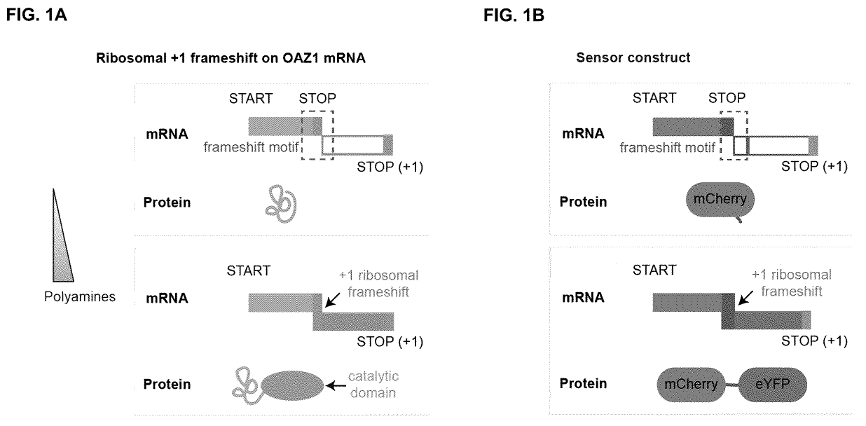

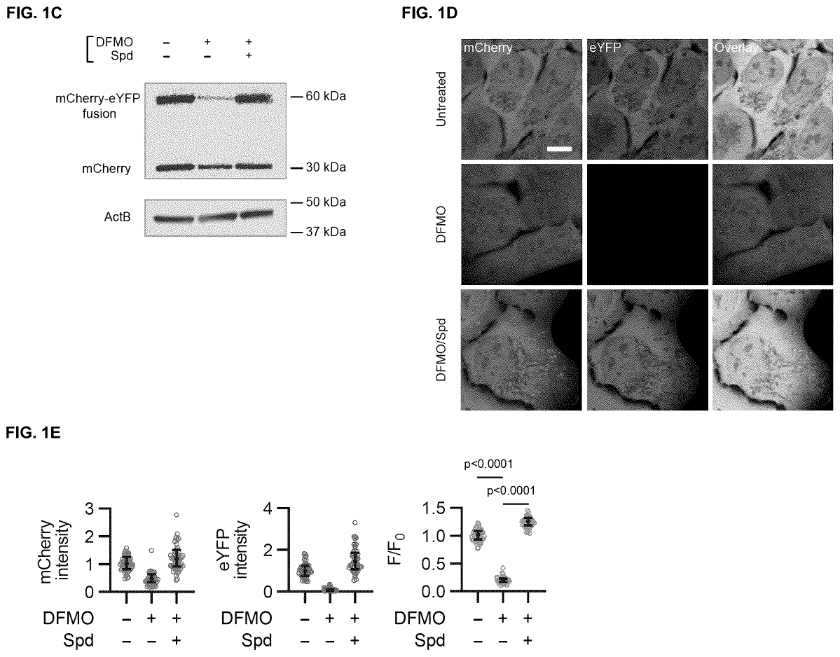

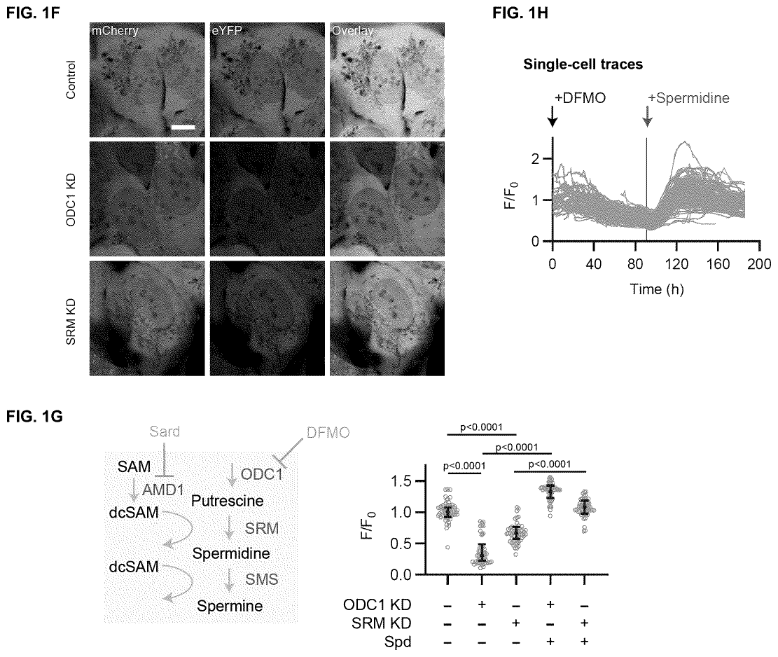

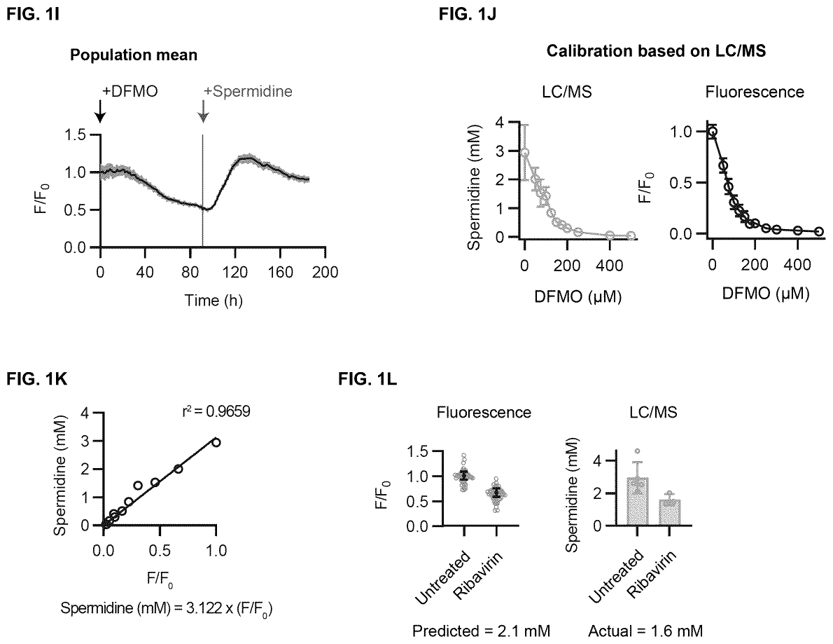

In some embodiments, a polyamine-responsive element further comprises a stimulator sequence (e.g., producing a strong RNA secondary structure) at the 3′ of a shift site sequence. In some embodiments, a polyamine-responsive element comprises a pseudoknot sequence at the 3′ of a shift site sequence. Non-limiting examples of pseudoknot sequences can be found in Ivanov et al., Conservation of polyamine regulation by translational frameshifting from yeast to mammals, EMBO J. 19(8):1907-17 (2000) (see, e.g., FIG. 1B), the contents of which are incorporated by reference herein in their entirety.

In some embodiments, a polyamine-responsive element further comprises a stimulator sequence at the 5′ of a shift site sequence.

In some embodiments, a stimulator sequence increases the efficiency of frameshift. In some embodiments, a stimulator sequence increases the efficiency of frameshift by at least 2 fold, for example, by at least: 3, 4, 5, 6, 7, 8, 9, 10, 25, 50, 100, 250, 500, 750, 1,000, 1,500, 2,000, 3,000, 5,000 or 10,000 fold. In some embodiments, a stimulator sequence increases the efficiency of frameshift by at least 100 fold. In some embodiments, a stimulator sequence increases the efficiency of frameshift by 2-10,000 fold, for example, 2-5,000, 3-5,000, 3-3,000, 4-3,000, 4-2,000, 5-2,000, 5-1,500, 6-1,500, 6-1,000, 7-1,000, 7-750, 8-750, 8-500, 9-500, 9-250, 10-250, or 10-100 fold.

In some embodiments, a polyamine-responsive frameshifting element is derived from an ornithine decarboxylase antizyme gene, for example, of Homo sapiens, Mus musculus, Gallus gallus, Xenopus laevis, Danio rerio, Drosophila melanogaster, Onchocerca volvulus, Pristioncus pacificus, Necator americanus, Haemonchus contortus, Caenorhabditis elegans, or Schizosaccharomyces pombe. In some embodiments, an ornithine decarboxylase antizyme gene is a human ornithine decarboxylase antizyme gene.

In some embodiments, a polyamine-responsive frameshifting element is derived from an ornithine decarboxylase antizyme 1 gene (e.g., a human OAZ1 gene such as NM_004152.3), an ornithine decarboxylase antizyme 2 gene (e.g., a human OAZ2 gene such as NM_002537.3), or an ornithine decarboxylase antizyme 3 gene (e.g., a human OAZ3 gene such as NM_016178.2).

In some embodiments, a polyamine-responsive frameshifting element is derived from an ornithine decarboxylase antizyme 3 gene. In some embodiments, an ornithine decarboxylase antizyme 3 gene is a human ornithine decarboxylase antizyme 3 gene.

In some embodiments, a polyamine-responsive frameshifting element is derived from an ornithine decarboxylase antizyme 2 gene. In some embodiments, an ornithine decarboxylase antizyme 2 gene is a human ornithine decarboxylase antizyme 2 gene.

In some embodiments, a polyamine-responsive frameshifting element is derived from an ornithine decarboxylase antizyme 1 gene (e.g., a human OAZ1 gene). In some embodiments, an ornithine decarboxylase antizyme 1 gene is a human ornithine decarboxylase antizyme 1 gene (e.g., SEQ ID NO:1).

In some embodiments, a polyamine-responsive frameshifting element derived from a human OAZ1 gene:
a) comprises TCCTGA and SEQ ID NO:2, SEQ ID NO:3, SEQ ID NO:4, SEQ ID NO:5, or SEQ ID NO:6,
b) lacks a catalytically active domain,
or both a) and b).

In some embodiments, a polyamine-responsive frameshifting element comprises an in-frame stop codon. In some embodiments, a polyamine-responsive frameshifting element comprises TCCTGA. In some embodiments, a polyamine-responsive frameshifting element comprises SEQ ID NO:2. In some embodiments, a polyamine-responsive frameshifting element comprises TCCTGA and SEQ ID NO:2 (e.g., at the 3′ of TCCTGA).

In some embodiments, a polyamine-responsive frameshifting element comprises (e.g., consists of) SEQ ID NO:3, SEQ ID NO:4, SEQ ID NO:5, or SEQ ID NO:6. In some embodiments, a polyamine-responsive frameshifting element comprises (e.g., consists of) SEQ ID NO:3. In some embodiments, a polyamine-responsive frameshifting element comprises (e.g., consists of) SEQ ID NO:4. In some embodiments, a polyamine-responsive frameshifting element comprises (e.g., consists of) SEQ ID NO:5. In some embodiments, a polyamine-responsive frameshifting element comprises (e.g., consists of) SEQ ID NO:6.

In some embodiments, a polyamine-responsive frameshifting element lacks a catalytically active domain.

In some embodiments, a polyamine-responsive frameshifting element:
a) comprises TCCTGA and SEQ ID NO:2, and
b) lacks a catalytically active domain.

In some embodiments, a polyamine-responsive frameshifting element:
a) comprises SEQ ID NO:3, and
b) lacks a catalytically active domain.

In some embodiments, a polyamine-responsive frameshifting element:
a) comprises SEQ ID NO:4, and
b) lacks a catalytically active domain.

In some embodiments, a polyamine-responsive frameshifting element:
a) comprises SEQ ID NO:5, and
b) lacks a catalytically active domain.

In some embodiments, a polyamine-responsive frameshifting element:
a) comprises SEQ ID NO:6, and
b) lacks a catalytically active domain.

It will be appreciated that “T” may be replaced by “U” in a polynucleotide sequence presented herein, e.g., a polynucleotide comprising or consisting of a polyamine-responsive element (e.g., a polyamine-responsive frameshifting element), e.g., when such polynucleotide is in the form of RNA (e.g., mRNA). For example, in some embodiments, a polyamine-responsive frameshifting element comprises UCCUGA. In some embodiments, a polyamine-responsive frameshifting element comprises UCCUGA and SEQ ID NO: 2, SEQ ID NO:3, SEQ ID NO:4, SEQ ID NO:5, or SEQ ID NO:6 (with T replaced by U in SEQ ID NO: 2, 3, 4, 5, or 6).

### Reporter Protein Coding Sequences

In some embodiments, interaction between a polyamine and a polyamine-responsive element (e.g., a polyamine-responsive frameshifting element) modulates (e.g., increases) expression of a reporter protein. In some embodiments, expression of a reporter protein requires interaction between a polyamine and a polyamine-responsive element (e.g., a polyamine-responsive frameshifting element).

In some embodiments, a polyamine-responsive element is a polyamine-responsive frameshifting element. In some embodiments, expression of a reporter protein requires ribosomes to shift from a first open reading frame to a second open reading frame (e.g., +1 or −1 open reading frame). In some embodiments, expression of a reporter protein requires ribosomes to shift from a first open reading frame to a second, +1 open reading frame. In some embodiments, expression of a reporter protein requires ribosomes to shift from a first open reading frame to a second, −1 open reading frame. In some embodiments, a reporter protein coding sequence lacks a start codon in the first open reading frame. In some embodiments, a reporter protein coding sequence lacks any in-frame methionine codon.

In some embodiments, interaction between a polyamine and a polyamine-responsive element (e.g., a polyamine-responsive frameshifting element) modulates (e.g., increases) expression of a reporter protein by at least 10 fold, for example, by at least: 25, 50, 100, 250, 500, 750, 1,000, 1,500, 2,000, 3,000, 5,000 or 10,000 fold. In some embodiments, interaction between a polyamine and a polyamine-responsive element (e.g., a polyamine-responsive frameshifting element) modulates (e.g., increases) expression of a reporter protein by at least 1,000 fold.

In some embodiments, interaction between a polyamine and a polyamine-responsive element (e.g., a polyamine-responsive frameshifting element) modulates (e.g., increases) expression of a reporter protein by about: 25-10,000 fold, for example, about: 25-5,000, 50-5,000, 50-3,000, 100-3,000, 100-2,000, 250-2,000, 250-1,500, 500-1,500, 500-1,000, or 750-1,000 fold.

In some embodiments, a reporter protein encoding sequence comprises an in-frame termination codon (e.g., stop-1). In some embodiments, a reporter protein encoding sequence comprises an in-frame termination codon located shortly after an initiation codon, for example, about: 1-500, 5-450, 5-400, 10-400, 10-350, 20-350, 20-300, 50-300, 50-250, 100-250, 100-200, or 150-200 nucleotides after an initiation codon.

In some embodiments, expression of a reporter protein requires interaction between a polyamine and a polyamine-responsive frameshifting element (e.g., a polyamine-responsive frameshifting element).

In some embodiments, interaction between a polyamine and a polyamine-responsive frameshifting element results in a +1 ribosomal frameshift, and a reporter protein coding sequence is in the +1-frame of the polyamine-responsive frameshifting element.

In some embodiments, interaction between a polyamine and a polyamine-responsive frameshifting element results in a −1 ribosomal frameshift, and a reporter protein coding sequence is in the −1-frame of the polyamine-responsive frameshifting element.

### Reporter Proteins

In some embodiments, a reporter protein comprises a detectable protein.

In some embodiments, a reporter protein (e.g., a detectable protein) comprises a fluorescent protein. Non-limiting examples of fluorescent proteins include blue fluorescent proteins (e.g., EBFP2 and mTagBFP2), cyan fluorescent proteins (e.g., mTurquoise2, Cerulean3, mCFP, AmCyan1, and Midoriishi Cyan), green fluorescent proteins (e.g., AcGFP1, TurboGFP, Azami Green, ZsGreen, TagGFP2, Emerald, superfolder GFP, Clover, and mNeonGreen), yellow fluorescent proteins (e.g., mYFP, mVenus, mCitrine, TurboYFP, ZsYellow1, mPapaya1, and mBanana), orange fluorescent proteins (e.g., Kusabira Orange 2 (mK02), mOrange 2, TagRFP, and TagRFP-T), red fluorescent proteins (e.g., TurboRFP, tdTomato, DsRed-Express2, DsRed-Monomer, DsRed2, mStrawberry, AsRed2, mRuby2, FusionRed, and mCherry), far-red fluorescent proteins (e.g., HcRed1, mRaspberry, mKate2, E2-Crimson, mPlum, eqFP650, mNeptune2, and mCardinal), large stokes shift fluorescent proteins (e.g., T-Sapphire, mAmetrine, LSSmOrange, LSSmKate2, and mKeima), and near infra-red fluorescent proteins (e.g., iRFP670, iRFP682, iRFP702, iRFP713 (aka iRFP), and iRFP720), and variants thereof. For additional information on fluorescent proteins, see, e.g., Day & Davidson, The fluorescent protein palette: tools for cellular imaging, Chem Soc Rev. 38(10):2887-921 (2009), the contents of which are incorporated by reference herein in their entirety.

In some embodiments, a reporter protein comprises a yellow fluorescent protein (YFP) or a near-infrared fluorescent protein.

In some embodiments, a reporter protein comprises a YFP. In some embodiments, a YFP is an enhanced yellow fluorescent protein (eYFP). Non-limiting examples of yellow fluorescent proteins include mYFP, mVenus, mCitrine, TurboYFP, ZsYellow1, mPapaya1, and mBanana.

In some embodiments, a reporter protein comprises a near-infrared fluorescent protein. Non-limiting examples of near-infrared fluorescent proteins include iRFP670, iRFP682, iRFP702, iRFP713 (aka iRFP), iRFP720, miRFP670-2, miRF680, and miRF682. In some embodiments, a near-infrared fluorescent protein is miRFP670-2.

In some embodiments, a reporter protein (e.g., a detectable protein) comprises a non-fluorescent protein. In some embodiments, a non-fluorescent protein comprises a luciferase (e.g., encoded by bacterial lux, firefly luc, or Renilla Rluc). In some embodiments, a reporter protein comprises a toxin (e.g., diphtheria toxin (DT)). In some embodiments, a reporter protein comprises an enzyme (e.g., chloramphenicol acetyltransferase (CAT), or β-galactosidase (e.g., encoded by the LacZ gene)).

In some embodiments, a reporter protein has a half-life of less than 10 hours, for example, less than: 9, 8, 7, 6, 5, 4, 3, or 2 hours, or less than: 60, 50, 45, 40, 35, 30, 25, 20, or 15 minutes. In some embodiments, a reporter protein has a half-life of less than 2 hours.

In some embodiments, a reporter protein coding sequence encodes a destabilized fluorescent protein.

In some embodiments, a reporter protein coding sequence is fused to a sequence that induces reporter protein instability (e.g., induces reporter protein degradation). A non-limiting example paradigm for including protein degradation includes hydrophobic tagging (HyT) by appending a hydrophobic moiety (e.g., adamantane or Boc3-Arg). For additional information on induced protein degradation, see, e.g., Lai & Crews, Induced protein degradation: an emerging drug discovery paradigm, Nat Rev Drug Discov. 16(2):101-14 (2017), the contents of which are incorporated by reference herein in their entirety.

In some embodiments, a reporter protein coding sequence is fused to a protein-degradation tag coding sequence, a nuclear localization signal, or both.

In some embodiments, a reporter protein coding sequence is fused to a protein-degradation tag coding sequence. Non-limiting examples of protein-degradation tags include dihydrofolate reductase (DHFR) tag, auxin-inducible degradation (AID) tag, AID2 tag, IKAROS family zinc finger 3d (IKZF3d) tag, degradation TAG (dTAG), HaloTag, SMASh, and ubiquitin based tags. In some embodiments, a reporter protein coding sequence is fused to a DHFR tag.

In some embodiments, a reporter protein coding sequence is fused to a nuclear localization signal.

In some embodiments, a reporter protein coding sequence is fused to a polyamine-responsive element with a linker, for example, a glycine-serine sequence such as GGCTCCGGAGGT (SEQ ID NO:7).

### Control Proteins and Coding Sequences

In some embodiments, a polynucleotide further comprises a control protein coding sequence encoding a control protein.

In some embodiments, a reporter protein coding sequence and a control protein coding sequence are out of frame with each other. In some embodiments, a reporter protein coding sequence is in the +1-frame of the control protein coding sequence. In some embodiments, a reporter protein coding sequence is in the −1-frame (i.e., +2-frame) of the control protein coding sequence.

In some embodiments, a control protein coding sequence encodes a fluorescent protein. In some embodiments, a control protein coding sequence encodes mCherry.

### Vectors

The disclosure also provides, among other things, a vector (e.g., a plasmid) comprising any one or more of the polynucleotides disclosed herein.

The term “expression vector” refers to a replicable nucleic acid from which one or more proteins can be expressed when the expression vector is transformed into a suitable expression host cell.

In some embodiments, a vector (e.g., expression vector) further comprises an expression control polynucleotide sequence operably linked to the polynucleotide, a polynucleotide sequence encoding a selectable marker, or both.

In some embodiments, an expression control polynucleotide sequence comprises a promoter sequence, an enhancer sequence, or both. The term “promoter” refers to a region of DNA to which RNA polymerase binds and initiates the transcription of a gene.

In some embodiments, an expression control polynucleotide sequence comprises a constitutive promoter sequence.

In some embodiments, an expression control polynucleotide sequence comprises an inducible promoter sequence. In some embodiments, an inducible promoter is a chemically inducible promoter. In some embodiments, a chemically inducible promoter is positively inducible (e.g., tetracycline on (Tet-on), inducible by tetracycline (Tet) and/or doxycycline (Dox)). In some embodiments, an inducible promoter comprises a Dox-inducible promoter. In some embodiments, an inducible promoter comprises a Tet-inducible promoter. In some embodiments, an inducible promoter comprises a lecdysone-inducible promoter. In some embodiments, a chemically inducible promoter is negatively inducible (e.g., tetracycline off (Tet-on), suppressible by Tet and/or Dox). In some embodiments, an inducible promoter comprises a Dox-suppressible promoter. In some embodiments, an inducible promoter comprises a Tet-suppressible promoter.

The term “operably linked” means that the nucleic acid is positioned in the recombinant polynucleotide, e.g., vector, in such a way that enables expression of the nucleic acid under control of the element (e.g., promoter) to which it is linked.

The term “selectable marker element” is an element that confers a trait suitable for artificial selection. Selectable marker elements can be negative or positive selection markers.

### Cells

The disclosure also provides, among other things, a cell comprising any one or more of the polynucleotides or vectors disclosed herein.

In some embodiments, a cell is in vitro or ex vivo. In some embodiments, a cell is in vivo.

A cell may reside in or be obtained (e.g., isolated) from a biological entity containing expressed genetic materials. The biological entity may be a plant, animal, or microorganism. In some embodiments, a cell resides in a biological entity or a tissue. In some embodiments, a cell or its progeny was obtained (e.g., isolated) from a biological entity in vivo. In some embodiments, a cell or its progeny is cultured in vitro.

In some embodiments, a cell comprises or is an animal cell, a fungal cell (such as a yeast), or a plant cell.

In some embodiments, a cell comprises or is an animal cell. In some embodiments, a cell comprises or is a mammalian cell. In some embodiments, a mammalian cell is selected from a dog cell, a cat cell, a mouse cell, a rat cell, a hamster cell, a guinea pig cell, a horse cell, a pig cell, a sheep cell, a cow cell, a chimpanzee cell, a macaque cell, a cynomolgus monkey cell, or a human cell. In some embodiments, a cell is derived from a primate (e.g., a human or a non-human primate). In some embodiments, a cell comprises or is a human cell.

In some embodiments, a cell has or may have abnormal (e.g., dysregulated) polyamine metabolism (e.g., transport (e.g., uptake), synthesis, degradation, and/or modulation).

In some embodiments, a cell comprises a cancer cell. Non-limiting examples of cancers include leukemia (e.g., myelogenous leukemia) and solid tumors (e.g., sarcoma (e.g., osteosarcoma), pancreatic cancer, and neuroblastoma). For additional information on cancers or cancer cells with abnormal polyamine metabolism, see, e.g., Aouida (2010), Casero (2018), Gitto (2018), Ghandi (2019), Holbert (2022), Sekhar (2022), and Burns (2023), the contents of which are incorporated by reference herein in their entirety.

In some embodiments, a cell comprises or is a plant cell.

In some embodiments, a polynucleotide is integrated into the genomic DNA of a cell. In some embodiments, a polynucleotide is extrachromosomal.

In some embodiments, a cell is a single cell.

### Kits

Also provided herein, among other things, is a kit comprising a container and, optionally, an instruction for use, wherein the container comprises any one or more of the polynucleotides, vectors, or cells disclosed herein, or any combination of the foregoing.

### Methods

Also provided herein, among other things, is a method of reporting an intracellular polyamine (e.g., intracellular polyamine level and/or qualitative or quantitative change thereof), comprising providing any one or more of the cells disclosed herein, and obtaining a measurement comprising:
a) an expression of a reporter protein in the cell, or a temporal change thereof,
b) a ratio of expression between the reporter protein and a control protein, or a temporal change thereof, or
both a) and b).

In some embodiments, a method provides a quantitative readout of a level of an intracellular polyamine.

In some embodiments, obtaining a measurement uses fluorescent microscopy, flow cytometry, or both.

In some embodiments, a method further comprises calibrating a measurement against a second measurement.

In some embodiments, a second measurement quantifies intracellular polyamine using liquid chromatography-mass spectrometry (LC-MS).

In some embodiments, a method reports intracellular spermidine, spermine, or both. In some embodiments, a method reports intracellular spermidine. In some embodiments, a method reports intracellular spermine.

In some embodiments, a method reports:
a) a steady state level of an intracellular polyamine (e.g., spermidine),
b) a depletion of an intracellular polyamine (e.g., spermidine), an associated kinetic information, or both,
c) an import of an intracellular polyamine (e.g., spermidine), an associated kinetic information, or both,
or any combination of the foregoing.

In some embodiments, a reporter protein coding sequence is fused to a DHFR tag, and a method further comprises adding trimethoprim (TMP) to stabilize the reporter protein.

In some embodiments, a method is used for:
a) a high-throughput screen,
b) identifying a modulator of polyamine (e.g., spermidine) homeostasis,
c) performing pharmacokinetic characterization of a polyamine (e.g., spermidine)-targeting therapy, or
any combination of the foregoing.

In some embodiments, a high-throughput screen is a genome-wide CRISPR-Cas9 knockout screen.

Also provided herein, among other things, is a method of detecting and/or reporting an intracellular polyamine analog, comprising providing any one or more of the cells disclosed herein, and obtaining a measurement comprising:
a) an expression of a reporter protein in the cell, or a temporal change thereof,
b) a ratio of expression between the reporter protein and a control protein, or a temporal change thereof, or
both a) and b).

Non-limiting examples of polyamine analogs include Verlindamycin, N1,N11-Diethylnorspermine, α-methylspermidine (α-MeSpd), and N1,N12-dimethylspermine (Me2Spm).

### Sequences

Homo sapiens Ornithine Decarboxylase Antizyme 1 (OAZ1), Transcript Variant 1,

mRNA (NM_004152.3)
(SEQ ID NO: 1)
AGCATCTATAAAGGCGGGCGGCGGCAGAGGCGCCATTTTGCGAACGGCGAGCAGCGGCGGCGGCGCGGAGAGACGCA
GCGGAGGTTTTCCTGGTTTCGGACCCCAGCGGCCGGATGGTGAAATCCTCCCTGCAGCGGATCCTCAATAGCCACTG
CTTCGCCAGAGAGAAGGAAGGGGATAAACCCAGCGCCACCATCCACGCCAGCCGCACCATGCCGCTCCTAAGCCTGC
ACAGCCGCGGCGGCAGCAGCAGTGAGAGTTCCAGGGTCTCCCTCCACTGCTGTAGTAACCCGGGTCCGGGGCCTCGG
TGGTGCTCCTGATGCCCCTCACCCACCCCTGAAGATCCCAGGTGGGCGAGGGAATAGTCAGAGGGATCACAATCTTT
CAGCTAACTTATTCTACTCCGATGATCGGCTGAATGTAACAGAGGAACTAACGTCCAACGACAAGACGAGGATTCTC
AACGTCCAGTCCAGGCTCACAGACGCCAAACGCATTAACTGGCGAACAGTGCTGAGTGGCGGCAGCCTCTACATCGA
GATCCCGGGCGGCGCGCTGCCCGAGGGGAGCAAGGACAGCTTTGCAGTTCTCCTGGAGTTCGCTGAGGAGCAGCTGC
GAGCCGACCATGTCTTCATTTGCTTCCACAAGAACCGCGAGGACAGAGCCGCCTTGCTCCGAACCTTCAGCTTTTTG
GGCTTTGAGATTGTGAGACCGGGGCATCCCCTTGTCCCCAAGAGACCCGACGCTTGCTTCATGGCCTACACGTTCGA
GAGAGAGTCTTCGGGAGAGGAGGAGGAGTAGGGCCGCCTCGGGGCTGGGCATCCGGCCCCTGGGGCCACCCCTTGTC
AGCCGGGTGGGTAGGAACCGTAGACTCGCTCATCTCGCCTGGGTTTGTCCGCATGTTGTAATCGTGCAAATAAACGC
TCACTCCGAATTAGCGGTGTATTTCTTGAAGTTTAATATTGTGTTTGTGATACTGAAGTATTTGCTTTAATTCTAAA
TAAAAATTTATATTTTACTTTTTTATTGCTGGTTTAAGATGATTCAGATTATCCTTGTACTTTGAGGAGAAGTTTCT
TATTTGGAGTCTTTTGGAAACAGTCTTAGTCTTTTAACTTGGAAAGATGAGGTATTAATCCCCTCCATTGCTCTCCA
AAAGCCAATAAAGTGATTACACCCGAAAAAAAAAAAAAAA
OAZ1 (205-267)
(SEQ ID NO: 2)
TGCCCCTCACCCACCCCTGAAGATCCCAGGTGGGCGAGGGAATAGTCAGAGGGATCACAA
OAZ1 (202-267)
(SEQ ID NO: 3)
TCCTGATGCCCCTCACCCACCCCTGAAGATCCCAGGTGGGCGAGGGAATAGTCAGAGGGATCACAA
OAZ1 (185-275)
(SEQ ID NO: 4)
CGGGGCCTCGGTGGTGCTCCTGATGCCCCTCACCCACCCCTGAAGATCCCAGGTGGGCGAGGGAATAGTCAGAGGGA
TCACAATCTTTCAG
OAZ1 (106-275)
(SEQ ID NO: 5)
CTCCTAAGCCTGCACAGCCGCGGCGGCAGCAGCAGTGAGAGTTCCAGGGTCTCCCTCCACTGCTGTAGTAACCCGGG
TCCGGGGCCTCGGTGGTGCTCCTGATGCCCCTCACCCACCCCTGAAGATCCCAGGTGGGCGAGGGAATAGTCAGAGG
GATCACAATCTTTCAG
OAZ1 (1-275)
(SEQ ID NO: 6)
ATGGTGAAATCCTCCCTGCAGCGGATCCTCAATAGCCACTGCTTCGCCAGAGAGAAGGAAGGGGATAAACCCAGCGC
CACCATCCACGCCAGCCGCACCATGCCGCTCCTAAGCCTGCACAGCCGCGGCGGCAGCAGCAGTGAGAGTTCCAGGG
TCTCCCTCCACTGCTGTAGTAACCCGGGTCCGGGGCCTCGGTGGTGCTCCTGATGCCCCTCACCCACCCCTGAAGAT
CCCAGGTGGGCGAGGGAATAGTCAGAGGGATCACAATCTTTCAG

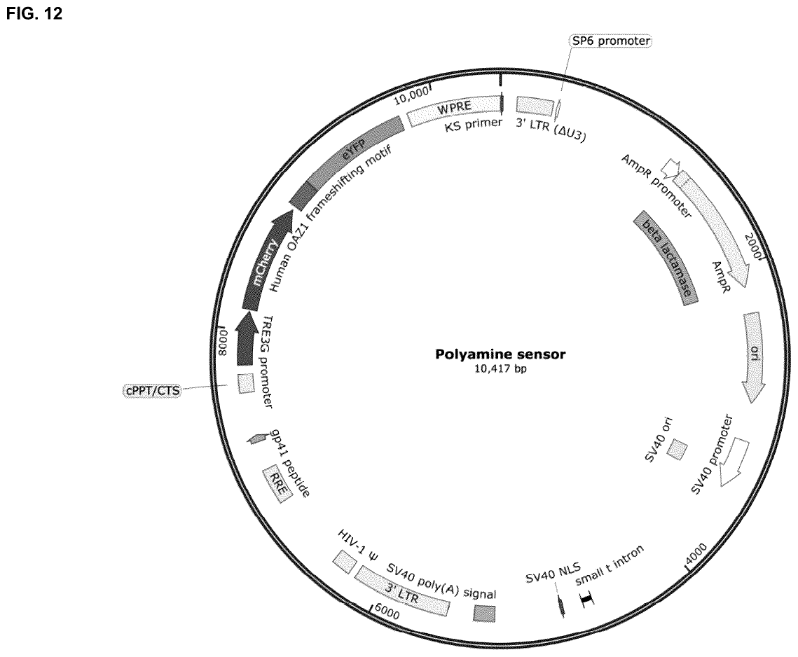

### A Non-Limiting Example of Reporter Construct (Also See FIG. 12)

(SEQ ID NO: 15)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTAAATAATAAACTAGTCTCCTAAGCCTGCACAGCCGCGGCGGCAGCAGCAGTGAGAGTTCCAGGGTCTCCC
TCCACTGCTGTAGTAACCCGGGTCCGGGGCCTCGGTGGTGCTCCTGATGCCCCTCACCCACCCCTGAAGATCCCAGG
TGGGCGAGGGAATAGTCAGAGGGATCACAATCTTTCAGgatccGTGAGCAAGGGCGAGGAGCTGTTCACCGGGGTGG
TGCCCATCCTGGTCGAGCTGGACGGCGACGTAAACGGCCACAAGTTCAGCGTGTCCGGCGAGGGCGAGGGCGATGCC
ACCTACGGCAAGCTGACCCTGAAGTTCATCTGCACCACCGGCAAGCTGCCCGTGCCCTGGCCCACCCTCGTGACCAC
CTTCGGCTACGGCCTGCAGTGCTTCGCCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGACGGCAACATCCTGGGGCACAA
GCTGGAGTACAACTACAACAGCCACAACGTCTATATCATGGCCGACAAGCAGAAGAACGGCATCAAGGTGAACTTCA
AGATCCGCCACAACATCGAGGACGGCAGCGTGCAGCTCGCCGACCACTACCAGCAGAACACCCCCATCGGCGACGGC
CCCGTGCTGCTGCCCGACAACCACTACCTGAGCTACCAGTCCAAACTGAGCAAAGACCCCAACGAGAAGCGCGATCA
CATGGTCCTGCTGGAGTTCGTGACCGCCGCCGGGATCACTCTCGGCATGGACGAGCTGTACAAGTGATGAATTgcGG
CCGCCTGACGTAACTAGTgTAAggcatgcaagcttgatatcaagcttatcgataatcaacctctggattacaaaatt
tgtgaaagattgactggtattcttaactatgttgctccttttacgctatgtggatacgctgctttaatgcctttgta
tcatgctattgcttcccgtatggctttcattttctcctccttgtataaatcctggttgctgtctctttatgaggagt
tgtggcccgttgtcaggcaacgtggcgtggtgtgcactgtgtttgctgacgcaacccccactggttggggcattgcc
accacctgtcagctcctttccgggactttcgctttccccctccctattgccacggcggaactcatcgccgcctgcct
tgcccgctgctggacaggggctcggctgttgggcactgacaattccgtggtgttgtcggggaaatcatcgtcctttc
cttggctgctcgcctgtgttgccacctggattctgcgcgggacgtccttctgctacgtcccttcggccctcaatcca
gcggaccttccttcccgcggcctgctgccggctctgcggcctcttccgcgtcttcgccttcgccctcagacgagtcg
gatctccctttgggccgcctccccgcat

pHR-Tre3G-mCherry-OAZ1 FS-eYFP (a Non-Limiting Example of Reporter Construct, Also See FIG. 12)

(SEQ ID NO: 16)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccccccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTAACACTAGTCTCCTAAGCCTGCACAGCCGCGGCGGCAGCAGCAGTGAGAGTTCCAGGGTCTCCCTCCACT
GCTGTAGTAACCCGGGTCCGGGGCCTCGGTGGTGCTCCTGATGCCCCTCACCCACCCCTGAAGATCCCAGGTGGGCG
AGGGAATAGTCAGAGGGATCACAATCTTTCAGgatccGTGAGCAAGGGCGAGGAGCTGTTCACCGGGGTGGTGCCCA
TCCTGGTCGAGCTGGACGGCGACGTAAACGGCCACAAGTTCAGCGTGTCCGGCGAGGGCGAGGGCGATGCCACCTAC
GGCAAGCTGACCCTGAAGTTCATCTGCACCACCGGCAAGCTGCCCGTGCCCTGGCCCACCCTCGTGACCACCTTCGG
CTACGGCCTGCAGTGCTTCGCCCGCTACCCCGACCACATGAAGCAGCACGACTTCTTCAAGTCCGCCATGCCCGAAG
GCTACGTCCAGGAGCGCACCATCTTCTTCAAGGACGACGGCAACTACAAGACCCGCGCCGAGGTGAAGTTCGAGGGC
GACACCCTGGTGAACCGCATCGAGCTGAAGGGCATCGACTTCAAGGAGGACGGCAACATCCTGGGGCACAAGCTGGA
GTACAACTACAACAGCCACAACGTCTATATCATGGCCGACAAGCAGAAGAACGGCATCAAGGTGAACTTCAAGATCC
GCCACAACATCGAGGACGGCAGCGTGCAGCTCGCCGACCACTACCAGCAGAACACCCCCATCGGCGACGGCCCCGTG
CTGCTGCCCGACAACCACTACCTGAGCTACCAGTCCAAACTGAGCAAAGACCCCAACGAGAAGCGCGATCACATGGT
CCTGCTGGAGTTCGTGACCGCCGCCGGGATCACTCTCGGCATGGACGAGCTGTACAAGTGATGAATTgcGGCCGCCT
GACGTAACTAGTgTAAggcatgcaagcttgatatcaagcttatcgataatcaacctctggattacaaaatttgtgaa
agattgactggtattcttaactatgttgctccttttacgctatgtggatacgctgctttaatgcctttgtatcatgc
tattgcttcccgtatggctttcattttctcctccttgtataaatcctggttgctgtctctttatgaggagttgtggc
ccgttgtcaggcaacgtggcgtggtgtgcactgtgtttgctgacgcaacccccactggttggggcattgccaccacc
tgtcagctcctttccgggactttcgctttccccctccctattgccacggcggaactcatcgccgcctgccttgcccg
ctgctggacaggggctcggctgttgggcactgacaattccgtggtgttgtcggggaaatcatcgtcctttccttggc
tgctcgcctgtgttgccacctggattctgcgcgggacgtccttctgctacgtcccttcggccctcaatccagcggac
cttccttcccgcggcctgctgccggctctgcggcctcttccgcgtcttcgccttcgccctcagacgagtcggatctc
cctttgggccgcctccccgcat

ODC1 KD (pLenti hU6-ODC1_sgRNA hUbC-dCas9-KRAB-T2A-Puro, See FIGS. 1F-1G)

(SEQ ID NO: 17)
cgccataactgccgcccccgcaacagacgacaaaccgagttctccagtcagtgacaaacttcacgtcagggtcccca
gatggtgccccagcccatctcacccgaataagagctttcccgcattagcgaaggcctcaagaccttgggttcttgcc
gcccaccatgccccccaccttgtttcaacgacctcacagcccgcctcacaagcgtcttccattcaagactcgggaac
agccgccattttgctgcgctccccccaacccccagttcagggcaaccttgctcgcggacccagactacagcccttgg
cggtctctccacacgcttccgtcccaccgagcggcccggcggccacgaaagccccggccagcccagcagcccgctac
tcaccaagtgacgatcacagcgatccacaaacaagaaccgcgacccaaatcccggctgcgacggaactagctgtgcc
acacccggcgcgtccttatataatcatcggcgttcaccgccccacggagatccctccgcagaatcgccgagaaggga
ctacttttcctcgcctgttccgctctctggaaagaaaaccagtgccctagagtcacccaagtcccgtcctaaaatgt
ccttctgctgatactggggttctaaggccgagtcttatgagcagcgggccgctgtcctgagcgtccgggcggaagga
tcaggacgctcgctgcgcccttcgtctgacgtggcagcgctcgccgtgaggaggggggcgcccgcgggaggcgccaa
aacccggcgcggaggccagatctggagccgacacgggttaattaattaagagggcctatttcccatgattccttcat
atttgcatatacgatacaaggctgttagagagataattggaattaatttgactgtaaacacaaagatattagtacaa
aatacgtgacgtagaaagtaataatttcttgggtagtttgcagttttaaaattatgttttaaaatggactatcatat
gcttaccgtaacttgaaagtatttcgatttcttggctttatatatcttgtggaaaggacgaaaCACCGggcggcggc
ggcTACAGGAgttttagagctagaaatagcaagttaaaataaggctagtccgttatcaacttgaaaaagtggcaccg
agtcggtgctttttttttaataacttcgtataatgtatgctatacgaagttattaattaaccaaactggatctctgc
tgtccctgtaataaacccgaaaattttgaatttttgtaatttgtttttgtaattctttagtttgtatgtctgttgct
attatgtctactattctttcccctgcactgtaccccccaatccccccttttcttttaaaattgtggatgaatactgc
catttgtctgcagaattggcgcacgcagtgccgatccgttcactaatcgaatggatctgtctctgtctctctctcca
ccttcttcttctattccttcgggcctgtcgggtcccctcggggttgggaggtgggtctgaaacgataatggtgaata
tccctgcctaactctattcactatagaaagtacagcaaaaactattcttaaacctaccaagcctcctactatcatta
tgaataattttatataccacagccaatttgttatgttaaaccaattccacaaacttgcccatttatctaattccaat
aattcttgttcattcttttcttgctggttttgcgattcttcaattaaggagtgtattaagcttgtgtaattgttaat
ttctctgtcccactccatccaggtcgtgtgattccaaatctgttccagagatttattactccaactagcattccaag
gcacagcagtggtgcaaatgagttttccagagcaaccccaaatccccaggagctgttgatcctttaggtatctttcc
acagccaggattcttgcctggagctgcttgatgccccagactgtgagttgcaacagatgctgttgcgcctcaatagc
cctcagcaaattgttctgctgctgcactataccagacaataattgtctggcctgtaccgtcagcgtcattgacgctg
cgcccatagtgcttcctgctgctcccaagaacccaaggaacaaagctcctattcccactgctcttttttctctctgc
accactcttctctttgccttggtgggtgctactcctaatggttcaatttttactactttatatttatataattcact
tctccaattgtccctcatatctcctcctccaggtctgaagatcagcggccgcttgctgtgcggtggtcttacttttg
ttttgctcttcctctatcttgtctaaagcttccttggtgtcttttatctctatcctttgatgcacacaatagagggt
tgctactgtattatataatgatctaagttcttctgatcctgtctgaagggatggttgtagctgtcccagtatttgtc
tacagccttctgatgtttctaacaggccaggattaactgcgaatcgttctagctccctgcttgcccatactatatgt
tttaatttatattttttctttccccctggccttaaccgaattttttcccatcgcgatctaattctcccccgcttaat
actgacgctctcgcacccatctctctccttctagcctccgctagtcaaaatttttggcgtactcaccagtcgccgcc
cctcgcctcttgccgtgcgcgcttcagcaagccgagtcctgcgtcgagagagctcctctggtttccctttcgctttc
aagtccctgttcgggcgccactgctagagattttccacactgactaaaagggtctgagggatctctagttaccagag
tcacacaacagacgggcacacactacttgaagcactcaaggcaagctttattgaggcttaagcagtgggttccctag
ttagccagagagctcccaggctcagatctggtctaaccagagagacccagtacaggcaaaacgcgctgcttatatag
acctcccaccgtacacgcctaccgcccatttgcgtcaatggggcggagttgttacgacattttggaaagtcccgttg
attttggtgccaaaacaaactcccattgacgtcaatggggtggagacttggaaatccccgtgagtcaaaccgctatc
cacgcccattgatgtactgccaaaaccgcatcaccatggtaatagcgatgactaatacgtagatgtactgccaagta
ggaaagtcccataaggtcatgtactgggcataatgccaggcgggccatttaccgtcattgacgtcaatagggggcgt
acttggcatatgatacacttgatgtactgccaagtgggcagtttaccgtaaatactccacccattgacgtcaatgga
aagtccctattggcgttactatgggaacatacgtcattattgacgtcaatgggcgggggtcgttgggcggtcagcca
ggcgggccatttaccgtaagttatgtaacgcggaactccatatatgggctatgaactaatgaccccgtaattgatta
ctattaataactagtcaataatcaatgtcaacgcgtatatctggcccgtacatcgcgaagcagcgcaaaacgcctaa
ccctaagcagattcttcatgcaattgtcggtcaagccttgccttgttgtagcttaaattttgctcgcgcactactca
gcgacctccaacacacaagcagggagcagatactggcttaactatgcggcatcagagcagattgtactgagagtgca
ccataggggatcgggagatctcccgatccgtcgacgtcaggtggcacttttcggggaaatgtgcgcggaacccctat
ttgtttatttttctaaatacattcaaatatgtatccgctcatgagacaataaccctgataaatgcttcaataatatt
gaaaaaggaagagtatgagtattcaacatttccgtgtcgcccttattcccttttttgcggcattttgccttcctgtt
tttgctcacccagaaacgctggtgaaagtaaaagatgctgaagatcagttgggtgcacgagtgggttacatcgaact
ggatctcaacagcggtaagatccttgagagttttcgccccgaagaacgttttccaatgatgagcacttttaaagttc
tgctatgtggcgcggtattatcccgtattgacgccgggcaagagcaactcggtcgccgcatacactattctcagaat
gacttggttgagtactcaccagtcacagaaaagcatcttacggatggcatgacagtaagagaattatgcagtgctgc
cataaccatgagtgataacactgcggccaacttacttctgacaacgatcggaggaccgaaggagctaaccgcttttt
tgcacaacatgggggatcatgtaactcgccttgatcgttgggaaccggagctgaatgaagccataccaaacgacgag
cgtgacaccacgatgcctgtagcaatggcaacaacgttgcgcaaactattaactggcgaactacttactctagcttc
ccggcaacaattaatagactggatggaggcggataaagttgcaggaccacttctgcgctcggcccttccggctggct
ggtttattgctgataaatctggagccggtgagcgtgggtctcgcggtatcattgcagcactggggccagatggtaag
ccctcccgtatcgtagttatctacacgacggggagtcaggcaactatggatgaacgaaatagacagatcgctgagat
aggtgcctcactgattaagcattggtaactgtcagaccaagtttactcatatatactttagattgatttaaaacttc
atttttaatttaaaaggatctaggtgaagatcctttttgataatctcatgaccaaaatcccttaacgtgagttttcg
ttccactgagcgtcagaccccgtagaaaagatcaaaggatcttcttgagatcctttttttctgcgcgtaatctgctg
cttgcaaacaaaaaaaccaccgctaccagcggtggtttgtttgccggatcaagagctaccaactctttttccgaagg
taactggcttcagcagagcgcagataccaaatactgttcttctagtgtagccgtagttaggccaccacttcaagaac
tctgtagcaccgcctacatacctcgctctgctaatcctgttaccagtggctgctgccagtggcgataagtcgtgtct
taccgggttggactcaagacgatagttaccggataaggcgcagcggtcgggctgaacggggggttcgtgcacacagc
ccagcttggagcgaacgacctacaccgaactgagatacctacagcgtgagctatgagaaagcgccacgcttcccgaa
gggagaaaggcggacaggtatccggtaagcggcagggtcggaacaggagagcgcacgagggagcttccagggggaaa
cgcctggtatctttatagtcctgtcgggtttcgccacctctgacttgagcgtcgatttttgtgatgctcgtcagggg
ggcggagcctatggaaaaacgccagcaacgcggcctttttacggttcctggccttttgctggccttttgctcacatg
ttctttcctgcgttatcccctgattctgtggataaccgtattaccgcctttgagtgagctgataccgctcgccgcag
ccgaacgaccgagcgcagcgagtcagtgagcgaggaagcggaagagcgcccaatacgcaaaccgcctctccccgcgc
gttggccgattcattaatgcagctggcacgacaggtttcccgactggaaagcgggcagtgagcgcaacgcaattaat
gtgagttagctcactcattaggcaccccaggctttacactttatgcttccggctcgtatgttgtgtggaattgtgag
cggataacaatttcacacaggaaacagctatgaccatgattacgccaagctctagctagaggtcgacggtatacaga
catgataagatacattgatgagtttggacaaaccacaactagaatgcagtgaaaaaaatgctttatttgtgaaattt
gtgatgctattgctttatttgtaaccattataagctgcaataaacaagttggggtgggcgaagaactccagcatgag
atccccgcgctggaggatcatccagccggcgtcccggaaaacgattccgaagcccaacctttcatagaaggcggcgg
tggaatcgaaatctcgtagcacgtgtcagtcctgctcctcggccacgaagtgcacgcagttgccggccgggtcgcgc
agggcgaactcccgcccccacggctgctcgccgatctcggtcatggccggcccggaggcgtcccggaagttcgtgga
cacgacctccgaccactcggcgtacagctcgtccaggccgcgcacccacacccaggccagggtgttgtccggcacca
cctggtcctggaccgcgctgatgaacagggtcacgtcgtcccggaccacaccggcgaagtcgtcctccacgaagtcc
cgggagaacccgagccggtcggtccagaactcgaccgctccggcgacgtcgcgcgcggtgagcaccggaacggcact
ggtcaacttggccatggtttagttcctcaccttgtcgtattatactatgccgatatactatgccgatgattaattgt
caacacgtgctgatcagatccgaaaatggatatacaagctcccgggagctttttgcaaaagcctaggcctccaaaaa
agcctcctcactacttctggaatagctcagaggcagaggcggcctcggcctctgcataaataaaaaaaattagtcag
ccatggggcggagaatgggcggaactgggcggagttaggggcgggatgggcggagttaggggcgggactatggttgc
tgactaattgagatgcatgctttgcatacttctgcctgctggggagcctggggactttccacacctggttgctgact
aattgagatgcatgctttgcatacttctgcctgctggggagcctggggactttccacaccctaactgacacacattc
cacagaattaattcgcgttaaatttttgttaaatcagctcattttttaaccaataggccgaaatcggcaaaatccct
tataaatcaaaagaatagaccgagatagggttgagtgttgttccagtttggaacaagagtccactattaaagaacgt
ggactccaacgtcaaagggcgaaaaaccgtctatcagggcgatggcccactacgtgaaccatcaccctaatcaagtt
ttttggggtcgaggtgccgtaaagcactaaatcggaaccctaaagggagcccccgatttagagcttgacggggaaag
ccggcgaacgtggcgagaaaggaagggaagaaagcgaaaggagcgggcgctagggcgctggcaagtgtagcggtcac
gctgcgcgtaaccaccacacccgccgcgcttaatgcgccgctacagggcgcgtggggataccccctagagccccagc
tggttctttccgcctcagaagccatagagcccaccgcatccccagcatgcctgctattgtcttcccaatcctccccc
ttgctgtcctgccccaccccaccccccagaatagaatgacacctactcagacaatgcgatgcaatttcctcatttta
ttaggaaaggacagtgggagtggcaccttccagggtcaaggaaggcacgggggaggggcaaacaacagatggctggc
aactagaaggcacagtcgaggctgatcagcgggtttaaacgggccctgctagagattttccacactgactaaaaggg
tctgagggatctctagttaccagagtcacacaacagacgggcacacactacttgaagcactcaaggcaagctttatt
gaggcttaagcagtgggttccctagttagccagagagctcccaggctcagatctggtctaaccagagagacccagta
cagtccggatgcagctctcgggccatgtgatgaaatgctaggcggctgtcaaacctccactctaatacttctctctc
cgggtcatccatcccatgcaggctcacagggtgtaacaagcgggtgttctctccttcattggcttcttctaccttct
cttgctcaactggtactagcttgtagcaccatccaaaggtcagtggatatctgatccctggccctggtgtgtagttc
tgccaatcagggaagtagccttgtgtgtggtagatccacagatcaaggatatcttgtcttcgttgggagtgaattag
cccttccagtccccccttttcttttaaaaagtggctaagatctacagctgccttgtaagtcattggtcttaaaggta
cctgaggtgtgactggaaaacccacctcctcctcctcttgtgcttctagccaggcacaatcagcattggtagctgct
gtattgctacttgtgattgctccatgtttttctaggtctcgaggtcgacggtatcgatgcggggaggcggcccaaag
ggagatccgactcgtctgagggcgaaggcgaagacgcggaagaggccgcagagccggcagcaggccgcgggaaggaa
ggtccgctggattgagggccgaagggacgtagcagaaggacgtcccgcgcagaatccaggtggcaacacaggcgagc
agccaaggaaaggacgatgatttccccgacaacaccacggaattgtcagtgcccaacagccgagcccctgtccagca
gcgggcaaggcaggcggcgatgagttccgccgtggcaatagggagggggaaagcgaaagtcccggaaaggagctgac
aggtggtggcaatgccccaaccagtgggggttgcgtcagcaaacacagtgcacaccacgccacgttgcctgacaacg
ggccacaactcctcataaagagacagcaaccaggatttatacaaggaggagaaaatgaaagccatacgggaagcaat
agcatgatacaaaggcattaaagcagcgtatccacatagcgtaaaaggagcaacatagttaagaataccagtcaatc
tttcacaaattttgtaatccagaggttgattatcgataagcttgatatcgaattcataacttcgtataatgtatgct
atacgaagttatctattatcaaccggtcgtgctgcagaagctagtaacggccgccagtgtgctggtcaggcaccggg
cttgcgggtcatgcaccaggtgcgcggtccttcgggcacctcgacgtcggcggtgacggtgaagccgagccgctcgt
agaaggggaggttgcggggcgcggaggtctccaggaaggcgggcaccccggcgcgctcggccgcctccactccgggg
agcacgacggcgctgcccagacccttgccctggtggtcgggcgacacgccgacggtggccaggaaccacgcgggctc
cttgggccggtgcggcgccaggaggccttccatctgttgctgcgcggccagccgggaaccgctcaactcggccatgc
gcgggccgatctcggcgaacaccgcccccgcttcgacgctctccggcgtggtccagaccgccaccgcggcgccgtcg
tccgcgacccacaccttgccgatgtcgagcccgacgcgcgtgaggaagagttcttgcagctcggtgacccgctcgat
gtggcggtccggatcgacggtgtggcgcgtggcggggtagtcggcgaacgcggcggcgagggtgcgtacggccctgg
ggacgtcgtcgcgggtggcgaggcgcaccgtgggcttgtactcggtcatagggccgggattctcctccacgtcaccg
catgttagaagacttcctctgccctcgctagcaactttgcgtttctttttcggaactgatgatttgatttcaaatgc
agtctctgaatcaggatgggtctcttggtgaatttctctctccaccagccagggctcttctcccttctccaaccgga
ggatcacatctggcttagtaagctgataacccaaggaaaccaggttcttatagttctccagcatcacatttctgtac
aggatctgctgagcagtgtccagcagcttccactcctccctggtgaagtccacaaacacatccttgaaggtcaccag
tgtccgggaccaggcagtcagtgacttagcatcgctagccaccttcctcttcttcttggggtcagccctgctgtctc
caccgagctgagagaggtcgattcttgtttcatagagccccgtaattgactgatgaatcagtgtggcgtccaggacc
tcctttgtagaggtgtaccgctttctgtctatggtggtgtcgaagtacttgaaggctgcaggcgcgcccaagttggt
cagagtaaacaagtggataatgttttctgcctgctccctgatgggcttatccctgtgcttattgtaagcagaaagca
ccttatcgaggttagcgtcggcgaggatcactcttttggagaattcgcttatttgctcgatgatctcatcaaggtag
tgtttgtgttgttccacgaacagctgcttctgctcattatcttcgggagaccctttgagcttttcatagtggctggc
cagatacaagaaattaacgtatttagagggcagtgccagctcgttacctttctgcagctcgcccgcactagcgagca
ttcgtttccggccgttttcaagctcaaagagagagtacttgggaagcttaatgatgaggtcttttttgacctcttta
tatcctttcgcctcgagaaagtcgatggggtttttttcgaagcttgatcgctccatgattgtgatgcccagcagttc
cttgacgcttttgagttttttagacttccctttctccactttggccacaaccagtacactgtaagcgactgtaggag
aatcgaatccgccgtatttcttggggtcccaatcttttttgcgtgcgatcagcttgtcgctgttccttttcgggagg
atactttccttggagaagcctccggtctgtacttcggtctttttaacgatgttcacctgcggcatggacaggacctt
ccggactgtcgcgaaatccctacccttgtcccacacgatttctcctgtttctccgtttgtttcgataagtggtcgct
tccgaatctctccattggccagtgtaatctcggtcttgaaaaaattcataatattgctgtaaaagaagtacttagcg
gtggccttgcctatttcctgctcagactttgcgatcattttcctaacatcgtacactttatagtctccgtaaacaaa
ttcagattcaagcttgggatattttttgataagtgcagtgcctaccactgcattcaggtaggcatcatgcgcatggt
ggtaattgttgatctctctcaccttataaaactgaaagtcctttctgaaatctgagaccagcttagacttcagagta
ataactttcacctctcgaatcagtttgtcattttcatcgtacttggtgttcatgcgtgaatcgagaatttgggccac
gtgcttggtgatctggcgtgtctcaacaagctgccttttgatgaagccggctttatccaactcagacaggccacctc
gttcagccttagtcagattatcgaacttccgttgtgtgatcagtttggcgttcagcagctgccgccaataatttttc
attttcttgacaacttcttctgaggggacgttatcactcttccctctatttttatcggatcttgtcaacactttatt
atcaatagaatcatctttgagaaaagactggggcacgatggcatccacgtcgtagtcggagagccgattgatgtcca
gttcctgatccacgtacatgtccctgccgttctgcaggtagtacaggtagagcttctcattctgaagctgggtgttt
tcaactgggtgttccttaaggatttgggaccccagttcttttataccctcttcaatcctcttcatcctttccctact
gttcttctgtcccttctgggtagtttggttctctcgggccatctcgataacgatattctcgggcttatgccttccca
ttactttgacgagttcatccacgaccttaacggtctgcagtattccctttttgatagctgggctacctgcaagatta
gcgatgtgctcgtgaagactgtccccctggccagaaacttgtgctttctggatgtcctccttaaaggtgagagagtc
atcatggatcaactgcatgaagttccggttggcaaatccatcggacttaagaaaatccaggattgtctttccactct
gcttgtctcggatcccattgatcagttttcttgacagccgcccccatcctgtatatcggcgcctcttgagctgtttc
atgactttgtcgtcgaagagatgagcgtaagttttcaagcgttcttcaatcatctccctatcttcaaacaacgtaag
ggtgaggacaatgtcctcaagaatgtcctcgttctcctcattgtccaggaagtccttgtctttaatgattttcagga
gatcgtgatacgttcccagggatgcgttgaagcgatcctccactccgctgatttcaacagagtcgaaacattcaatc
tttttgaaatagtcttctttgagctgtttcacggtaactttccggttcgtcttgaagaggaggtccacgatagcttt
cttctgctctccagacaggaatgctggctttctcatcccttctgtgacgtatttgaccttggtgagctcgttataaa
ctgtgaagtactcgtacagcagagagtgtttaggaagcaccttttcgttaggcagatttttatcaaagttagtcatc
ctttcgatgaaggactgggcagaggcccccttatccacgacttcctcgaagttccagggagtgatggtctcttctga
tttgcgagtcatccacgcgaatctggaatttccccgggcgagggggcctacatagtagggtatccgaaatgtgagga
ttttctcaatcttttccctgttatctttcaaaaaggggtagaaatcctcttgccgcctgaggatagcgtgcagttcg
cccaggtgaatctggtgggggatgcttccattgtcgaaagtgcgctgtttgcgcaacagatcttctctgttaagctt
taccagcagctcctcggtgccgtccattttttccaagatgggcttaataaatttgtaaaattcctcctggcttgctc
cgccgtcaatgtatccggcgtagccatttttagactgatcgaagaaaatttccttgtacttctcaggcagttgctgt
ctgacaagggccttcagcaaagtcaagtcttggtggtgctcatcatagcgcttgatcatactagcgctcagcggagc
tttggtgatctccgtgttcactcgcagaatatcactcagcagaatggcgtctgacaggttctttgccgccaaaaaaa
ggtctgcgtactggtcgccgatctgggccagcagattgtcgagatcatcatcgtaggtgtctttgctcagttgaagc
ttggcatcttcggccaggtcgaagttagatttaaagttgggggtcagcccgagtgacagggcgataagattaccaaa
caggccgttcttcttctccccagggagctgtgcgatgaggttttcgagccgccgggatttggacagcctagcgctca
ggattgctttggcgtcaactccggatgcgttgatcgggttctcttcgaaaagctgattgtaagtctgaaccagttgg
ataaagagtttgtcgacatcgctgttgtctgggttcaggtccccctcgatgaggaagtgtccccgaaatttgatcat
atgcgccagcgcgagatagatcaaccgcaagtcagccttatcagtactgtctacaagcttcttcctcagatgatata
tggttgggtacttttcatggtacgccacctcgtccacgatattgccaaagattgggtggcgctcgtgctttttatcc
tcctccaccaaaaaggactcctccagcctatggaagaaagagtcatccaccttagccatctcattactaaagatctc
ctgcaggtagcagatccgattctttctgcgggtatatctgcgccgtgctgttcttttgagccgcgtggcttcggcgg
tttccccggagtcgaacaggagggcgccaatgaggttcttctttatgctgtggcgatcggtattgcccagaactttg
aattttttgctcggcaccttgtactcgtccgtaatgacggcccagccgacgctgtttgtgccgatggcgagcccaat
ggagtacttcttgtccattccgcggcccaccttcctcttcttcttgggggccatcttgtcatcgtcatccttgtaat
cgatgtcatgatctttataatcaccgtcatggtctttgtagtccatggtggctctagagtcgacctgcagcccaagc
ttcgtctaacaaaaaagccaaaaacggccagaatttagcggacaatttactagtctaacactgaaaattacatattg
acccaaatgattacatttcaaaaggtgcctaaaaaacttcacaaaacacactcgccaaccccgagcgcatagttcaa
aaccggagcttcagctacttaagaagataggtacataaaaccgaccaaagaaactgacgcctcacttatccctcccc
tcaccagaggtccggcgcctgtcgattcaggagagcctaccctaggcccgaaccctgcgtcctgcgacggagaaaag
cctaccgcacacctaccggcaggtggccccaccctgcattataagccaacagaacgggtgacgtcacgacacgacga
gggcgcgcgctcccaaaggtacgggtgcactgcccaacggcac

SRM KD (pLenti hU6-SRM_sgRNA hUbC-dCas9-KRAB-T2A-Puro, See FIGS. 1F-1G)

(SEQ ID NO: 17)
gtgccgttgggcagtgcacccgtacctttgggagcgcgcgccctcgtcgtgtcgtgacgtcacccgttctgttggct
tataatgcagggtggggccacctgccggtaggtgtgcggtaggcttttctccgtcgcaggacgcagggttcgggcct
agggtaggctctcctgaatcgacaggcgccggacctctggtgaggggagggataagtgaggcgtcagtttctttggt
cggttttatgtacctatcttcttaagtagctgaagctccggttttgaactatgcgctcggggttggcgagtgtgttt
tgtgaagttttttaggcaccttttgaaatgtaatcatttgggtcaatatgtaattttcagtgttagactagtaaatt
gtccgctaaattctggccgtttttggcttttttgttagacgaagcttgggctgcaggtcgactctagagccaccatg
gactacaaagaccatgacggtgattataaagatcatgacatcgattacaaggatgacgatgacaagatggcccccaa
gaagaagaggaaggtgggccgcggaatggacaagaagtactccattgggctcgccatcggcacaaacagcgtcggct
gggccgtcattacggacgagtacaaggtgccgagcaaaaaattcaaagttctgggcaataccgatcgccacagcata
aagaagaacctcattggcgccctcctgttcgactccggggaaaccgccgaagccacgcggctcaaaagaacagcacg
gcgcagatatacccgcagaaagaatcggatctgctacctgcaggagatctttagtaatgagatggctaaggtggatg
actctttcttccataggctggaggagtcctttttggtggaggaggataaaaagcacgagcgccacccaatctttggc
aatatcgtggacgaggtggcgtaccatgaaaagtacccaaccatatatcatctgaggaagaagcttgtagacagtac
tgataaggctgacttgcggttgatctatctcgcgctggcgcatatgatcaaatttcggggacacttcctcatcgagg
gggacctgaacccagacaacagcgatgtcgacaaactctttatccaactggttcagacttacaatcagcttttcgaa
gagaacccgatcaacgcatccggagttgacgccaaagcaatcctgagcgctaggctgtccaaatcccggcggctcga
aaacctcatcgcacagctccctggggagaagaagaacggcctgtttggtaatcttatcgccctgtcactcgggctga
cccccaactttaaatctaacttcgacctggccgaagatgccaagcttcaactgagcaaagacacctacgatgatgat
ctcgacaatctgctggcccagatcggcgaccagtacgcagacctttttttggcggcaaagaacctgtcagacgccat
tctgctgagtgatattctgcgagtgaacacggagatcaccaaagctccgctgagcgctagtatgatcaagcgctatg
atgagcaccaccaagacttgactttgctgaaggcccttgtcagacagcaactgcctgagaagtacaaggaaattttc
ttcgatcagtctaaaaatggctacgccggatacattgacggcggagcaagccaggaggaattttacaaatttattaa
gcccatcttggaaaaaatggacggcaccgaggagctgctggtaaagcttaacagagaagatctgttgcgcaaacagc
gcactttcgacaatggaagcatcccccaccagattcacctgggcgaactgcacgctatcctcaggcggcaagaggat
ttctacccctttttgaaagataacagggaaaagattgagaaaatcctcacatttcggataccctactatgtaggccc
cctcgcccggggaaattccagattcgcgtggatgactcgcaaatcagaagagaccatcactccctggaacttcgagg
aagtcgtggataagggggcctctgcccagtccttcatcgaaaggatgactaactttgataaaaatctgcctaacgaa
aaggtgcttcctaaacactctctgctgtacgagtacttcacagtttataacgagctcaccaaggtcaaatacgtcac
agaagggatgagaaagccagcattcctgtctggagagcagaagaaagctatcgtggacctcctcttcaagacgaacc
ggaaagttaccgtgaaacagctcaaagaagactatttcaaaaagattgaatgtttcgactctgttgaaatcagcgga
gtggaggatcgcttcaacgcatccctgggaacgtatcacgatctcctgaaaatcattaaagacaaggacttcctgga
caatgaggagaacgaggacattcttgaggacattgtcctcacccttacgttgtttgaagatagggagatgattgaag
aacgcttgaaaacttacgctcatctcttcgacgacaaagtcatgaaacagctcaagaggcgccgatatacaggatgg
gggcggctgtcaagaaaactgatcaatgggatccgagacaagcagagtggaaagacaatcctggattttcttaagtc
cgatggatttgccaaccggaacttcatgcagttgatccatgatgactctctcacctttaaggaggacatccagaaag
cacaagtttctggccagggggacagtcttcacgagcacatcgctaatcttgcaggtagcccagctatcaaaaaggga
atactgcagaccgttaaggtcgtggatgaactcgtcaaagtaatgggaaggcataagcccgagaatatcgttatcga
gatggcccgagagaaccaaactacccagaagggacagaagaacagtagggaaaggatgaagaggattgaagagggta
taaaagaactggggtcccaaatccttaaggaacacccagttgaaaacacccagcttcagaatgagaagctctacctg
tactacctgcagaacggcagggacatgtacgtggatcaggaactggacatcaatcggctctccgactacgacgtgga
tgccatcgtgccccagtcttttctcaaagatgattctattgataataaagtgttgacaagatccgataaaaatagag
ggaagagtgataacgtcccctcagaagaagttgtcaagaaaatgaaaaattattggcggcagctgctgaacgccaaa
ctgatcacacaacggaagttcgataatctgactaaggctgaacgaggtggcctgtctgagttggataaagccggctt
catcaaaaggcagcttgttgagacacgccagatcaccaagcacgtggcccaaattctcgattcacgcatgaacacca
agtacgatgaaaatgacaaactgattcgagaggtgaaagttattactctgaagtctaagctggtctcagatttcaga
aaggactttcagttttataaggtgagagagatcaacaattaccaccatgcgcatgatgcctacctgaatgcagtggt
aggcactgcacttatcaaaaaatatcccaagcttgaatctgaatttgtttacggagactataaagtgtacgatgtta
ggaaaatgatcgcaaagtctgagcaggaaataggcaaggccaccgctaagtacttcttttacagcaatattatgaat
tttttcaagaccgagattacactggccaatggagagattcggaagcgaccacttatcgaaacaaacggagaaacagg
agaaatcgtgtgggacaagggtagggatttcgcgacagtccggaaggtcctgtccatgccgcaggtgaacatcgtta
aaaagaccgaagtacagaccggaggcttctccaaggaaagtatcctcccgaaaaggaacagcgacaagctgatcgca
cgcaaaaaagattgggaccccaagaaatacggcggattcgattctcctacagtcgcttacagtgtactggttgtggc
caaagtggagaaagggaagtctaaaaaactcaaaagcgtcaaggaactgctgggcatcacaatcatggagcgatcaa
gcttcgaaaaaaaccccatcgactttctcgaggcgaaaggatataaagaggtcaaaaaagacctcatcattaagctt
cccaagtactctctctttgagcttgaaaacggccggaaacgaatgctcgctagtgcgggcgagctgcagaaaggtaa
cgagctggcactgccctctaaatacgttaatttcttgtatctggccagccactatgaaaagctcaaagggtctcccg
aagataatgagcagaagcagctgttcgtggaacaacacaaacactaccttgatgagatcatcgagcaaataagcgaa
ttctccaaaagagtgatcctcgccgacgctaacctcgataaggtgctttctgcttacaataagcacagggataagcc
catcagggagcaggcagaaaacattatccacttgtttactctgaccaacttgggcgcgcctgcagccttcaagtact
tcgacaccaccatagacagaaagcggtacacctctacaaaggaggtcctggacgccacactgattcatcagtcaatt
acggggctctatgaaacaagaatcgacctctctcagctcggtggagacagcagggctgaccccaagaagaagaggaa
ggtggctagcgatgctaagtcactgactgcctggtcccggacactggtgaccttcaaggatgtgtttgtggacttca
ccagggaggagtggaagctgctggacactgctcagcagatcctgtacagaaatgtgatgctggagaactataagaac
ctggtttccttgggttatcagcttactaagccagatgtgatcctccggttggagaagggagaagagccctggctggt
ggagagagaaattcaccaagagacccatcctgattcagagactgcatttgaaatcaaatcatcagttccgaaaaaga
aacgcaaagttgctagcgagggcagaggaagtcttctaacatgcggtgacgtggaggagaatcccggccctatgacc
gagtacaagcccacggtgcgcctcgccacccgcgacgacgtccccagggccgtacgcaccctcgccgccgcgttcgc
cgactaccccgccacgcgccacaccgtcgatccggaccgccacatcgagcgggtcaccgagctgcaagaactcttcc
tcacgcgcgtcgggctcgacatcggcaaggtgtgggtcgcggacgacggcgccgcggtggcggtctggaccacgccg
gagagcgtcgaagcgggggcggtgttcgccgagatcggcccgcgcatggccgagttgagcggttcccggctggccgc
gcagcaacagatggaaggcctcctggcgccgcaccggcccaaggagcccgcgtggttcctggccaccgtcggcgtgt
cgcccgaccaccagggcaagggtctgggcagcgccgtcgtgctccccggagtggaggcggccgagcgcgccggggtg
cccgccttcctggagacctccgcgccccgcaacctccccttctacgagcggctcggcttcaccgtcaccgccgacgt
cgaggtgcccgaaggaccgcgcacctggtgcatgacccgcaagcccggtgcctgaccagcacactggcggccgttac
tagcttctgcagcacgaccggttgataatagataacttcgtatagcatacattatacgaagttatgaattcgatatc
aagcttatcgataatcaacctctggattacaaaatttgtgaaagattgactggtattcttaactatgttgctccttt
tacgctatgtggatacgctgctttaatgcctttgtatcatgctattgcttcccgtatggctttcattttctcctcct
tgtataaatcctggttgctgtctctttatgaggagttgtggcccgttgtcaggcaacgtggcgtggtgtgcactgtg
tttgctgacgcaacccccactggttggggcattgccaccacctgtcagctcctttccgggactttcgctttccccct
ccctattgccacggcggaactcatcgccgcctgccttgcccgctgctggacaggggctcggctgttgggcactgaca
attccgtggtgttgtcggggaaatcatcgtcctttccttggctgctcgcctgtgttgccacctggattctgcgcggg
acgtccttctgctacgtcccttcggccctcaatccagcggaccttccttcccgcggcctgctgccggctctgcggcc
tcttccgcgtcttcgccttcgccctcagacgagtcggatctccctttgggccgcctccccgcatcgataccgtcgac
ctcgagacctagaaaaacatggagcaatcacaagtagcaatacagcagctaccaatgctgattgtgcctggctagaa
gcacaagaggaggaggaggtgggttttccagtcacacctcaggtacctttaagaccaatgacttacaaggcagctgt
agatcttagccactttttaaaagaaaaggggggactggaagggctaattcactcccaacgaagacaagatatccttg
atctgtggatctaccacacacaaggctacttccctgattggcagaactacacaccagggccagggatcagatatcca
ctgacctttggatggtgctacaagctagtaccagttgagcaagagaaggtagaagaagccaatgaaggagagaacac
ccgcttgttacaccctgtgagcctgcatgggatggatgacccggagagagaagtattagagtggaggtttgacagcc
gcctagcatttcatcacatggcccgagagctgcatccggactgtactgggtctctctggttagaccagatctgagcc
tgggagctctctggctaactagggaacccactgcttaagcctcaataaagcttgccttgagtgcttcaagtagtgtg
tgcccgtctgttgtgtgactctggtaactagagatccctcagacccttttagtcagtgtggaaaatctctagcaggg
cccgtttaaacccgctgatcagcctcgactgtgccttctagttgccagccatctgttgtttgcccctcccccgtgcc
ttccttgaccctggaaggtgccactcccactgtcctttcctaataaaatgaggaaattgcatcgcattgtctgagta
ggtgtcattctattctggggggtggggtggggcaggacagcaagggggaggattgggaagacaatagcaggcatgct
ggggatgcggtgggctctatggcttctgaggcggaaagaaccagctggggctctagggggtatccccacgcgccctg
tagcggcgcattaagcgcggcgggtgtggtggttacgcgcagcgtgaccgctacacttgccagcgccctagcgcccg
ctcctttcgctttcttcccttcctttctcgccacgttcgccggctttccccgtcaagctctaaatcgggggctccct
ttagggttccgatttagtgctttacggcacctcgaccccaaaaaacttgattagggtgatggttcacgtagtgggcc
atcgccctgatagacggtttttcgccctttgacgttggagtccacgttctttaatagtggactcttgttccaaactg
gaacaacactcaaccctatctcggtctattcttttgatttataagggattttgccgatttcggcctattggttaaaa
aatgagctgatttaacaaaaatttaacgcgaattaattctgtggaatgtgtgtcagttagggtgtggaaagtcccca
ggctccccagcaggcagaagtatgcaaagcatgcatctcaattagtcagcaaccaggtgtggaaagtccccaggctc
cccagcaggcagaagtatgcaaagcatgcatctcaattagtcagcaaccatagtcccgcccctaactccgcccatcc
cgcccctaactccgcccagttccgcccattctccgccccatggctgactaattttttttatttatgcagaggccgag
gccgcctctgcctctgagctattccagaagtagtgaggaggcttttttggaggcctaggcttttgcaaaaagctccc
gggagcttgtatatccattttcggatctgatcagcacgtgttgacaattaatcatcggcatagtatatcggcatagt
ataatacgacaaggtgaggaactaaaccatggccaagttgaccagtgccgttccggtgctcaccgcgcgcgacgtcg
ccggagcggtcgagttctggaccgaccggctcgggttctcccgggacttcgtggaggacgacttcgccggtgtggtc
cgggacgacgtgaccctgttcatcagcgcggtccaggaccaggtggtgccggacaacaccctggcctgggtgtgggt
gcgcggcctggacgagctgtacgccgagtggtcggaggtcgtgtccacgaacttccgggacgcctccgggccggcca
tgaccgagatcggcgagcagccgtgggggcgggagttcgccctgcgcgacccggccggcaactgcgtgcacttcgtg
gccgaggagcaggactgacacgtgctacgagatttcgattccaccgccgccttctatgaaaggttgggcttcggaat
cgttttccgggacgccggctggatgatcctccagcgcggggatctcatgctggagttcttcgcccaccccaacttgt
ttattgcagcttataatggttacaaataaagcaatagcatcacaaatttcacaaataaagcatttttttcactgcat
tctagttgtggtttgtccaaactcatcaatgtatcttatcatgtctgtataccgtcgacctctagctagagcttggc
gtaatcatggtcatagctgtttcctgtgtgaaattgttatccgctcacaattccacacaacatacgagccggaagca
taaagtgtaaagcctggggtgcctaatgagtgagctaactcacattaattgcgttgcgctcactgcccgctttccag
tcgggaaacctgtcgtgccagctgcattaatgaatcggccaacgcgcggggagaggcggtttgcgtattgggcgctc
ttccgcttcctcgctcactgactcgctgcgctcggtcgttcggctgcggcgagcggtatcagctcactcaaaggcgg
taatacggttatccacagaatcaggggataacgcaggaaagaacatgtgagcaaaaggccagcaaaaggccaggaac
cgtaaaaaggccgcgttgctggcgtttttccataggctccgcccccctgacgagcatcacaaaaatcgacgctcaag
tcagaggtggcgaaacccgacaggactataaagataccaggcgtttccccctggaagctccctcgtgcgctctcctg
ttccgaccctgccgcttaccggatacctgtccgcctttctcccttcgggaagcgtggcgctttctcatagctcacgc
tgtaggtatctcagttcggtgtaggtcgttcgctccaagctgggctgtgtgcacgaaccccccgttcagcccgaccg
ctgcgccttatccggtaactatcgtcttgagtccaacccggtaagacacgacttatcgccactggcagcagccactg
gtaacaggattagcagagcgaggtatgtaggcggtgctacagagttcttgaagtggtggcctaactacggctacact
agaagaacagtatttggtatctgcgctctgctgaagccagttaccttcggaaaaagagttggtagctcttgatccgg
caaacaaaccaccgctggtagcggtggtttttttgtttgcaagcagcagattacgcgcagaaaaaaaggatctcaag
aagatcctttgatcttttctacggggtctgacgctcagtggaacgaaaactcacgttaagggattttggtcatgaga
ttatcaaaaaggatcttcacctagatccttttaaattaaaaatgaagttttaaatcaatctaaagtatatatgagta
aacttggtctgacagttaccaatgcttaatcagtgaggcacctatctcagcgatctgtctatttcgttcatccatag
ttgcctgactccccgtcgtgtagataactacgatacgggagggcttaccatctggccccagtgctgcaatgataccg
cgagacccacgctcaccggctccagatttatcagcaataaaccagccagccggaagggccgagcgcagaagtggtcc
tgcaactttatccgcctccatccagtctattaattgttgccgggaagctagagtaagtagttcgccagttaatagtt
tgcgcaacgttgttgccattgctacaggcatcgtggtgtcacgctcgtcgtttggtatggcttcattcagctccggt
tcccaacgatcaaggcgagttacatgatcccccatgttgtgcaaaaaagcggttagctccttcggtcctccgatcgt
tgtcagaagtaagttggccgcagtgttatcactcatggttatggcagcactgcataattctcttactgtcatgccat
ccgtaagatgcttttctgtgactggtgagtactcaaccaagtcattctgagaatagtgtatgcggcgaccgagttgc
tcttgcccggcgtcaatacgggataataccgcgccacatagcagaactttaaaagtgctcatcattggaaaacgttc
ttcggggcgaaaactctcaaggatcttaccgctgttgagatccagttcgatgtaacccactcgtgcacccaactgat
cttcagcatcttttactttcaccagcgtttctgggtgagcaaaaacaggaaggcaaaatgccgcaaaaaagggaata
agggcgacacggaaatgttgaatactcatactcttcctttttcaatattattgaagcatttatcagggttattgtct
catgagcggatacatatttgaatgtatttagaaaaataaacaaataggggttccgcgcacatttccccgaaaagtgc
cacctgacgtcgacggatcgggagatctcccgatcccctatggtgcactctcagtacaatctgctctgatgccgcat
agttaagccagtatctgctccctgcttgtgtgttggaggtcgctgagtagtgcgcgagcaaaatttaagctacaaca
aggcaaggcttgaccgacaattgcatgaagaatctgcttagggttaggcgttttgcgctgcttcgcgatgtacgggc
cagatatacgcgttgacattgattattgactagttattaatagtaatcaattacggggtcattagttcatagcccat
atatggagttccgcgttacataacttacggtaaatggcccgcctggctgaccgcccaacgacccccgcccattgacg
tcaataatgacgtatgttcccatagtaacgccaatagggactttccattgacgtcaatgggtggagtatttacggta
aactgcccacttggcagtacatcaagtgtatcatatgccaagtacgccccctattgacgtcaatgacggtaaatggc
ccgcctggcattatgcccagtacatgaccttatgggactttcctacttggcagtacatctacgtattagtcatcgct
attaccatggtgatgcggttttggcagtacatcaatgggcgtggatagcggtttgactcacggggatttccaagtct
ccaccccattgacgtcaatgggagtttgttttggcaccaaaatcaacgggactttccaaaatgtcgtaacaactccg
ccccattgacgcaaatgggcggtaggcgtgtacggtgggaggtctatataagcagcgcgttttgcctgtactgggtc
tctctggttagaccagatctgagcctgggagctctctggctaactagggaacccactgcttaagcctcaataaagct
tgccttgagtgcttcaagtagtgtgtgcccgtctgttgtgtgactctggtaactagagatccctcagacccttttag
tcagtgtggaaaatctctagcagtggcgcccgaacagggacttgaaagcgaaagggaaaccagaggagctctctcga
cgcaggactcggcttgctgaagcgcgcacggcaagaggcgaggggcggcgactggtgagtacgccaaaaattttgac
tagcggaggctagaaggagagagatgggtgcgagagcgtcagtattaagcgggggagaattagatcgcgatgggaaa
aaattcggttaaggccagggggaaagaaaaaatataaattaaaacatatagtatgggcaagcagggagctagaacga
ttcgcagttaatcctggcctgttagaaacatcagaaggctgtagacaaatactgggacagctacaaccatcccttca
gacaggatcagaagaacttagatcattatataatacagtagcaaccctctattgtgtgcatcaaaggatagagataa
aagacaccaaggaagctttagacaagatagaggaagagcaaaacaaaagtaagaccaccgcacagcaagcggccgct
gatcttcagacctggaggaggagatatgagggacaattggagaagtgaattatataaatataaagtagtaaaaattg
aaccattaggagtagcacccaccaaggcaaagagaagagtggtgcagagagaaaaaagagcagtgggaataggagct
ttgttccttgggttcttgggagcagcaggaagcactatgggcgcagcgtcaatgacgctgacggtacaggccagaca
attattgtctggtatagtgcagcagcagaacaatttgctgagggctattgaggcgcaacagcatctgttgcaactca
cagtctggggcatcaagcagctccaggcaagaatcctggctgtggaaagatacctaaaggatcaacagctcctgggg
atttggggttgctctggaaaactcatttgcaccactgctgtgccttggaatgctagttggagtaataaatctctgga
acagatttggaatcacacgacctggatggagtgggacagagaaattaacaattacacaagcttaatacactccttaa
ttgaagaatcgcaaaaccagcaagaaaagaatgaacaagaattattggaattagataaatgggcaagtttgtggaat
tggtttaacataacaaattggctgtggtatataaaattattcataatgatagtaggaggcttggtaggtttaagaat
agtttttgctgtactttctatagtgaatagagttaggcagggatattcaccattatcgtttcagacccacctcccaa
ccccgaggggacccgacaggcccgaaggaatagaagaagaaggtggagagagagacagagacagatccattcgatta
gtgaacggatcggcactgcgtgcgccaattctgcagacaaatggcagtattcatccacaattttaaaagaaaagggg
ggattggggggtacagtgcaggggaaagaatagtagacataatagcaacagacatacaaactaaagaattacaaaaa
caaattacaaaaattcaaaattttcgggtttattacagggacagcagagatccagtttggttaattaataacttcgt
atagcatacattatacgaagttattaaaaaaaaagcaccgactcggtgccactttttcaagttgataacggactagc
cttattttaacttgctatttctagctctaaaacCCACCGGCGCTCGCGCTACCggtgtttcgtcctttccacaagat
atataaagccaagaaatcgaaatactttcaagttacggtaagcatatgatagtccattttaaaacataattttaaaa
ctgcaaactacccaagaaattattactttctacgtcacgtattttgtactaatatctttgtgtttacagtcaaatta
attccaattatctctctaacagccttgtatcgtatatgcaaatatgaaggaatcatgggaaataggccctcttaatt
aattaacccgtgtcggctccagatctggcctccgcgccgggttttggcgcctcccgcgggcgcccccctcctcacgg
cgagcgctgccacgtcagacgaagggcgcagcgagcgtcctgatccttccgcccggacgctcaggacagcggcccgc
tgctcataagactcggccttagaaccccagtatcagcagaaggacattttaggacgggacttgggtgactctagggc
actggttttctttccagagagcggaacaggcgaggaaaagtagtcccttctcggcgattctgcggagggatctccgt
ggggcggtgaacgccgatgattatataaggacgcgccgggtgtggcacagctagttccgtcgcagccgggatttggg
tcgcggttcttgtttgtggatcgctgtgatcgtcacttggtgagtagcgggctgctgggctggccggggctttcgtg
gccgccgggccgctcggtgggacggaagcgtgtggagagaccgccaagggctgtagtctgggtccgcgagcaaggtt
gccctgaactgggggttggggggagcgcagcaaaatggcggctgttcccgagtcttgaatggaagacgcttgtgagg
cgggctgtgaggtcgttgaaacaaggtggggggcatggtgggcggcaagaacccaaggtcttgaggccttcgctaat
gcgggaaagctcttattcgggtgagatgggctggggcaccatctggggaccctgacgtgaagtttgtcactgactgg
agaactcggtttgtcgtctgttgcgggggcggcagttatggcg

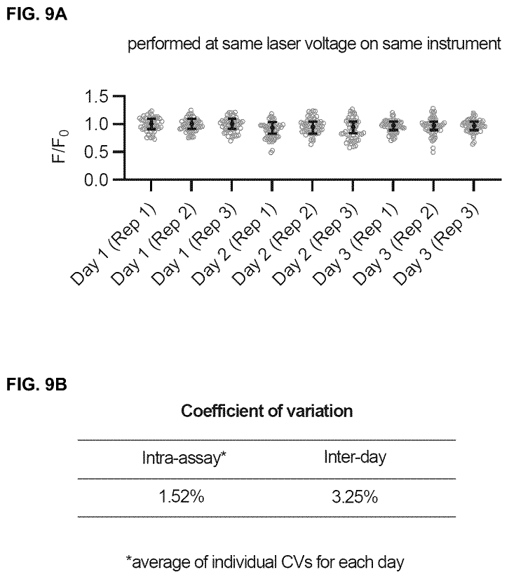

A Non-Limiting Example of a Temporal Polyamine Reporter (pLenti SFFV-DHFR-NLS-mCherry-OAZ1-miRFP670_2-WPRE, See FIGS. 1K-1L and 9A-9B)

(SEQ ID NO: 19)
atgagcacttttaaagttctgctatgtggcgcggtattatcccgtattgacgccgggcaagagcaactcggtcgccg
catacactattctcagaatgacttggttgagtactcaccagtcacagaaaagcatcttacggatggcatgacagtaa
gagaattatgcagtgctgccataaccatgagtgataacactgcggccaacttacttctgacaacgatcggaggaccg
aaggagctaaccgcttttttgcacaacatgggggatcatgtaactcgccttgatcgttgggaaccggagctgaatga
agccataccaaacgacgagcgtgacaccacgatgcctgtagcaatggcaacaacgttgcgcaaactattaactggcg
aactacttactctagcttcccggcaacaattaatagactggatggaggcggataaagttgcaggaccacttctgcgc
tcggcccttccggctggctggtttattgctgataaatctggagccggtgagcgtgggtctcgcggtatcattgcagc
actggggccagatggtaagccctcccgtatcgtagttatctacacgacggggagtcaggcaactatggatgaacgaa
atagacagatcgctgagataggtgcctcactgattaagcattggtaactgtcagaccaagtttactcatatatactt
tagattgatttaaaacttcatttttaatttaaaaggatctaggtgaagatcctttttgataatctcatgaccaaaat
cccttaacgtgagttttcgttccactgagcgtcagaccccgtagaaaagatcaaaggatcttcttgagatccttttt
ttctgcgcgtaatctgctgcttgcaaacaaaaaaaccaccgctaccagcggtggtttgtttgccggatcaagagcta
ccaactctttttccgaaggtaactggcttcagcagagcgcagataccaaatactgtccttctagtgtagccgtagtt
aggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacg
gggggttcgtgcacacagcccagcttggagcgaacgacctacaccgaactgagatacctacagcgtgagctatgaga
aagcgccacgcttcccgaagggagaaaggcggacaggtatccggtaagcggcagggtcggaacaggagagcgcacga
gggagcttccagggggaaacgcctggtatctttatagtcctgtcgggtttcgccacctctgacttgagcgtcgattt
ttgtgatgctcgtcaggggggcggagcctatggaaaaacgccagcaacgcggcctttttacggttcctggccttttg
ctggccttttgctcacatgttctttcctgcgttatcccctgattctgtggataaccgtattaccgcctttgagtgag
ctgataccgctcgccgcagccgaacgaccgagcgcagcgagtcagtgagcgaggaagcggaagagcgcccaatacgc
aaaccgcctctccccgcgcgttggccgattcattaatgcagctgtggaatgtgtgtcagttagggtgtggaaagtcc
ccaggctccccagcaggcagaagtatgcaaagcatgcatctcaattagtcagcaaccaggtgtggaaagtccccagg
ctccccagcaggcagaagtatgcaaagcatgcatctcaattagtcagcaaccatagtcccgcccctaactccgccca
tcccgcccctaactccgcccagttccgcccattctccgccccatggctgactaattttttttatttatgcagaggcc
gaggccgcctcggcctctgagctattccagaagtagtgaggaggcttttttggaggcctaggcttttgcaaaaagct
tggacacaagacaggcttgcgagatatgtttgagaataccactttatcccgcgtcagggagaggcagtgcgtaaaaa
gacgcggactcatgtgaaatactggtttttagtgcgccagatctctataatctcgcgcaacctattttcccctcgaa
cactttttaagccgtagataaacaggctgggacacttcacatgagcgaaaaatacatcgtcacctgggacatgttgc
agatccatgcacgtaaactcgcaagccgactgatgccttctgaacaatggaaaggcattattgccgtaagccgtggc
ggtctgtaccgggtgcgttactggcgcgtgaactgggtattcgtcatgtcgataccgtttgtatttccagctacgat
cacgacaaccagcgcgagcttaaagtgctgaaacgcgcagaaggcgatggcgaaggcttcatcgttattgatgacct
ggtggataccggtggtactgcggttgcgattcgtgaaatgtatccaaaagcgcactttgtcaccatcttcgcaaaac
cggctggtcgtccgctggttgatgactatgttgttgatatcccgcaagatacctggattgaacagccgtgggatatg
ggcgtcgtattcgtcccgccaatctccggtcgctaatcttttcaacgcctggcactgccgggcgttgttctttttaa
cttcaggcgggttacaatagtttccagtaagtattctggaggctgcatccatgacacaggcaaacctgagcgaaacc
ctgttcaaaccccgctttaaacatcctgaaacctcgacgctagtccgccgctttaatcacggcgcacaaccgcctgt
gcagtcggcccttgatggtaaaaccatccctcactggtatcgcatgattaaccgtctgatgtggatctggcgcggca
ttgacccacgcgaaatcctcgacgtccaggcacgtattgtgatgagcgatgccgaacgtaccgacgatgatttatac
gatacggtgattggctaccgtggcggcaactggatttatgagtgggccccggatctttgtgaaggaaccttacttct
gtggtgtgacataattggacaaactacctacagagatttaaagctctaaggtaaatataaaatttttaagtgtataa
tgtgttaaactactgattctaattgtttgtgtattttagattccaacctatggaactgatgaatgggagcagtggtg
gaatgcctttaatgaggaaaacctgttttgctcagaagaaatgccatctagtgatgatgaggctactgctgactctc
aacattctactcctccaaaaaagaagagaaaggtagaagaccccaaggactttccttcagaattgctaagttttttg
agtcatgctgtgtttagtaatagaactcttgcttgctttgctatttacaccacaaaggaaaaagctgcactgctata
caagaaaattatggaaaaatattctgtaacctttataagtaggcataacagttataatcataacatactgttttttc
ttactccacacaggcatagagtgtctgctattaataactatgctcaaaaattgtgtacctttagctttttaatttgt
aaaggggttaataaggaatatttgatgtatagtgccttgactagagatcataatcagccataccacatttgtagagg
ttttacttgctttaaaaaacctcccacacctccccctgaacctgaaacataaaatgaatgcaattgttgttgttaac
ttgtttattgcagcttataatggttacaaataaagcaatagcatcacaaatttcacaaataaagcatttttttcact
gcattctagttgtggtttgtccaaactcatcaatgtatcttatcatgtctggatcaactggataactcaagctaacc
aaaatcatcccaaacttcccaccccataccctattaccactgccaattacctagtggtttcatttactctaaacctg
tgattcctctgaattattttcattttaaagaaattgtatttgttaaatatgtactacaaacttagtagttggaaggg
ctaattcactcccaaagaagacaagatatccttgatctgtggatctaccacacacaaggctacttccctgattagca
gaactacacaccagggccaggggtcagatatccactgacctttggatggtgctacaagctagtaccagttgagccag
ataaggtagaagaggccaataaaggagagaacaccagcttgttacaccctgtgagcctgcatgggatggatgacccg
gagagagaagtgttagagtggaggtttgacagccgcctagcatttcatcacgtggcccgagagctgcatccggagta
cttcaagaactgctgatatcgagcttgctacaagggactttccgctggggactttccagggaggcgtggcctgggcg
ggactggggagtggcgagccctcagatcctgcatataagcagctgctttttgcctgtactgggtctctctggttaga
ccagatctgagcctgggagctctctggctaactagggaacccactgcttaagcctcaataaagcttgccttgagtgc
ttcaagtagtgtgtgcccgtctgttgtgtgactctggtaactagagatccctcagacccttttagtcagtgtggaaa
atctctagcagtggcgcccgaacagggacttgaaagcgaaagggaaaccagaggagctctctcgacgcaggactcgg
cttgctgaagcgcgcacggcaagaggcgaggggcggcgactggtgagtacgccaaaaattttgactagcggaggcta
gaaggagagagatgggtgcgagagcgtcagtattaagcgggggagaattagatcgcgatgggaaaaaattcggttaa
ggccagggggaaagaaaaaatataaattaaaacatatagtatgggcaagcagggagctagaacgattcgcagttaat
cctggcctgttagaaacatcagaaggctgtagacaaatactgggacagctacaaccatcccttcagacaggatcaga
agaacttagatcattatataatacagtagcaaccctctattgtgtgcatcaaaggatagagataaaagacaccaagg
aagctttagacaagatagaggaagagcaaaacaaaagtaagaccaccgcacagcaagcggccggtgatcttcagacc
tggacgatatatatgagggacaattggagaagtgaattatataaatataaagtagtaaaaattgaaccattaggagt
agcacccaccaaggcaaagagaagagtggtgcagagagaaaaaagagcagtgggaataggagctttgttccttgggt
tcttgggagcagcaggaagcactatgggcgcagcgtcaatgacgctgacggtacaggccagacaattattgtctggt
atagtgcagcagcagaacaatttgctgagggctattgaggcgcaacagcatctgttgcaactcacagtctggggcat
caagcagctccaggcaagaatcctggctgtggaaagatacctaaaggatcaacagctcctggggatttggggttgct
ctggaaaactcatttgcaccactgctgtgccttggaatgctagttggagtaataaatctctggaacagatttggaat
cacacgacctggatggagtgggacagagaaattaacaattacacaagcttaatacactccttaattgaagaatcgca
aaaccagcaagaaaagaatgaacaagaattattggaattagataaatgggcaagtttgtggaattggtttaacataa
caaattggctgtggtatataaaattattcataatgatagtaggaggcttggtaggtttaagaatagtttttgctgta
ctttctatagtgaatagagttaggcagggatattcaccattatcgtttcagacccacctcccaaccccgaggggacc
cgacaggcccgaaggaatagaagaagaaggtggagagagagacagagacagatccattcgattagtgaacggatctc
gacggtcgccaaatggcagtattcatccacaattttaaaagaaaaggggggattggggggtacagtgcaggggaaag
aatagtagacataatagcaacagacatacaaactaaagaattacaaaaacaaattacaaaaattcaaaattttcggg
tttattacagggacagcagagatccagtttggatcgataagcttgatatcgaattcctgcagccccgataaaataaa
agattttatttagtctccagaaaaaggggggaatgaaagaccccacctgtaggtttggcaagctagctgcagtaacg
ccattttgcaaggcatggaaaaataccaaaccaagaatagagaagttcagatcaagggcgggtacatgaaaatagct
aacgttgggccaaacaggatatctgcggtgagcagtttcggccccggcccggggccaagaacagatggtcaccgcag
tttcggccccggcccgaggccaagaacagatggtccccagatatggcccaaccctcagcagtttcttaagacccatc
agatgtttccaggctcccccaaggacctgaaatgaccctgcgccttatttgaattaaccaatcagcctgcttctcgc
ttctgttcgcgcgcttctgcttcccgagctctataaaagagctcacaacccctcactcggcgcgccagtcctccgac
agactgagtcgcccgggggggatctggagctctcgagaattctcAGCCACCATGATCAGTCTGATTGCggcgttagc
ggtagattacgttatcggcatggaaaacgccatgccgtggaacctgcctgccgatctcgcctggtttaaacgcaaca
ccttaaataaacccgtgattatgggccgccatacctgggaatcaatcggtcgtccgttgccaggacgcaaaaatatt
atcctcagcagtcaaccgagtacggacgatcgcgtaacgtgggtgaagtcggtggatgaagccatcgcggcgtgtgg
tgacgtaccagaaatcatggtgattggcggcggtcgcgttattgaacagttcttgccaaaagcgcaaaaactgtatc
tgacgcatatcgacgcagaagtggaaggcgacacccatttcccggattacgagccggatgactgggaatcggtattc
agcgaattccacgatgctgatgcgcagaactctcacagctattgctttGAGATTCTGGAGCGGCGAggctcgagcCC
CAAGAAGAAGCGCAAGGtgggaggaggcggaactagtGTGAGCAAGGGCGAGGAGGATAACATGGCCATCATCAAGG
AGTTCATGCGCTTCAAGGTGCACATGGAGGGCTCCGTGAACGGCCACGAGTTCGAGATCGAGGGCGAGGGCGAGGGC
CGCCCCTACGAGGGCACCCAGACCGCCAAGCTGAAGGTGACCAAGGGTGGCCCCCTGCCCTTCGCCTGGGACATCCT
GTCCCCTCAGTTCATGTACGGCTCCAAGGCCTACGTGAAGCACCCCGCCGACATCCCCGACTACTTGAAGCTGTCCT
TCCCCGAGGGCTTCAAGTGGGAGCGCGTGATGAACTTCGAGGACGGCGGCGTGGTGACCGTGACCCAGGACTCCTCC
CTGCAGGACGGCGAGTTCATCTACAAGGTGAAGCTGCGCGGCACCAACTTCCCCTCCGACGGCCCCGTAATGCAGAA
GAAGACCATGGGCTGGGAGGCCTCCTCCGAGCGGATGTACCCCGAGGACGGCGCCCTGAAGGGCGAGATCAAGCAGA
GGCTGAAGCTGAAGGACGGCGGCCACTACGACGCTGAGGTCAAGACCACCTACAAGGCCAAGAAGCCCGTGCAGCTG
CCCGGCGCCTACAACGTCAACATCAAGTTGGACATCACCTCCCACAACGAGGACTACACCATCGTGGAACAGTACGA
ACGCGCCGAGGGCCGCCACTCCACCGGCGGCATGGACGAGCTGTACAAGGGCTCCGGAGGTTTAATTAAcACTAGTC
TCCTAAGCCTGCACAGCCGCGGCGGCAGCAGCAGTGAGAGTTCCAGGGTCTCCCTCCACTGCTGTAGTAACCCGGGT
CCGGGGCCTCGGTGGTGCTCCTGATGCCCCTCACCCACCCCTGAAGATCCCAGGTGGGCGAGGGAATAGTCAGAGGG
ATCACAATCTTTCAGGATCCGCGCGTAAGGTCGATCTCACctcctgcgatcgcgagccgatccacatccccggcagc
attcagccgtgcggctgcctgctagcctgcgacgcgcaggcggtgcggatcacgcgcattacggaaaatgccggcgc
gttctttggacgcgaaactccgcgggtcggtgagctactcgccgattacttcggcgagaccgaagcccatgcgctgc
gcaacgcactggcgcagtcctccgatccaaagcgaccggcgctgatcttcggttggcgcgacggcctgaccggccgc
accttcgacatctcactgcatcgccatgacggtacatcgatcatcgagttcgagcctgcggcggccgaacaggccga
caatccgctgcggctgacgcggcagatcatcgcgcgcaccaaagaactgaagtcgctcgaagagatggccgcacggg
tgccgcgctatctgcaggcgatgctcggctatcaccgcgtgatgttgtaccgcttcgcggacgacggctccgggatg
gtgatcggcgaggcgaagcgcagcgacctcgagagctttctcggtcagcactttccggcgtcgctggtcccgcagca
ggcgcggctactgtacttgaagaacgcgatccgcgtggtctcggattcgcgcggcatcagcagccggatcgtgcccg
agcacgacgcctccggcgccgcgctcgatctgtcgttcgcgcacctgcgcagcatctcgccctgccatctcgaattt
ctgcggaacatgggcgtcagcgcctcgatgtcgctgtcgatcatcattgacggcacgctatggggattgatcatctg
tcatcattacgagccgcgtgccgtgccgatggcgcagcgcgtcgcggccaaaaggttcgccgagcgcttatcgacgc
acttcaccgccGCCCACCACCAACGCtaaGCGGCCGCCTGACGTAACTAGTGTAAGGgcgactctagagtcgacctg
caggcatgcaagcttgatatcaagcttatcgataatcaacctctggattacaaaatttgtgaaagattgactggtat
tcttaactatgttgctccttttacgctatgtggatacgctgctttaatgcctttgtatcatgctattgcttcccgta
tggctttcattttctcctccttgtataaatcctggttgctgtctctttatgaggagttgtggcccgttgtcaggcaa
cgtggcgtggtgtgcactgtgtttgctgacgcaacccccactggttggggcattgccaccacctgtcagctcctttc
cgggactttcgctttccccctccctattgccacggcggaactcatcgccgcctgccttgcccgctgctggacagggg
ctcggctgttgggcactgacaattccgtggtgttgtcggggaaatcatcgtcctttccttggctgctcgcctgtgtt
gccacctggattctgcgcgggacgtccttctgctacgtcccttcggccctcaatccagcggaccttccttcccgcgg
cctgctgccggctctgcggcctcttccgcgtcttcgccttcgccctcagacgagtcggatctccctttgggccgcct
ccccgcatcgataccgtcgacctcgagggaattaattcgagctcggtacctttaagaccaatgacttacaaggcagc
tgtagatcttagccactttttaaaagaaaaggggggactggaagggctaattcactcccaacgaagacaagatctgc
tttttgcttgtactgggtctctctggttagaccagatctgagcctgggagctctctggctaactagggaacccactg
cttaagcctcaataaagcttgccttgagtgcttcaagtagtgtgtgcccgtctgttgtgtgactctggtaactagag
atccctcagacccttttagtcagtgtggaaaatctctagcagcatctagaattaattccgtgtattctatagtgtca
cctaaatcgtatgtgtatgatacataaggttatgtattaattgtagccgcgttctaacgacaatatgtacaagccta
attgtgtagcatctggcttactgaagcagaccctatcatctctctcgtaaactgccgtcagagtcggtttggttgga
cgaaccttctgagtttctggtaacgccgtcccgcacccggaaatggtcagcgaaccaatcagcagggtcatcgctag
ccagatcctctacgccggacgcatcgtggccggcatcaccggcgccacaggtgcggttgctggcgcctatatcgccg
acatcaccgatggggaagatcgggctcgccacttcgggctcatgagcgcttgtttcggcgtgggtatggtggcaggc
cccgtggccgggggactgttgggcgccatctccttgcatgcaccattccttgcggcggcggtgctcaacggcctcaa
cctactactgggctgcttcctaatgcaggagtcgcataagggagagcgtcgaatggtgcactctcagtacaatctag
ctctgatgccgcatagttaagccagccccgacacccgccaacacccgctgacgcgccctgacgggcttgtctgctcc
cggcatccgcttacagacaagctgtgaccgtctccgggagctgcatgtgtcagaggttttcaccgtcatcaccgaaa
cgcgcgagacgaaagggcctcgtgatacgcctatttttataggttaatgtcatgataataatggtttcttagacgtc
aggtggcacttttcggggaaatgtgcgcggaacccctatttgtttatttttctaaatacattcaaatatgtatccgc
tcatgagacaataaccctgataaatgcttcaataatattgaaaaaggaagagtatgagtattcaacatttccgtgtc
gcccttattcccttttttgcggcattttgccttcctgtttttgctcacccagaaacgctggtgaaagtaaaagatgc
tgaagatcagttgggtgcacgagtgggttacatcgaactggatctcaacagcggtaagatccttgagagttttcgcc
ccgaagaacgttttccaatg

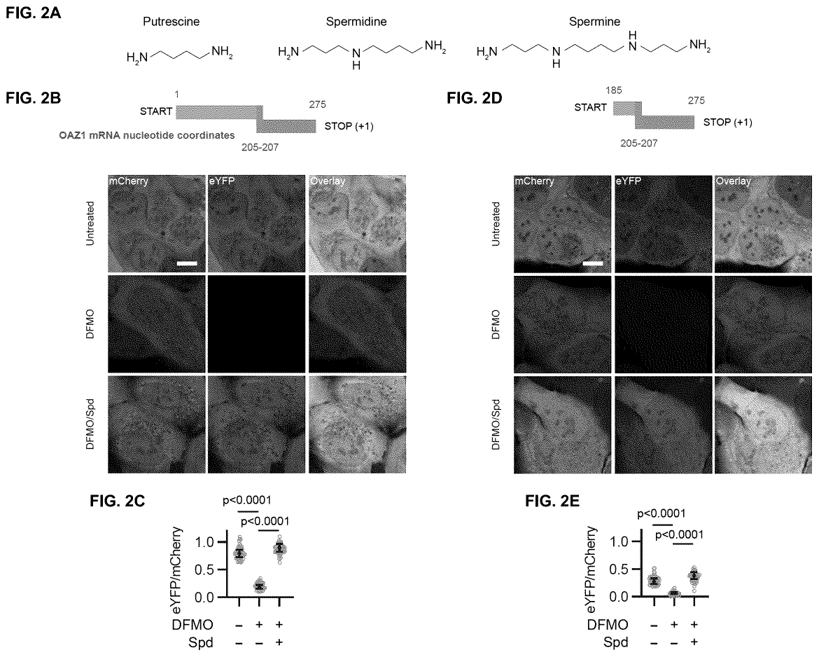

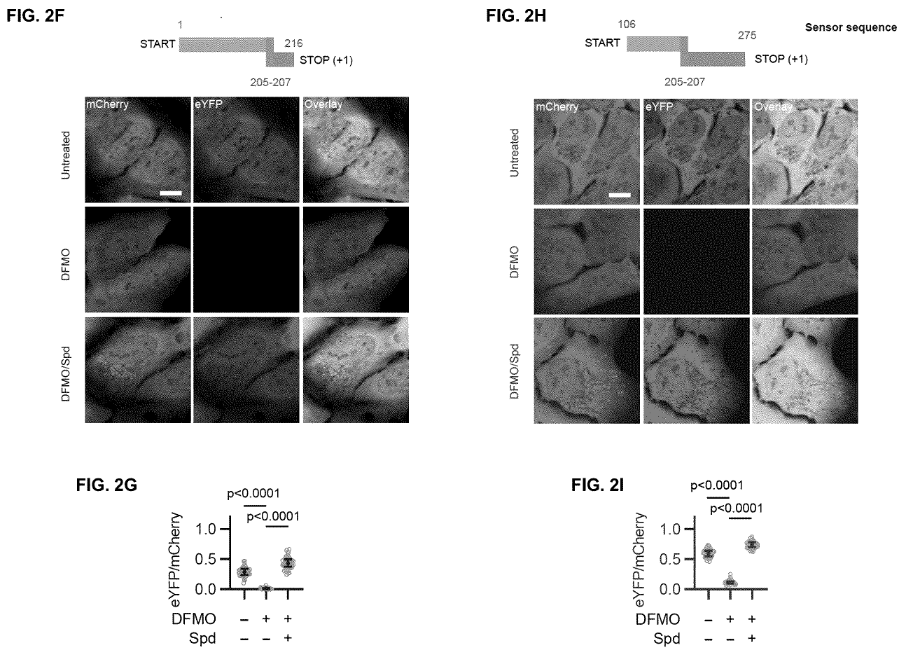

OAZ1 (1-275) (pHR-Tre3G-mCherry-OAZ1 FS-eYFP, See FIG. 2B)

(SEQ ID NO: 20)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTGTGAAATCCTCCCTGCAGCGGATCCTCAATAGCCACTGCTTCGCCAGAGAGAAGGAAGGGGATAAACCCA
GCGCCACCATCCACGCCAGCCGCACCATGCCGCTCCTAAGCCTGCACAGCCGCGGCGGCAGCAGCAGTGAGAGTTCC
AGGGTCTCCCTCCACTGCTGTAGTAACCCGGGTCCGGGGCCTCGGTGGTGCTCCTGATGCCCCTCACCCACCCCTGA
AGATCCCAGGTGGGCGAGGGAATAGTCAGAGGGATCACAATCTTTCAGGCGTGAGCAAGGGCGAGGAGCTGTTCACC
GGGGTGGTGCCCATCCTGGTCGAGCTGGACGGCGACGTAAACGGCCACAAGTTCAGCGTGTCCGGCGAGGGCGAGGG
CGATGCCACCTACGGCAAGCTGACCCTGAAGTTCATCTGCACCACCGGCAAGCTGCCCGTGCCCTGGCCCACCCTCG
TGACCACCTTCGGCTACGGCCTGCAGTGCTTCGCCCGCTACCCCGACCACATGAAGCAGCACGACTTCTTCAAGTCC
GCCATGCCCGAAGGCTACGTCCAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCAAGGAGGACGGCAACATCCTGG
GGCACAAGCTGGAGTACAACTACAACAGCCACAACGTCTATATCATGGCCGACAAGCAGAAGAACGGCATCAAGGTG
AACTTCAAGATCCGCCACAACATCGAGGACGGCAGCGTGCAGCTCGCCGACCACTACCAGCAGAACACCCCCATCGG
CGACGGCCCCGTGCTGCTGCCCGACAACCACTACCTGAGCTACCAGTCCAAACTGAGCAAAGACCCCAACGAGAAGC
GCGATCACATGGTCCTGCTGGAGTTCGTGACCGCCGCCGGGATCACTCTCGGCATGGACGAGCTGTACAAGTGATGA
ATTgcGGCCGCCTGACGTAACTAGTgTAAggcatgcaagcttgatatcaagcttatcgataatcaacctctggatta
caaaatttgtgaaagattgactggtattcttaactatgttgctccttttacgctatgtggatacgctgctttaatgc
ctttgtatcatgctattgcttcccgtatggctttcattttctcctccttgtataaatcctggttgctgtctctttat
gaggagttgtggcccgttgtcaggcaacgtggcgtggtgtgcactgtgtttgctgacgcaacccccactggttgggg
cattgccaccacctgtcagctcctttccgggactttcgctttccccctccctattgccacggcggaactcatcgccg
cctgccttgcccgctgctggacaggggctcggctgttgggcactgacaattccgtggtgttgtcggggaaatcatcg
tcctttccttggctgctcgcctgtgttgccacctggattctgcgcgggacgtccttctgctacgtcccttcggccct
caatccagcggaccttccttcccgcggcctgctgccggctctgcggcctcttccgcgtcttcgccttcgccctcaga
cgagtcggatctccctttgggccgcctccccgcat

OAZ1 (185-275) (pHR-Tre3G-mCherry-OAZ1 FS-eYFP, See FIG. 2C)

(SEQ ID NO: 21)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTCCGGGGCCTCGGTGGTGCTCCTGATGCCCCTCACCCACCCCTGAAGATCCCAGGTGGGCGAGGGAATAGT
CAGAGGGATCACAATCTTTCAGGCGTGAGCAAGGGCGAGGAGCTGTTCACCGGGGTGGTGCCCATCCTGGTCGAGCT
GGACGGCGACGTAAACGGCCACAAGTTCAGCGTGTCCGGCGAGGGCGAGGGCGATGCCACCTACGGCAAGCTGACCC
TGAAGTTCATCTGCACCACCGGCAAGCTGCCCGTGCCCTGGCCCACCCTCGTGACCACCTTCGGCTACGGCCTGCAG
TGCTTCGCCCGCTACCCCGACCACATGAAGCAGCACGACTTCTTCAAGTCCGCCATGCCCGAAGGCTACGTCCAGGA
GCGCACCATCTTCTTCAAGGACGACGGCAACTACAAGACCCGCGCCGAGGTGAAGTTCGAGGGCGACACCCTGGTGA
ACCGCATCGAGCTGAAGGGCATCGACTTCAAGGAGGACGGCAACATCCTGGGGCACAAGCTGGAGTACAACTACAAC
AGCCACAACGTCTATATCATGGCCGACAAGCAGAAGAACGGCATCAAGGTGAACTTCAAGATCCGCCACAACATCGA
GGACGGCAGCGTGCAGCTCGCCGACCACTACCAGCAGAACACCCCCATCGGCGACGGCCCCGTGCTGCTGCCCGACA
ACCACTACCTGAGCTACCAGTCCAAACTGAGCAAAGACCCCAACGAGAAGCGCGATCACATGGTCCTGCTGGAGTTC
GTGACCGCCGCCGGGATCACTCTCGGCATGGACGAGCTGTACAAGTGATGAATTgcGGCCGCCTGACGTAACTAGTg
TAAggcatgcaagcttgatatcaagcttatcgataatcaacctctggattacaaaatttgtgaaagattgactggta
ttcttaactatgttgctccttttacgctatgtggatacgctgctttaatgcctttgtatcatgctattgcttcccgt
atggctttcattttctcctccttgtataaatcctggttgctgtctctttatgaggagttgtggcccgttgtcaggca
acgtggcgtggtgtgcactgtgtttgctgacgcaacccccactggttggggcattgccaccacctgtcagctccttt
ccgggactttcgctttccccctccctattgccacggcggaactcatcgccgcctgccttgcccgctgctggacaggg
gctcggctgttgggcactgacaattccgtggtgttgtcggggaaatcatcgtcctttccttggctgctcgcctgtgt
tgccacctggattctgcgcgggacgtccttctgctacgtcccttcggccctcaatccagcggaccttccttcccgcg
gcctgctgccggctctgcggcctcttccgcgtcttcgccttcgccctcagacgagtcggatctccctttgggccgcc
tccccgcat

OAZ1 (1-216) (pHR-Tre3G-mCherry-OAZ1 FS-eYFP, See FIG. 2D)

(SEQ ID NO: 22)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTGTGAAATCCTCCCTGCAGCGGATCCTCAATAGCCACTGCTTCGCCAGAGAGAAGGAAGGGGATAAACCCA
GCGCCACCATCCACGCCAGCCGCACCATGCCGCTCCTAAGCCTGCACAGCCGCGGCGGCAGCAGCAGTGAGAGTTCC
AGGGTCTCCCTCCACTGCTGTAGTAACCCGGGTCCGGGGCCTCGGTGGTGCTCCTGATGCCCCTCACGTGAGCAAGG
GCGAGGAGCTGTTCACCGGGGTGGTGCCCATCCTGGTCGAGCTGGACGGCGACGTAAACGGCCACAAGTTCAGCGTG
TCCGGCGAGGGCGAGGGCGATGCCACCTACGGCAAGCTGACCCTGAAGTTCATCTGCACCACCGGCAAGCTGCCCGT
GCCCTGGCCCACCCTCGTGACCACCTTCGGCTACGGCCTGCAGTGCTTCGCCCGCTACCCCGACCACATGAAGCAGC
ACGACTTCTTCAAGTCCGCCATGCCCGAAGGCTACGTCCAGGAGCGCACCATCTTCTTCAAGGACGACGGCAACTAC
AAGACCCGCGCCGAGGTGAAGTTCGAGGGCGACACCCTGGTGAACCGCATCGAGCTGAAGGGCATCGACTTCAAGGA
GGACGGCAACATCCTGGGGCACAAGCTGGAGTACAACTACAACAGCCACAACGTCTATATCATGGCCGACAAGCAGA
AGAACGGCATCAAGGTGAACTTCAAGATCCGCCACAACATCGAGGACGGCAGCGTGCAGCTCGCCGACCACTACCAG
CAGAACACCCCCATCGGCGACGGCCCCGTGCTGCTGCCCGACAACCACTACCTGAGCTACCAGTCCAAACTGAGCAA
AGACCCCAACGAGAAGCGCGATCACATGGTCCTGCTGGAGTTCGTGACCGCCGCCGGGATCACTCTCGGCATGGACG
AGCTGTACAAGTGATGAATTgcGGCCGCCTGACGTAACTAGTgTAAggcatgcaagcttgatatcaagcttatcgat
aatcaacctctggattacaaaatttgtgaaagattgactggtattcttaactatgttgctccttttacgctatgtgg
atacgctgctttaatgcctttgtatcatgctattgcttcccgtatggctttcattttctcctccttgtataaatcct
ggttgctgtctctttatgaggagttgtggcccgttgtcaggcaacgtggcgtggtgtgcactgtgtttgctgacgca
acccccactggttggggcattgccaccacctgtcagctcctttccgggactttcgctttccccctccctattgccac
ggcggaactcatcgccgcctgccttgcccgctgctggacaggggctcggctgttgggcactgacaattccgtggtgt
tgtcggggaaatcatcgtcctttccttggctgctcgcctgtgttgccacctggattctgcgcgggacgtccttctgc
tacgtcccttcggccctcaatccagcggaccttccttcccgcggcctgctgccggctctgcggcctcttccgcgtct
tcgccttcgccctcagacgagtcggatctccctttgggccgcctccccgcat

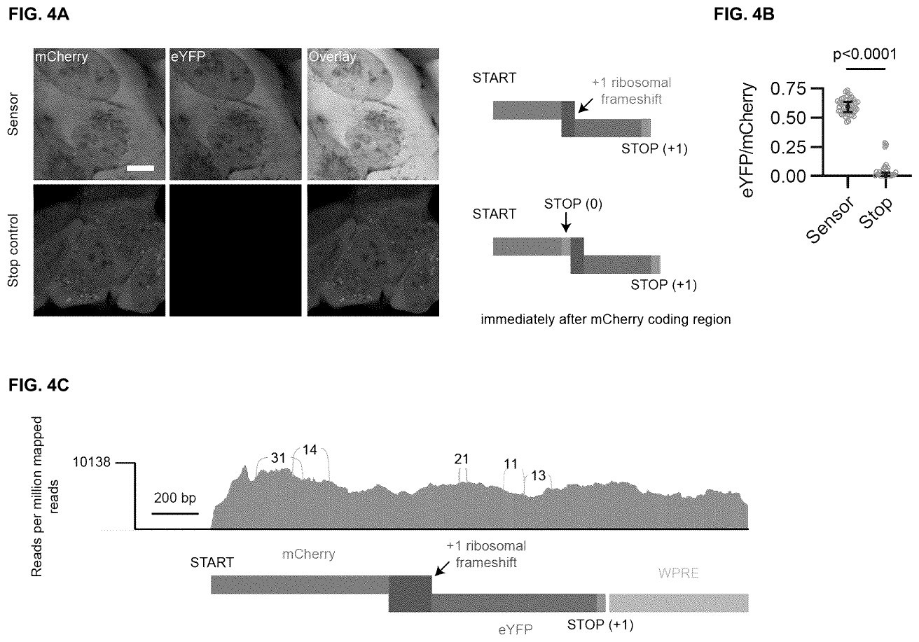

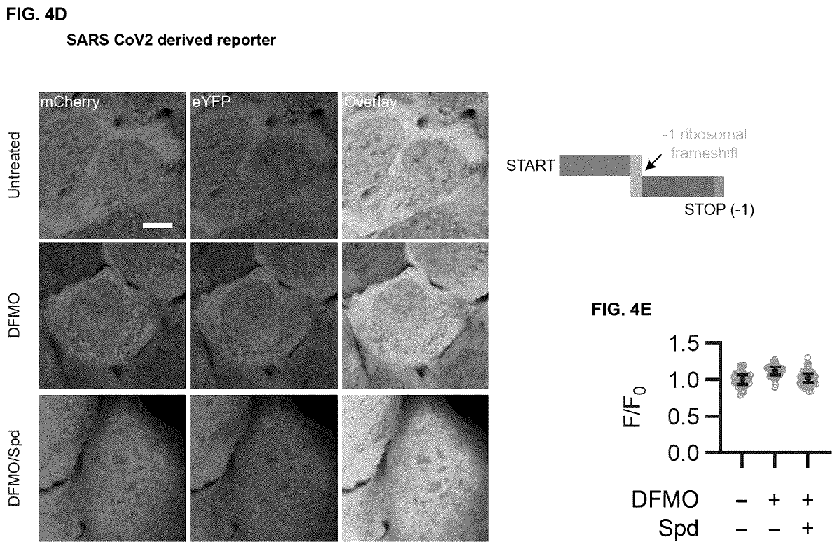

SARS Cov2-1 Frameshift Reporter (pHR-Tre3G-mCherry-SARS_Cov2 FS-eYFP, See FIGS. 4C-4D)

(SEQ ID NO: 23)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTAAccctgtgggttttacacttaaaaacacagtctgtaccgtctgcggtatgtggaaaggttatggctgta
gttgtgatcaactccgcgaacccatgcttcagtcagctgatgcacaatcgtttttaaacgggtttgcggtgtaagtg
cagcccgtcttacaccgtgcggcacaggcactagtactgatgtcgtatacagggcttttgacatctacaatgataaa
gtagctggttttgctaaattcctaaaaactaattgttgtcgcttccaagaaaaggacgaagatgacaatttaattga
tGGTGGTGGTGgatccGTGAGCAAGGGCGAGGAGCTGTTCACCGGGGTGGTGCCCATCCTGGTCGAGCTGGACGGCG
ACGTAAACGGCCACAAGTTCAGCGTGTCCGGCGAGGGCGAGGGCGATGCCACCTACGGCAAGCTGACCCTGAAGTTC
ATCTGCACCACCGGCAAGCTGCCCGTGCCCTGGCCCACCCTCGTGACCACCTTCGGCTACGGCCTGCAGTGCTTCGC
CCGCTACCCCGACCACATGAAGCAGCACGACTTCTTCAAGTCCGCCATGCCCGAAGGCTACGTCCAGGAGCGCACCA
TCTTCTTCAAGGACGACGGCAACTACAAGACCCGCGCCGAGGTGAAGTTCGAGGGCGACACCCTGGTGAACCGCATC
GAGCTGAAGGGCATCGACTTCAAGGAGGACGGCAACATCCTGGGGCACAAGCTGGAGTACAACTACAACAGCCACAA
CGTCTATATCATGGCCGACAAGCAGAAGAACGGCATCAAGGTGAACTTCAAGATCCGCCACAACATCGAGGACGGCA
GCGTGCAGCTCGCCGACCACTACCAGCAGAACACCCCCATCGGCGACGGCCCCGTGCTGCTGCCCGACAACCACTAC
CTGAGCTACCAGTCCAAACTGAGCAAAGACCCCAACGAGAAGCGCGATCACATGGTCCTGCTGGAGTTCGTGACCGC
CGCCGGGATCACTCTCGGCATGGACGAGCTGTACAAGTGATGAATTgcGGCCGCCTGACGTAACTAGTgTAAggcat
gcaagcttgatatcaagcttatcgataatcaacctctggattacaaaatttgtgaaagattgactggtattcttaac
tatgttgctccttttacgctatgtggatacgctgctttaatgcctttgtatcatgctattgcttcccgtatggcttt
cattttctcctccttgtataaatcctggttgctgtctctttatgaggagttgtggcccgttgtcaggcaacgtggcg
tggtgtgcactgtgtttgctgacgcaacccccactggttggggcattgccaccacctgtcagctcctttccgggact
ttcgctttccccctccctattgccacggcggaactcatcgccgcctgccttgcccgctgctggacaggggctcggct
gttgggcactgacaattccgtggtgttgtcggggaaatcatcgtcctttccttggctgctcgcctgtgttgccacct
ggattctgcgcgggacgtccttctgctacgtcccttcggccctcaatccagcggaccttccttcccgcggcctgctg
ccggctctgcggcctcttccgcgtcttcgccttcgccctcagacgagtcggatctccctttgggccgcctccccgca
t

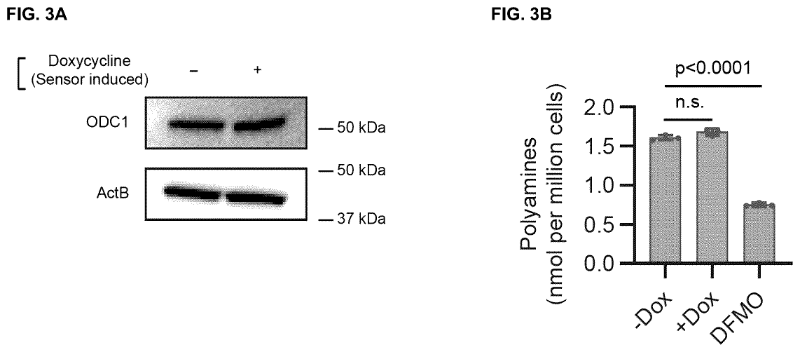

STOP(0) Control (pHR-Tre3G-mCherry-STOP-OAZ1 FS-eYFP, See FIGS. 3A-3B)

(SEQ ID NO: 24)
cgataccgtcgacctcgagggaattaattcgagctcggtacctttaagaccaatgacttacaaggcagctgtagatc
ttagccactttttaaaagaaaaggggggactggaagggctaattcactcccaacgaagacaagatctgctttttgct
tgtactgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctca
gacccttttagtcagtgtggaaaatctctagcagcatctagaattaattccgtgtattctatagtgtcacctaaatc
gtatgtgtatgatacataaggttatgtattaattgtagccgcgttctaacgacaatatgtacaagcctaattgtgta
gcatctggcttactgaagcagaccctatcatctctctcgtaaactgccgtcagagtcggtttggttggacgaacctt
ctgagtttctggtaacgccgtcccgcacccggaaatggtcagcgaaccaatcagcagggtcatcgctagccagatcc
tctacgccggacgcatcgtggccggcatcaccggcgccacaggtgcggttgctggcgcctatatcgccgacatcacc
gatggggaagatcgggctcgccacttcgggctcatgagcgcttgtttcggcgtgggtatggtggcaggccccgtggc
cgggggactgttgggcgccatctccttgcatgcaccattccttgcggcggcggtgctcaacggcctcaacctactac
tgggctgcttcctaatgcaggagtcgcataagggagagcgtcgaatggtgcactctcagtacaatctgctctgatgc
cgcatagttaagccagccccgacacccgccaacacccgctgacgcgccctgacgggcttgtctgctcccggcatccg
cttacagacaagctgtgaccgtctccgggagctgcatgtgtcagaggttttcaccgtcatcaccgaaacgcgcgaga
cgaaagggcctcgtgatacgcctatttttataggttaatgtcatgataataatggtttcttagacgtcaggtggcac
ttttcggggaaatgtgcgcggaacccctatttgtttatttttctaaatacattcaaatatgtatccgctcatgagac
aataaccctgataaatgcttcaataatattgaaaaaggaagagtatgagtattcaacatttccgtgtcgcccttatt
cccttttttgcggcattttgccttcctgtttttgctcacccagaaacgctggtgaaagtaaaagatgctgaagatca
gttgggtgcacgagtgggttacatcgaactggatctcaacagcggtaagatccttgagagttttcgccccgaagaac
gttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatgg
catgacagtaagagaattatgcagtgctgccataaccatgagtgataacactgcggccaacttacttctgacaacga
tcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggac
cacttctgcgctcggcccttccggctggctggtttattgctgataaatctggagccggtgagcgtgggtctcgcggt
atcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttact
catatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttg
agatcctttttttctgcgcgtaatctgctgcttgcaaacaaaaaaaccaccgctaccagcggtggtttgtttgccgg
atcaagagctaccaactctttttccgaaggtaactggcttcagcagagcgcagataccaaatactgtctttctagtg
tagccgtagttaggccaccacttcaagaactctgtagcaccgcctacatacctcgctctgctaatcctgttaccagt
ggctgctgccagtggcgataagtcgtgtcttaccgggttggactcaagacgatagttaccggataaggcgcagcggt
cgggctgaacggggggttcgtgcacacagcccagcttggagcgaacgacctacaccgaactgagatacctacagcgt
gagctatgagaaagcgccacgcttcccgaagggagaaaggcggacaggtatccggtaagcggcagggtcggaacagg
agagcgcacgagggagcttccagggggaaacgcctggtatctttatagtcctgtcgggtttcgccacctctgacttg
agcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgc
ctttgagtgagctgataccgctcgccgcagccgaacgaccgagcgcagcgagtcagtgagcgaggaagcggaagagc
gcccaatacgcaaaccgcctctccccgcgcgttggccgattcattaatgcagctgtggaatgtgtgtcagttagggt
gtggaaagtccccaggctccccagcaggcagaagtatgcaaagcatgcatctcaattagtcagcaaccaggtgtgga
aagtccccaggctccccagcaggcagaagtatgcaaagcatgcatctcaattagtcagcaaccatagtcccgcccct
aactccgcccatcccgcccctaactccgcccagttccgcccattctccgccccatggctgactaattttttttattt
atgcagaggccgaggccgcctcggcctctgagctattccagaagtagtgaggaggcttttttggaggcctaggcttt
tgcaaaaagcttggacacaagacaggcttgcgagatatgtttgagaataccactttatcccgcgtcagggagaggca
gtgcgtaaaaagacgcggactcatgtgaaatactggtttttagtgcgccagatctctataatctcgcgcaacctatt
ttcccctcgaacactttttaagccgtagataaacaggctgggacacttcacatgagcgaaaaatacatcgtcacctg
ggacatgttgcagatccatgcacgtaaactcgcaagccgactgatgccttctgaacaatggaaaggcattattgccg
taagccgtggcggtctgtaccgggtgcgttactggcgcgtgaactgggtattcgtcatgtcgataccgtttgtattt
ccagctacgatcacgacaaccagcgcgagcttaaagtgctgaaacgcgcagaaggcgatggcgaaggcttcatcgtt
attgatgacctggtggataccggtggtactgcggttgcgattcgtgaaatgtatccaaaagcgcactttgtcaccat
cttcgcaaaaccggctggtcgtccgctggttgatgactatgttgttgatatcccgcaagatacctggattgaacagc
cgtgggatatgggcgtcgtattcgtcccgccaatctccggtcgctaatcttttcaacgcctggcactgccgggcgtt
gttctttttaacttcaggcgggttacaatagtttccagtaagtattctggaggctgcatccatgacacaggcaaacc
tgagcgaaaccctgttcaaaccccgctttaaacatcctgaaacctcgacgctagtccgccgctttaatcacggcgca
caaccgcctgtgcagtcggcccttgatggtaaaaccatccctcactggtatcgcatgattaaccgtctgatgtggat
ctggcgcggcattgacccacgcgaaatcctcgacgtccaggcacgtattgtgatgagcgatgccgaacgtaccgacg
atgatttatacgatacggtgattggctaccgtggcggcaactggatttatgagtgggccccggatctttgtgaagga
accttacttctgtggtgtgacataattggacaaactacctacagagatttaaagctctaaggtaaatataaaatttt
taagtgtataatgtgttaaactactgattctaattgtttgtgtattttagattccaacctatggaactgatgaatgg
gagcagtggtggaatgcctttaatgaggaaaacctgttttgctcagaagaaatgccatctagtgatgatgaggctac
tgctgactctcaacattctactcctccaaaaaagaagagaaaggtagaagaccccaaggactttccttcagaattgc
taagttttttgagtcatgctgtgtttagtaatagaactcttgcttgctttgctatttacaccacaaaggaaaaagct
gcactgctatacaagaaaattatggaaaaatattctgtaacctttataagtaggcataacagttataatcataacat
actgttttttcttactccacacaggcatagagtgtctgctattaataactatgctcaaaaattgtgtacctttagct
ttttaatttgtaaaggggttaataaggaatatttgatgtatagtgccttgactagagatcataatcagccataccac
atttgtagaggttttacttgctttaaaaaacctcccacacctccccctgaacctgaaacataaaatgaatgcaattg
ttgttgttaacttgtttattgcagcttataatggttacaaataaagcaatagcatcacaaatttcacaaataaagca
tttttttcactgcattctagttgtggtttgtccaaactcatcaatgtatcttatcatgtctggatcaactggataac
tcaagctaaccaaaatcatcccaaacttcccaccccataccctattaccactgccaattacctagtggtttcattta
ctctaaacctgtgattcctctgaattattttcattttaaagaaattgtatttgttaaatatgtactacaaacttagt
agttggaagggctaattcactcccaaagaagacaagatatccttgatctgtggatctaccacacacaaggctacttc
cctgattagcagaactacacaccagggccaggggtcagatatccactgacctttggatggtgctacaagctagtacc
agttgagccagataaggtagaagaggccaataaaggagagaacaccagcttgttacaccctgtgagcctgcatggga
tggatgacccggagagagaagtgttagagtggaggtttgacagccgcctagcatttcatcacgtggcccgagagctg
catccggagtacttcaagaactgctgatatcgagcttgctacaagggactttccgctggggactttccagggaggcg
tggcctgggcgggactggggagtggcgagccctcagatcctgcatataagcagctgctttttgcctgtactgggtct
ctctggttagaccagatctgagcctgggagctctctggctaactagggaacccactgcttaagcctcaataaagctt
gccttgagtgcttcaagtagtgtgtgcccgtctgttgtgtgactctggtaactagagatccctcagacccttttagt
cagtgtggaaaatctctagcagtggcgcccgaacagggacttgaaagcgaaagggaaaccagaggagctctctcgac
gcaggactcggcttgctgaagcgcgcacggcaagaggcgaggggcggcgactggtgagtacgccaaaaattttgact
agcggaggctagaaggagagagatgggtgcgagagcgtcagtattaagcgggggagaattagatcgcgatgggaaaa
aattcggttaaggccagggggaaagaaaaaatataaattaaaacatatagtatgggcaagcagggagctagaacgat
tcgcagttaatcctggcctgttagaaacatcagaaggctgtagacaaatactgggacagctacaaccatcccttcag
acaggatcagaagaacttagatcattatataatacagtagcaaccctctattgtgtgcatcaaaggatagagataaa
agacaccaaggaagctttagacaagatagaggaagagcaaaacaaaagtaagaccaccgcacagcaagcggccggcc
gctgatcttcagacctggaggaggagatatgagggacaattggagaagtgaattatataaatataaagtagtaaaaa
ttgaaccattaggagtagcacccaccaaggcaaagagaagagtggtgcagagagaaaaaagagcagtgggaatagga
gctttgttccttgggttcttgggagcagcaggaagcactatgggcgcagcgtcaatgacgctgacggtacaggccag
acaattattgtctggtatagtgcagcagcagaacaatttgctgagggctattgaggcgcaacagcatctgttgcaac
tcacagtctggggcatcaagcagctccaggcaagaatcctggctgtgaaagatacctaaaggatcaacagctcctgg
ggatttggggttgctctggaaaactcatttgcaccactgctgtgccttggaatgctagttggagtaataaatctctg
gaacagatttggaatcacacgacctggatggagtgggacagagaaattaacaattacacaagcttaatacactcctt
aattgaagaatcgcaaaaccagcaagaaaagaatgaacaagaattattggaattagataaatgggcaagtttgtgga
attggtttaacataacaaattggctgtggtatataaaattattcataatgatagtaggaggcttggtaggtttaaga
atagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgat
tagtgaacggatctcgacggtatcgccaaatggcagtattcatccacaattttaaaagaaaaggggggattgggggg
tacagtgcaggggaaagaatagtagacataatagcaacagacatacaaactaaagaattacaaaaacaaattacaaa
aattcaaaattttcgggtttattacagggacagcagagatccagtttatcgatgaggccctttcgtcttcactcgag
tttactccctatcagtgatagagaacgtatgaagagtttactccctatcagtgatagagaacgtatgcagactttac
tccctatcagtgatagagaacgtataaggagtttactccctatcagtgatagagaacgtatgaccagtttactccct
atcagtgatagagaacgtatctacagtttactccctatcagtgatagagaacgtatatccagtttactccctatcag
tgatagagaacgtatgtcgaggtaggcgtgtacggtgggcgcctataaaagcagagctcgtttagtgaaccgtcaga
tcgcctggagcaattccacaacacttttgtcttatacttACGCGTGCCACCATGGTGAGCAAGGGCGAGGAGGATAA
CATGGCCATCATCAAGGAGTTCATGCGCTTCAAGGTGCACATGGAGGGCTCCGTGAACGGCCACGAGTTCGAGATCG
AGGGCGAGGGCGAGGGCCGCCCCTACGAGGGCACCCAGACCGCCAAGCTGAAGGTGACCAAGGGTGGCCCCCTGCCC
TTCGCCTGGGACATCCTGTCCCCTCAGTTCATGTACGGCTCCAAGGCCTACGTGAAGCACCCCGCCGACATCCCCGA
CTACTTGAAGCTGTCCTTCCCCGAGGGCTTCAAGTGGGAGCGCGTGATGAACTTCGAGGACGGCGGCGTGGTGACCG
TGACCCAGGACTCCTCCCTGCAGGACGGCGAGTTCATCTACAAGGTGAAGCTGCGCGGCACCAACTTCCCCTCCGAC
GGCCCCGTAATGCAGAAGAAGACCATGGGCTGGGAGGCCTCCTCCGAGCGGATGTACCCCGAGGACGGCGCCCTGAA
GGGCGAGATCAAGCAGAGGCTGAAGCTGAAGGACGGCGGCCACTACGACGCTGAGGTCAAGACCACCTACAAGGCCA
AGAAGCCCGTGCAGCTGCCCGGCGCCTACAACGTCAACATCAAGTTGGACATCACCTCCCACAACGAGGACTACACC
ATCGTGGAACAGTACGAACGCGCCGAGGGCCGCCACTCCACCGGCGGCATGGACGAGCTGTACAAGGGCTCCGGAGG
TTTAATTAAATAATAAACTAGTCTCCTAAGCCTGCACAGCCGCGGCGGCAGCAGCAGTGAGAGTTCCAGGGTCTCCC
TCCACTGCTGTAGTAACCCGGGTCCGGGGCCTCGGTGGTGCTCCTGATGCCCCTCACCCACCCCTGAAGATCCCAGG
TGGGCGAGGGAATAGTCAGAGGGATCACAATCTTTCAGgatccGTGAGCAAGGGCGAGGAGCTGTTCACCGGGGTGG
TGCCCATCCTGGTCGAGCTGGACGGCGACGTAAACGGCCACAAGTTCAGCGTGTCCGGCGAGGGCGAGGGCGATGCC
ACCTACGGCAAGCTGACCCTGAAGTTCATCTGCACCACCGGCAAGCTGCCCGTGCCCTGGCCCACCCTCGTGACCAC
CTTCGGCTACGGCCTGCAGTGCTTCGCCCGCTACCCCGACCACATGAAGCAGCACGACTTCTTCAAGTCCGCCATGC
CCGAAGGCTACGTCCAGGAGCGCACCATCTTCTTCAAGGACGACGGCAACTACAAGACCCGCGCCGAGGTGAAGTTC
GAGGGCGACACCCTGGTGAACCGCATCGAGCTGAAGGGCATCGACTTCAAGGAGGACGGCAACATCCTGGGGCACAA
GCTGGAGTACAACTACAACAGCCACAACGTCTATATCATGGCCGACAAGCAGAAGAACGGCATCAAGGTGAACTTCA
AGATCCGCCACAACATCGAGGACGGCAGCGTGCAGCTCGCCGACCACTACCAGCAGAACACCCCCATCGGCGACGGC
CCCGTGCTGCTGCCCGACAACCACTACCTGAGCTACCAGTCCAAACTGAGCAAAGACCCCAACGAGAAGCGCGATCA
CATGGTCCTGCTGGAGTTCGTGACCGCCGCCGGGATCACTCTCGGCATGGACGAGCTGTACAAGTGATGAATTgcGG
CCGCCTGACGTAACTAGTgTAAggcatgcaagcttgatatcaagcttatcgataatcaacctctggattacaaaatt
tgtgaaagattgactggtattcttaactatgttgctccttttacgctatgtggatacgctgctttaatgcctttgta
tcatgctattgcttcccgtatggctttcattttctcctccttgtataaatcctggttgctgtctctttatgaggagt
tgtggcccgttgtcaggcaacgtggcgtggtgtgcactgtgtttgctgacgcaacccccactggttggggcattgcc
accacctgtcagctcctttccgggactttcgctttccccctccctattgccacggcggaactcatcgccgcctgcct
tgcccgctgctggacaggggctcggctgttgggcactgacaattccgtggtgttgtcggggaaatcatcgtcctttc
cttggctgctcgcctgtgttgccacctggattctgcgcgggacgtccttctgctacgtcccttcggccctcaatcca
gcggaccttccttcccgcggcctgctgccggctctgcggcctcttccgcgtcttcgccttcgccctcagacgagtcg
gatctccctttgggccgcctccccgcat

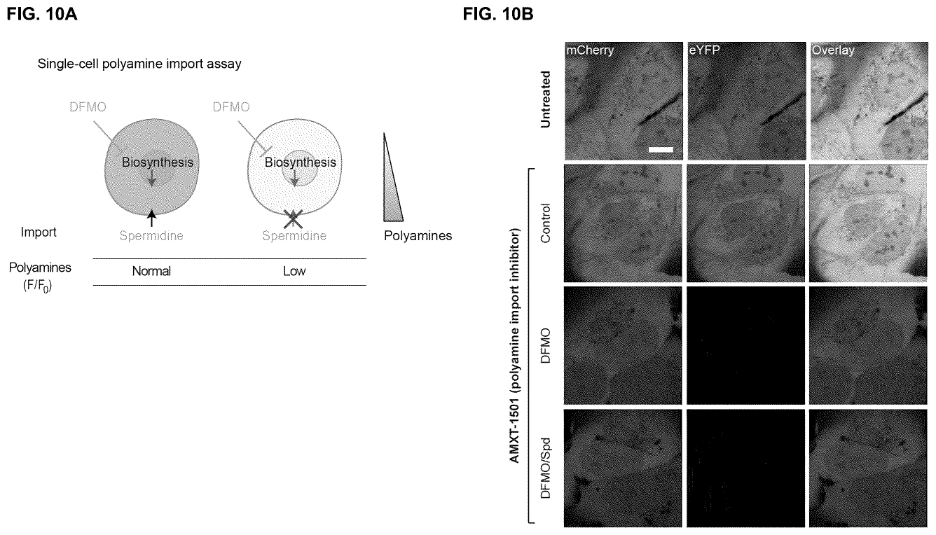

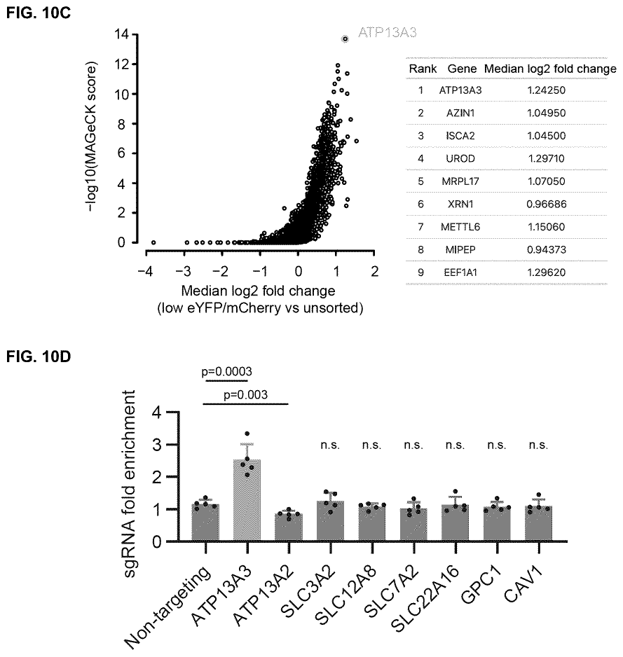

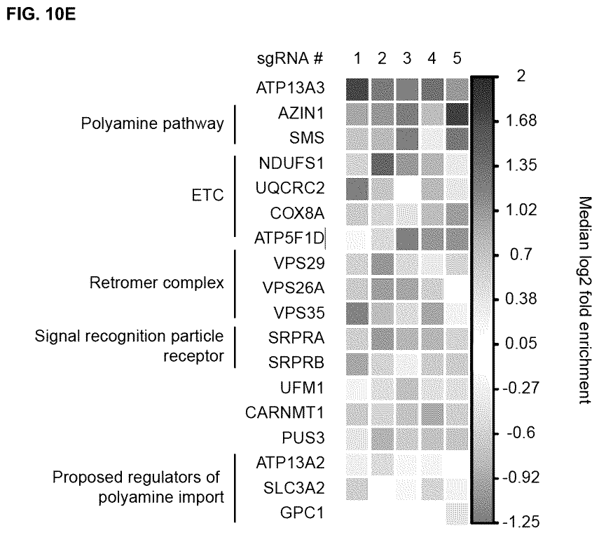

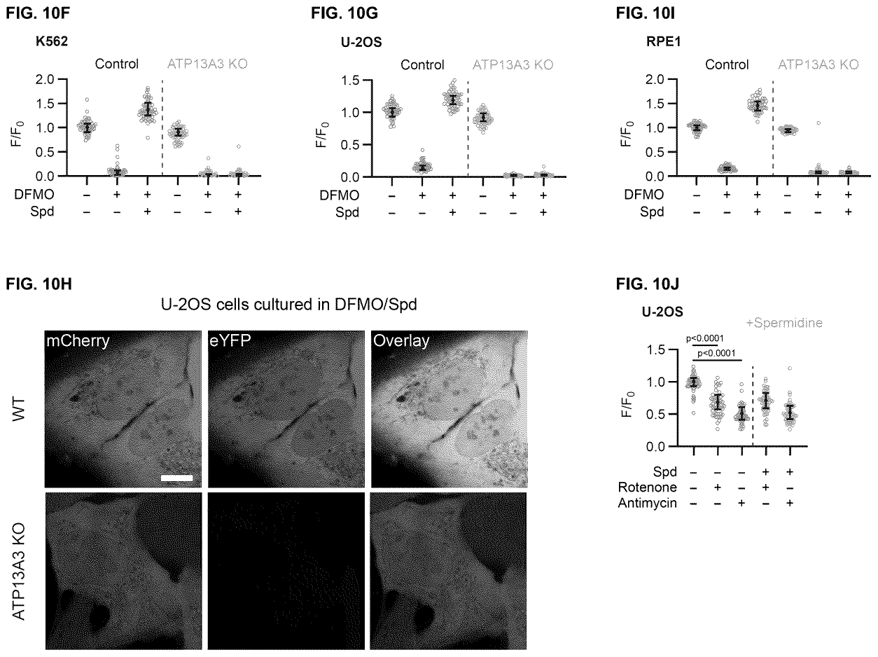

ATP13A3 KO Vector (pLenti-hU6-ATP13A3_sgRNA EF1-SpCas9-FLAG-P2A-Puro-WPRE, See FIGS. 10F-10I)

(SEQ ID NO: 25)
gtcgacggatcgggagatctcccgatcccctatggtgcactctcagtacaatctgctctgatgccgcatagttaagc
cagtatctgctccctgcttgtgtgttggaggtcgctgagtagtgcgcgagcaaaatttaagctacaacaaggcaagg
cttgaccgacaattgcatgaagaatctgcttagggttaggcgttttgcgctgcttcgcgatgtacgggccagatata
cgcgttgacattgattattgactagttattaatagtaatcaattacggggtcattagttcatagcccatatatggag
ttccgcgttacataacttacggtaaatggcccgcctggctgaccgcccaacgacccccgcccattgacgtcaataat
gacgtatgttcccatagtaacgccaatagggactttccattgacgtcaatgggtggagtatttacggtaaactgccc
acttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccat
ggtgatgcggttttggcagtacatcaatgggcgtggatagcggtttgactcacggggatttccaagtctccacccca
ttgacgtcaatgggagtttgttttggcaccaaaatcaacgggactttccaaaatgtcgtaacaactccgccccattg
acgcaaatgggcggtaggcgtgtacggtgggaggtctatataagcagcgcgttttgcctgtactgggtctctctggt
tagaccagatctgagcctgggagctctctggctaactagggaacccactgcttaagcctcaataaagcttgccttga
gtgcttcaagtagtgtgtgcccgtctgttgtgtgactctggtaactagagatccctcagacccttttagtcagtgtg
gaaaatctctagcagtggcgcccgaacagggacttgaaagcgaaagggaaaccagaggagctctctcgacgcaggac
tcggcttgctgaagcgcgcacggcaagaggcgaggggcggcgactggtgagtacgccaaaaattttgactagcggag
gctagaaggagagagatgggtgcgagagcgtcagtattaagcgggggagaattagatcgcgatgggaaaaaattcgg
ttaaggccagggggaaagaaaaaatataaattaaaacatatagtatgggcaagcagggagctagaacgattcgcagt
taatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacacc
aaggaagctttagacaagatagaggaagagcaaaacaaaagtaagaccaccgcacagcaagcggccgctgatcttca
gacctggaggaggagatatgagggacaattggagaagtgaattatataaatataaagtagtaaaaattgaaccatta
ggagtagcacccaccaaggcaaagagaagagtggtgcagagagaaaaaagagcagtgggaataggagctttgttcct
tgggttcttgggagcagcaggaagcactatgggcgcagcgtcaatgacgctgacggtacaggccagacaattattgt
ctggtatagtgcagcagcagaacaatttgctgagggctattgaggcgcaacagcatctgttgcaactcacagtctgg
ggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagattt
ggaatcacacgacctggatggagtgggacagagaaattaacaattacacaagcttaatacactccttaattgaagaa
tcgcaaaaccagcaagaaaagaatgaacaagaattattggaattagataaatgggcaagtttgtggaattggtttaa
cataacaaattggctgtggtatataaaattattcataatgatagtaggaggcttggtaggtttaagaatagtttttg
ctgtactttctatagtgaatagagttaggcagggatattcaccattatcgtttcagacccacctcccaaccccgagg
ggacccgacaggcccgaaggaatagaagaagaaggtggagagagagacagagacagatccattcgattagtgaacgg
atcggcactgcgtgcgccaattctgcagacaaatggcagtattcatccacaattttaaaagaaaaggggggattggg
gggtacagtgcaggggaaagaatagtagacataatagcaacagacatacaaactaaagaattacaaaaacaaattac
aaaaattcaaaattttcgggtttattacagggacagcagagatccagtttggttaattaaggtaccgagggcctatt
tcccatgattccttcatatttgcatatacgatacaaggctgttagagagataattagaattaatttgactgtaaaca
caaagatattagtacaaaatacgtgacgtagaaagtaataatttcttgggtagtttgcagttttaaaattatgtttt
aaaatggactatcatatgcttaccgtaacttgaaagtatttcgatttcttggctttatatatcttGTGGAAAGGACG
AAAcaccgTGGGTGAAATAACGAATCTGGTTTaAGAGCTAtgctgGAAAcagcaTAGCAAGTTtAAATAAGGCTAGT
CCGTTATCAACTTGAAAAAGTGGCACCGAGTCGGTGCTTTTTTgaattcgctagctaggtcttgaaaggagtgggaa
ttggctccggtgcccgtcagtgggcagagcgcacatcgcccacagtccccgagaagttggggggaggggtcggcaat
tgatccggtgcctagagaaggtggcgcggggtaaactgggaaagtgatgtcgtgtactggctccgcctttttcccga
gggtgggggagaaccgtatataagtgcagtagtcgccgtgaacgttctttttcgcaacgggtttgccgccagaacac
aggaccggttctagagcgctgccaccATGGACAAGAAGTACAGCATCGGCCTGGACATCGGCACCAACTCTGTGGGC
TGGGCCGTGATCACCGACGAGTACAAGGTGCCCAGCAAGAAATTCAAGGTGCTGGGCAACACCGACCGGCACAGCAT
CAAGAAGAACCTGATCGGAGCCCTGCTGTTCGACAGCGGCGAAACAGCCGAGGCCACCCGGCTGAAGAGAACCGCCA
GAAGAAGATACACCAGACGGAAGAACCGGATCTGCTATCTGCAAGAGATCTTCAGCAACGAGATGGCCAAGGTGGAC
GACAGCTTCTTCCACAGACTGGAAGAGTCCTTCCTGGTGGAAGAGGATAAGAAGCACGAGCGGCACCCCATCTTCGG
CAACATCGTGGACGAGGTGGCCTACCACGAGAAGTACCCCACCATCTACCACCTGAGAAAGAAACTGGTGGACAGCA
CCGACAAGGCCGACCTGCGGCTGATCTATCTGGCCCTGGCCCACATGATCAAGTTCCGGGGCCACTTCCTGATCGAG
GGCGACCTGAACCCCGACAACAGCGACGTGGACAAGCTGTTCATCCAGCTGGTGCAGACCTACAACCAGCTGTTCGA
GGAAAACCCCATCAACGCCAGCGGCGTGGACGCCAAGGCCATCCTGTCTGCCAGACTGAGCAAGAGCAGACGGCTGG
AAAATCTGATCGCCCAGCTGCCCGGCGAGAAGAAGAATGGCCTGTTCGGAAACCTGATTGCCCTGAGCCTGGGCCTG
ACCCCCAACTTCAAGAGCAACTTCGACCTGGCCGAGGATGCCAAACTGCAGCTGAGCAAGGACACCTACGACGACGA
CCTGGACAACCTGCTGGCCCAGATCGGCGACCAGTACGCCGACCTGTTTCTGGCCGCCAAGAACCTGTCCGACGCCA
TCCTGCTGAGCGACATCCTGAGAGTGAACACCGAGATCACCAAGGCCCCCCTGAGCGCCTCTATGATCAAGAGATAC
GACGAGCACCACCAGGACCTGACCCTGCTGAAAGCTCTCGTGCGGCAGCAGCTGCCTGAGAAGTACAAAGAGATTTT
CTTCGACCAGAGCAAGAACGGCTACGCCGGCTACATTGACGGCGGAGCCAGCCAGGAAGAGTTCTACAAGTTCATCA
AGCCCATCCTGGAAAAGATGGACGGCACCGAGGAACTGCTCGTGAAGCTGAACAGAGAGGACCTGCTGCGGAAGCAG
CGGACCTTCGACAACGGCAGCATCCCCCACCAGATCCACCTGGGAGAGCTGCACGCCATTCTGCGGCGGCAGGAAGA
TTTTTACCCATTCCTGAAGGACAACCGGGAAAAGATCGAGAAGATCCTGACCTTCCGCATCCCCTACTACGTGGGCC
CTCTGGCCAGGGGAAACAGCAGATTCGCCTGGATGACCAGAAAGAGCGAGGAAACCATCACCCCCTGGAACTTCGAG
GAAGTGGTGGACAAGGGCGCTTCCGCCCAGAGCTTCATCGAGCGGATGACCAACTTCGATAAGAACCTGCCCAACGA
GAAGGTGCTGCCCAAGCACAGCCTGCTGTACGAGTACTTCACCGTGTATAACGAGCTGACCAAAGTGAAATACGTGA
CCGAGGGAATGAGAAAGCCCGCCTTCCTGAGCGGCGAGCAGAAAAAGGCCATCGTGGACCTGCTGTTCAAGACCAAC
CGGAAAGTGACCGTGAAGCAGCTGAAAGAGGACTACTTCAAGAAAATCGAGTGCTTCGACTCCGTGGAAATCTCCGG
CGTGGAAGATCGGTTCAACGCCTCCCTGGGCACATACCACGATCTGCTGAAAATTATCAAGGACAAGGACTTCCTGG
ACAATGAGGAAAACGAGGACATTCTGGAAGATATCGTGCTGACCCTGACACTGTTTGAGGACAGAGAGATGATCGAG
GAACGGCTGAAAACCTATGCCCACCTGTTCGACGACAAAGTGATGAAGCAGCTGAAGCGGCGGAGATACACCGGCTG
GGGCAGGCTGAGCCGGAAGCTGATCAACGGCATCCGGGACAAGCAGTCCGGCAAGACAATCCTGGATTTCCTGAAGT
CCGACGGCTTCGCCAACAGAAACTTCATGCAGCTGATCCACGACGACAGCCTGACCTTTAAAGAGGACATCCAGAAA
GCCCAGGTGTCCGGCCAGGGCGATAGCCTGCACGAGCACATTGCCAATCTGGCCGGCAGCCCCGCCATTAAGAAGGG
CATCCTGCAGACAGTGAAGGTGGTGGACGAGCTCGTGAAAGTGATGGGCCGGCACAAGCCCGAGAACATCGTGATCG
AAATGGCCAGAGAGAACCAGACCACCCAGAAGGGACAGAAGAACAGCCGCGAGAGAATGAAGCGGATCGAAGAGGGC
ATCAAAGAGCTGGGCAGCCAGATCCTGAAAGAACACCCCGTGGAAAACACCCAGCTGCAGAACGAGAAGCTGTACCT
GTACTACCTGCAGAATGGGCGGGATATGTACGTGGACCAGGAACTGGACATCAACCGGCTGTCCGACTACGATGTGG
ACCATATCGTGCCTCAGAGCTTTCTGAAGGACGACTCCATCGACAACAAGGTGCTGACCAGAAGCGACAAGAACCGG
GGCAAGAGCGACAACGTGCCCTCCGAAGAGGTCGTGAAGAAGATGAAGAACTACTGGCGGCAGCTGCTGAACGCCAA
GCTGATTACCCAGAGAAAGTTCGACAATCTGACCAAGGCCGAGAGAGGCGGCCTGAGCGAACTGGATAAGGCCGGCT
TCATCAAGAGACAGCTGGTGGAAACCCGGCAGATCACAAAGCACGTGGCACAGATCCTGGACTCCCGGATGAACACT
AAGTACGACGAGAATGACAAGCTGATCCGGGAAGTGAAAGTGATCACCCTGAAGTCCAAGCTGGTGTCCGATTTCCG
GAAGGATTTCCAGTTTTACAAAGTGCGCGAGATCAACAACTACCACCACGCCCACGACGCCTACCTGAACGCCGTCG
TGGGAACCGCCCTGATCAAAAAGTACCCTAAGCTGGAAAGCGAGTTCGTGTACGGCGACTACAAGGTGTACGACGTG
CGGAAGATGATCGCCAAGAGCGAGCAGGAAATCGGCAAGGCTACCGCCAAGTACTTCTTCTACAGCAACATCATGAA
CTTTTTCAAGACCGAGATTACCCTGGCCAACGGCGAGATCCGGAAGCGGCCTCTGATCGAGACAAACGGCGAAACCG
GGGAGATCGTGTGGGATAAGGGCCGGGATTTTGCCACCGTGCGGAAAGTGCTGAGCATGCCCCAAGTGAATATCGTG
AAAAAGACCGAGGTGCAGACAGGCGGCTTCAGCAAAGAGTCTATCCTGCCCAAGAGGAACAGCGATAAGCTGATCGC
CAGAAAGAAGGACTGGGACCCTAAGAAGTACGGCGGCTTCGACAGCCCCACCGTGGCCTATTCTGTGCTGGTGGTGG
CCAAAGTGGAAAAGGGCAAGTCCAAGAAACTGAAGAGTGTGAAAGAGCTGCTGGGGATCACCATCATGGAAAGAAGC
AGCTTCGAGAAGAATCCCATCGACTTTCTGGAAGCCAAGGGCTACAAAGAAGTGAAAAAGGACCTGATCATCAAGCT
GCCTAAGTACTCCCTGTTCGAGCTGGAAAACGGCCGGAAGAGAATGCTGGCCTCTGCCGGCGAACTGCAGAAGGGAA
ACGAACTGGCCCTGCCCTCCAAATATGTGAACTTCCTGTACCTGGCCAGCCACTATGAGAAGCTGAAGGGCTCCCCC
GAGGATAATGAGCAGAAACAGCTGTTTGTGGAACAGCACAAGCACTACCTGGACGAGATCATCGAGCAGATCAGCGA
GTTCTCCAAGAGAGTGATCCTGGCCGACGCTAATCTGGACAAAGTGCTGTCCGCCTACAACAAGCACCGGGATAAGC
CCATCAGAGAGCAGGCCGAGAATATCATCCACCTGTTTACCCTGACCAATCTGGGAGCCCCTGCCGCCTTCAAGTAC
TTTGACACCACCATCGACCGGAAGAGGTACACCAGCACCAAAGAGGTGCTGGACGCCACCCTGATCCACCAGAGCAT
CACCGGCCTGTACGAGACACGGATCGACCTGTCTCAGCTGGGAGGCGACAAGCGACCTGCCGCCACAAAGAAGGCTG
GACAGGCTAAGAAGAAGAAAGATTACAAAGACGATGACGATAAGGGATCCGGCGCAACAAACTTCTCTCTGCTGAAA
CAAGCCGGAGATGTCGAAGAGAATCCTGGACCGACCGAGTACAAGCCCACGGTGCGCCTCGCCACCCGCGACGACGT
CCCCAGGGCCGTACGCACCCTCGCCGCCGCGTTCGCCGACTACCCCGCCACGCGCCACACCGTCGATCCGGACCGCC
ACATCGAGCGGGTCACCGAGCTGCAAGAACTCTTCCTCACGCGCGTCGGGCTCGACATCGGCAAGGTGTGGGTCGCG
GACGACGGCGCCGCGGTGGCGGTCTGGACCACGCCGGAGAGCGTCGAAGCGGGGGCGGTGTTCGCCGAGATCGGCCC
GCGCATGGCCGAGTTGAGCGGTTCCCGGCTGGCCGCGCAGCAACAGATGGAAGGCCTCCTGGCGCCGCACCGGCCCA
AGGAGCCCGCGTGGTTCCTGGCCACCGTCGGAGTCTCGCCCGACCACCAGGGCAAGGGTCTGGGCAGCGCCGTCGTG
CTCCCCGGAGTGGAGGCGGCCGAGCGCGCCGGGGTGCCCGCCTTCCTGGAGACCTCCGCGCCCCGCAACCTCCCCTT
CTACGAGCGGCTCGGCTTCACCGTCACCGCCGACGTCGAGGTGCCCGAAGGACCGCGCACCTGGTGCATGACCCGCA
AGCCCGGTGCCTGAACGCGTTAAGTCGACAATCAACCTCTGGATTACAAAATTTGTGAAAGATTGACTGGTATTCTT
AACTATGTTGCTCCTTTTACGCTATGTGGATACGCTGCTTTAATGCCTTTGTATCATGCTATTGCTTCCCGTATGGC
TTTCATTTTCTCCTCCTTGTATAAATCCTGGTTGCTGTCTCTTTATGAGGAGTTGTGGCCCGTTGTCAGGCAACGTG
GCGTGGTGTGCACTGTGTTTGCTGACGCAACCCCCACTGGTTGGGGCATTGCCACCACCTGTCAGCTCCTTTCCGGG
ACTTTCGCTTTCCCCCTCCCTATTGCCACGGCGGAACTCATCGCCGCCTGCCTTGCCCGCTGCTGGACAGGGGCTCG
GCTGTTGGGCACTGACAATTCCGTGGTGTTGTCGGGGAAATCATCGTCCTTTCCTTGGCTGCTCGCCTGTGTTGCCA
CCTGGATTCTGCGCGGGACGTCCTTCTGCTACGTCCCTTCGGCCCTCAATCCAGCGGACCTTCCTTCCCGCGGCCTG
CTGCCGGCTCTGCGGCCTCTTCCGCGTCTTCGCCTTCGCCCTCAGACGAGTCGGATCTCCCTTTGGGCCGCCTCCCC
GCGTCGACTTTAAGACCAATGACTTACAAGGCAGCTGTAGATCTTAGCCACTTTTTAAAAGAAAAGGGGGGACTGGA
AGGGCTAATTCACTCCCAACGAAGACAAGATCTGCTTTTTGCTTGTACTGGGTCTCTCTGGTTAGACCAGATCTGAG
CCTGGGAGCTCTCTGGCTAACTAGGGAACCCACTGCTTAAGCCTCAATAAAGCTTGCCTTGAGTGCTTCAAGTAGTG
TGTGCCCGTCTGTTGTGTGACTCTGGTAACTAGAGATCCCTCAGACCCTTTTAGTCAGTGTGGAAAATCTCTAGCAG
ggcccgtttaaacccgctgatcagcctcgactgtgccttctagttgccagccatctgttgtttgcccctcccccgtg
ccttccttgaccctggaaggtgccactcccactgtcctttcctaataaaatgaggaaattgcatcgcattgtctgag
taggtgtcattctattctggggggtggggtggggcaggacagcaagggggaggattgggaagacaatagcaggcatg
ctggggatgcggtgggctctatggcttctgaggcggaaagaaccagctggggctctagggggtatccccacgcgccc
tgtagcggcgcattaagcgcggcgggtgtggtggttacgcgcagcgtgaccgctacacttgccagcgccctagcgcc
cgctcctttcgctttcttcccttcctttctcgccacgttcgccggctttccccgtcaagctctaaatcgggggctcc
ctttagggttccgatttagtgctttacggcacctcgaccccaaaaaacttgattagggtgatggttcacgtagtggg
ccatcgccctgatagacggtttttcgccctttgacgttggagtccacgttctttaatagtggactcttgttccaaac
tggaacaacactcaaccctatctcggtctattcttttgatttataagggattttgccgatttcggcctattggttaa
aaaatgagctgatttaacaaaaatttaacgcgaattaattctgtggaatgtgtgtcagttagggtgtggaaagtccc
caggctccccagcaggcagaagtatgcaaagcatgcatctcaattagtcagcaaccaggtgtggaaagtccccaggc
tccccagcaggcagaagtatgcaaagcatgcatctcaattagtcagcaaccatagtcccgcccctaactccgcccat
cccgcccctaactccgcccagttccgcccattctccgccccatggctgactaattttttttatttatgcagaggccg
aggccgcctctgcctctgagctattccagaagtagtgaggaggcttttttggaggcctaggcttttgcaaaaagctc
ccgggagcttgtatatccattttcggatctgatcagcacgtgttgacaattaatcatcggcatagtatatcggcata
gtataatacgacaaggtgaggaactaaaccatggccaagttgaccagtgccgttccggtgctcaccgcgcgcgacgt
cgccggagcggtcgagttctggaccgaccggctcgggttctcccgggacttcgtggaggacgacttcgccggtgtgg
tccgggacgacgtgaccctgttcatcagcgcggtccaggaccaggtggtgccggacaacaccctggcctgggtgtgg
gtgcgcggcctggacgagctgtacgccgagtggtcggaggtcgtgtccacgaacttccgggacgcctccgggccggc
catgaccgagatcggcgagcagccgtgggggcgggagttcgccctgcgcgacccggccggcaactgcgtgcacttcg
tggccgaggagcaggactgacacgtgctacgagatttcgattccaccgccgccttctatgaaaggttgggcttcgga
atcgttttccgggacgccggctggatgatcctccagcgcggggatctcatgctggagttcttcgcccaccccaactt
gtttattgcagcttataatggttacaaataaagcaatagcatcacaaatttcacaaataaagcatttttttcactgc
attctagttgtggtttgtccaaactcatcaatgtatcttatcatgtctgtataccgtcgacctctagctagagcttg
gcgtaatcatggtcatagctgtttcctgtgtgaaattgttatccgctcacaattccacacaacatacgagccggaag
cataaagtgtaaagcctggggtgcctaatgagtgagctaactcacattaattgcgttgcgctcactgcccgctttcc
agtcgggaaacctgtcgtgccagctgcattaatgaatcggccaacgcgcggggagaggcggtttgcgtattgggcgc
tcttccgcttcctcgctcactgactcgctgcgctcggtcgttcggctgcggcgagcggtatcagctcactcaaaggc
ggtaatacggttatccacagaatcaggggataacgcaggaaagaacatgtgagcaaaaggccagcaaaaggccagga
accgtaaaaaggccgcgttgctggcgtttttccataggctccgcccccctgacgagcatcacaaaaatcgacgctca
agtcagaggtggcgaaacccgacaggactataaagataccaggcgtttccccctggaagctccctcgtgcgctctcc
tgttccgaccctgccgcttaccggatacctgtccgcctttctcccttcgggaagcgtggcgctttctcatagctcac
gctgtaggtatctcagttcggtgtaggtcgttcgctccaagctgggctgtgtgcacgaaccccccgttcagcccgac
cgctgcgccttatccggtaactatcgtcttgagtccaacccggtaagacacgacttatcgccactggcagcagccac
tggtaacaggattagcagagcgaggtatgtaggcggtgctacagagttcttgaagtggtggcctaactacggctaca
ctagaagaacagtatttggtatctgcgctctgctgaagccagttaccttcggaaaaagagttggtagctcttgatcc
ggcaaacaaaccaccgctggtagcggtggtttttttgtttgcaagcagcagattacgcgcagaaaaaaaggatctca
agaagatcctttgatcttttctacggggtctgacgctcagtggaacgaaaactcacgttaagggattttggtcatga
gattatcaaaaaggatcttcacctagatccttttaaattaaaaatgaagttttaaatcaatctaaagtatatatgag
taaacttggtctgacagttaccaatgcttaatcagtgaggcacctatctcagcgatctgtctatttcgttcatccat
agttgcctgactccccgtcgtgtagataactacgatacgggagggcttaccatctggccccagtgctgcaatgatac
cgcgagacccacgctcaccggctccagatttatcagcaataaaccagccagccggaagggccgagcgcagaagtggt
cctgcaactttatccgcctccatccagtctattaattgttgccgggaagctagagtaagtagttcgccagttaatag
tttgcgcaacgttgttgccattgctacaggcatcgtggtgtcacgctcgtcgtttggtatggcttcattcagctccg
gttcccaacgatcaaggcgagttacatgatcccccatgttgtgcaaaaaagcggttagctccttcggtcctccgatc
gttgtcagaagtaagttggccgcagtgttatcactcatggttatggcagcactgcataattctcttactgtcatgcc
atccgtaagatgcttttctgtgactggtgagtactcaaccaagtcattctgagaatagtgtatgcggcgaccgagtt
gctcttgcccggcgtcaatacgggataataccgcgccacatagcagaactttaaaagtgctcatcattggaaaacgt
tcttcggggcgaaaactctcaaggatcttaccgctgttgagatccagttcgatgtaacccactcgtgcacccaactg
atcttcagcatcttttactttcaccagcgtttctgggtgagcaaaaacaggaaggcaaaatgccgcaaaaaagggaa
taagggcgacacggaaatgttgaatactcatactcttcctttttcaatattattgaagcatttatcagggttattgt
ctcatgagcggatacatatttgaatgtatttagaaaaataaacaaataggggttccgcgcacatttccccgaaaagt
gccacctgac

AAVS1 Control (pLenti-hU6-AAVS1_sgRNA EF1-SpCas9-FLAG-P2A-Puro-WPRE, See FIGS. 10F-10I)

(SEQ ID NO: 26)
gtcgacggatcgggagatctcccgatcccctatggtgcactctcagtacaatctgctctgatgccgcatagttaagc
cagtatctgctccctgcttgtgtgttggaggtcgctgagtagtgcgcgagcaaaatttaagctacaacaaggcaagg
cttgaccgacaattgcatgaagaatctgcttagggttaggcgttttgcgctgcttcgcgatgtacgggccagatata
cgcgttgacattgattattgactagttattaatagtaatcaattacggggtcattagttcatagcccatatatggag
ttccgcgttacataacttacggtaaatggcccgcctggctgaccgcccaacgacccccgcccattgacgtcaataat
gacgtatgttcccatagtaacgccaatagggactttccattgacgtcaatgggtggagtatttacggtaaactgccc
acttggcagtacatcaagtgtatcatatgccaagtacgccccctattgacgtcaatgacggtaaatggcccgcctgg
cattatgcccagtacatgaccttatgggactttcctacttggcagtacatctacgtattagtcatcgctattaccat
ggtgatgcggttttggcagtacatcaatgggcgtggatagcggtttgactcacggggatttccaagtctccacccca
ttgacgtcaatgggagtttgttttggcaccaaaatcaacgggactttccaaaatgtcgtaacaactccgccccattg
acgcaaatgggcggtaggcgtgtacggtgggaggtctatataagcagcgcgttttgcctgtactgggtctctctggt
tagaccagatctgagcctgggagctctctggctaactagggaacccactgcttaagcctcaataaagcttgccttga
gtgcttcaagtagtgtgtgcccgtctgttgtgtgactctggtaactagagatccctcagacccttttagtcagtgtg
gaaaatctctagcagtggcgcccgaacagggacttgaaagcgaaagggaaaccagaggagctctctcgacgcaggac
tcggcttgctgaagcgcgcacggcaagaggcgaggggcggcgactggtgagtacgccaaaaattttgactagcggag
gctagaaggagagagatgggtgcgagagcgtcagtattaagcgggggagaattagatcgcgatgggaaaaaattcgg
ttaaggccagggggaaagaaaaaatataaattaaaacatatagtatgggcaagcagggagctagaacgattcgcagt
taatcctggcctgttagaaacatcagaaggctgtagacaaatactgggacagctacaaccatcccttcagacaggat
cagaagaacttagatcattatataatacagtagcaaccctctattgtgtgcatcaaaggatagagataaaagacacc
aaggaagctttagacaagatagaggaagagcaaaacaaaagtaagaccaccgcacagcaagcggccgctgatcttca
gacctggaggaggagatatgagggacaattggagaagtgaattatataaatataaagtagtaaaaattgaaccatta
ggagtagcacccaccaaggcaaagagaagagtggtgcagagagaaaaaagagcagtgggaataggagctttgttcct
tgggttcttgggagcagcaggaagcactatgggcgcagcgtcaatgacgctgacggtacaggccagacaattattgt
ctggtatagtgcagcagcagaacaatttgctgagggctattgaggcgcaacagcatctgttgcaactcacagtctgg
ggcatcaagcagctccaggcaagaatcctggctgtggaaagatacctaaaggatcaacagctcctggggatttgggg
ttgctctggaaaactcatttgcaccactgctgtgccttggaatgctagttggagtaataaatctctggaacagattt
ggaatcacacgacctggatggagtgggacagagaaattaacaattacacaagcttaatacactccttaattgaagaa
tcgcaaaaccagcaagaaaagaatgaacaagaattattggaattagataaatgggcaagtttgtggaattggtttaa
cataacaaattggctgtggtatataaaattattcataatgatagtaggaggcttggtaggtttaagaatagtttttg
ctgtactttctatagtgaatagagttaggcagggatattcaccattatcgtttcagacccacctcccaaccccgagg
ggacccgacaggcccgaaggaatagaagaagaaggtggagagagagacagagacagatccattcgattagtgaacgg
atcggcactgcgtgcgccaattctgcagacaaatggcagtattcatccacaattttaaaagaaaaggggggattggg
gggtacagtgcaggggaaagaatagtagacataatagcaacagacatacaaactaaagaattacaaaaacaaattac
aaaaattcaaaattttcgggtttattacagggacagcagagatccagtttggttaattaaggtaccgagggcctatt
tcccatgattccttcatatttgcatatacgatacaaggctgttagagagataattagaattaatttgactgtaaaca
caaagatattagtacaaaatacgtgacgtagaaagtaataatttcttgggtagtttgcagttttaaaattatgtttt
aaaatggactatcatatgcttaccgtaacttgaaagtatttcgatttcttggctttatatatcttGTGGAAAGGACG
AAACACCgGGGCCACTAGGGACAGGATGTTTaAGAGCTAtgctgGAAAcagcaTAGCAAGTTtAAATAAGGCTAGTC
CGTTATCAACTTGAAAAAGTGGCACCGAGTCGGTGCTTTTTTgaattcgctagctaggtcttgaaaggagtgggaat
tggctccggtgcccgtcagtgggcagagcgcacatcgcccacagtccccgagaagttggggggaggggtcggcaatt
gatccggtgcctagagaaggtggcgcggggtaaactgggaaagtgatgtcgtgtactggctccgcctttttcccgag
ggtgggggagaaccgtatataagtgcagtagtcgccgtgaacgttctttttcgcaacgggtttgccgccagaacaca
ggaccggttctagagcgctgccaccATGGACAAGAAGTACAGCATCGGCCTGGACATCGGCACCAACTCTGTGGGCT
GGGCCGTGATCACCGACGAGTACAAGGTGCCCAGCAAGAAATTCAAGGTGCTGGGCAACACCGACCGGCACAGCATC
AAGAAGAACCTGATCGGAGCCCTGCTGTTCGACAGCGGCGAAACAGCCGAGGCCACCCGGCTGAAGAGAACCGCCAG
AAGAAGATACACCAGACGGAAGAACCGGATCTGCTATCTGCAAGAGATCTTCAGCAACGAGATGGCCAAGGTGGACG
ACAGCTTCTTCCACAGACTGGAAGAGTCCTTCCTGGTGGAAGAGGATAAGAAGCACGAGCGGCACCCCATCTTCGGC
AACATCGTGGACGAGGTGGCCTACCACGAGAAGTACCCCACCATCTACCACCTGAGAAAGAAACTGGTGGACAGCAC
CGACAAGGCCGACCTGCGGCTGATCTATCTGGCCCTGGCCCACATGATCAAGTTCCGGGGCCACTTCCTGATCGAGG
GCGACCTGAACCCCGACAACAGCGACGTGGACAAGCTGTTCATCCAGCTGGTGCAGACCTACAACCAGCTGTTCGAG
GAAAACCCCATCAACGCCAGCGGCGTGGACGCCAAGGCCATCCTGTCTGCCAGACTGAGCAAGAGCAGACGGCTGGA
AAATCTGATCGCCCAGCTGCCCGGCGAGAAGAAGAATGGCCTGTTCGGAAACCTGATTGCCCTGAGCCTGGGCCTGA
CCCCCAACTTCAAGAGCAACTTCGACCTGGCCGAGGATGCCAAACTGCAGCTGAGCAAGGACACCTACGACGACGAC
CTGGACAACCTGCTGGCCCAGATCGGCGACCAGTACGCCGACCTGTTTCTGGCCGCCAAGAACCTGTCCGACGCCAT
CCTGCTGAGCGACATCCTGAGAGTGAACACCGAGATCACCAAGGCCCCCCTGAGCGCCTCTATGATCAAGAGATACG
ACGAGCACCACCAGGACCTGACCCTGCTGAAAGCTCTCGTGCGGCAGCAGCTGCCTGAGAAGTACAAAGAGATTTTC
TTCGACCAGAGCAAGAACGGCTACGCCGGCTACATTGACGGCGGAGCCAGCCAGGAAGAGTTCTACAAGTTCATCAA
GCCCATCCTGGAAAAGATGGACGGCACCGAGGAACTGCTCGTGAAGCTGAACAGAGAGGACCTGCTGCGGAAGCAGC
GGACCTTCGACAACGGCAGCATCCCCCACCAGATCCACCTGGGAGAGCTGCACGCCATTCTGCGGCGGCAGGAAGAT
TTTTACCCATTCCTGAAGGACAACCGGGAAAAGATCGAGAAGATCCTGACCTTCCGCATCCCCTACTACGTGGGCCC
TCTGGCCAGGGGAAACAGCAGATTCGCCTGGATGACCAGAAAGAGCGAGGAAACCATCACCCCCTGGAACTTCGAGG
AAGTGGTGGACAAGGGCGCTTCCGCCCAGAGCTTCATCGAGCGGATGACCAACTTCGATAAGAACCTGCCCAACGAG
AAGGTGCTGCCCAAGCACAGCCTGCTGTACGAGTACTTCACCGTGTATAACGAGCTGACCAAAGTGAAATACGTGAC
CGAGGGAATGAGAAAGCCCGCCTTCCTGAGCGGCGAGCAGAAAAAGGCCATCGTGGACCTGCTGTTCAAGACCAACC
GGAAAGTGACCGTGAAGCAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAGGACAAGGACTTCCTGGA
CAATGAGGAAAACGAGGACATTCTGGAAGATATCGTGCTGACCCTGACACTGTTTGAGGACAGAGAGATGATCGAGG
AACGGCTGAAAACCTATGCCCACCTGTTCGACGACAAAGTGATGAAGCAGCTGAAGCGGCGGAGATACACCGGCTGG
GGCAGGCTGAGCCGGAAGCTGATCAACGGCATCCGGGACAAGCAGTCCGGCAAGACAATCCTGGATTTCCTGAAGTC
CGACGGCTTCGCCAACAGAAACTTCATGCAGCTGATCCACGACGACAGCCTGACCTTTAAAGAGGACATCCAGAAAG
CCCAGGTGTCCGGCCAGGGCGATAGCCTGCACGAGCACATTGCCAATCTGGCCGGCAGCCCCGCCATTAAGAAGGGC
ATCCTGCAGACAGTGAAGGTGGTGGACGAGCTCGTGAAAGTGATGGGCCGGCACAAGCCCGAGAACATCGTGATCGA
AATGGCCAGAGAGAACCAGACCACCCAGAAGGGACAGAAGAACAGCCGCGAGAGAATGAAGCGGATCGAAGAGGGCA
TCAAAGAGCTGGGCAGCCAGATCCTGAAAGAACACCCCGTGGAAAACACCCAGCTGCAGAACGAGAAGCTGTACCTG
TACTACCTGCAGAATGGGCGGGATATGTACGTGGACCAGGAACTGGACATCAACCGGCTGTCCGACTACGATGTGGA
CCATATCGTGCCTCAGAGCTTTCTGAAGGACGACTCCATCGACAACAAGGTGCTGACCAGAAGCGACAAGAACCGGG
GCAAGAGCGACAACGTGCCCTCCGAAGAGGTCGTGAAGAAGATGAAGAACTACTGGCGGCAGCTGCTGAACGCCAAG
CTGATTACCCAGAGAAAGTTCGACAATCTGACCAAGGCCGAGAGAGGCGGCCTGAGCGAACTGGATAAGGCCGGCTT
CATCAAGAGACAGCTGGTGGAAACCCGGCAGATCACAAAGCACGTGGCACAGATCCTGGACTCCCGGATGAACACTA
AGTACGACGAGAATGACAAGCTGATCCGGGAAGTGAAAGTGATCACCCTGAAGTCCAAGCTGGTGTCCGATTTCCGG
AAGGATTTCCAGTTTTACAAAGTGCGCGAGATCAACAACTACCACCACGCCCACGACGCCTACCTGAACGCCGTCGT
GGGAACCGCCCTGATCAAAAAGTACCCTAAGCTGGAAAGCGAGTTCGTGTACGGCGACTACAAGGTGTACGACGTGC
GGAAGATGATCGCCAAGAGCGAGCAGGAAATCGGCAAGGCTACCGCCAAGTACTTCTTCTACAGCAACATCATGAAC
TTTTTCAAGACCGAGATTACCCTGGCCAACGGCGAGATCCGGAAGCGGCCTCTGATCGAGACAAACGGCGAAACCGG
GGAGATCGTGTGGGATAAGGGCCGGGATTTTGCCACCGTGCGGAAAGTGCTGAGCATGCCCCAAGTGAATATCGTGA
AAAAGACCGAGGTGCAGACAGGCGGCTTCAGCAAAGAGTCTATCCTGCCCAAGAGGAACAGCGATAAGCTGATCGCC
AGAAAGAAGGACTGGGACCCTAAGAAGTACGGCGGCTTCGACAGCCCCACCGTGGCCTATTCTGTGCTGGTGGTGGC
CAAAGTGGAAAAGGGCAAGTCCAAGAAACTGAAGAGTGTGAAAGAGCTGCTGGGGATCACCATCATGGAAAGAAGCA
GCTTCGAGAAGAATCCCATCGACTTTCTGGAAGCCAAGGGCTACAAAGAAGTGAAAAAGGACCTGATCATCAAGCTG
CCTAAGTACTCCCTGTTCGAGCTGGAAAACGGCCGGAAGAGAATGCTGGCCTCTGCCGGCGAACTGCAGAAGGGAAA
CGAACTGGCCCTGCCCTCCAAATATGTGAACTTCCTGTACCTGGCCAGCCACTATGAGAAGCTGAAGGGCTCCCCCG
AGGATAATGAGCAGAAACAGCTGTTTGTGGAACAGCACAAGCACTACCTGGACGAGATCATCGAGCAGATCAGCGAG
TTCTCCAAGAGAGTGATCCTGGCCGACGCTAATCTGGACAAAGTGCTGTCCGCCTACAACAAGCACCGGGATAAGCC
CATCAGAGAGCAGGCCGAGAATATCATCCACCTGTTTACCCTGACCAATCTGGGAGCCCCTGCCGCCTTCAAGTACT
TTGACACCACCATCGACCGGAAGAGGTACACCAGCACCAAAGAGGTGCTGGACGCCACCCTGATCCACCAGAGCATC
ACCGGCCTGTACGAGACACGGATCGACCTGTCTCAGCTGGGAGGCGACAAGCGACCTGCCGCCACAAAGAAGGCTGG
ACAGGCTAAGAAGAAGAAAGATTACAAAGACGATGACGATAAGGGATCCGGCGCAACAAACTTCTCTCTGCTGAAAC
AAGCCGGAGATGTCGAAGAGAATCCTGGACCGACCGAGTACAAGCCCACGGTGCGCCTCGCCACCCGCGACGACGTC
CCCAGGGCCGTACGCACCCTCGCCGCCGCGTTCGCCGACTACCCCGCCACGCGCCACACCGTCGATCCGGACCGCCA
CATCGAGCGGGTCACCGAGCTGCAAGAACTCTTCCTCACGCGCGTCGGGCTCGACATCGGCAAGGTGTGGGTCGCGG
ACGACGGCGCCGCGGTGGCGGTCTGGACCACGCCGGAGAGCGTCGAAGCGGGGGCGGTGTTCGCCGAGATCGGCCCG
CGCATGGCCGAGTTGAGCGGTTCCCGGCTGGCCGCGCAGCAACAGATGGAAGGCCTCCTGGCGCCGCACCGGCCCAA
GGAGCCCGCGTGGTTCCTGGCCACCGTCGGAGTCTCGCCCGACCACCAGGGCAAGGGTCTGGGCAGCGCCGTCGTGC
TCCCCGGAGTGGAGGCGGCCGAGCGCGCCGGGGTGCCCGCCTTCCTGGAGACCTCCGCGCCCCGCAACCTCCCCTTC
TACGAGCGGCTCGGCTTCACCGTCACCGCCGACGTCGAGGTGCCCGAAGGACCGCGCACCTGGTGCATGACCCGCAA
GCCCGGTGCCTGAACGCGTTAAGTCGACAATCAACCTCTGGATTACAAAATTTGTGAAAGATTGACTGGTATTCTTA
ACTATGTTGCTCCTTTTACGCTATGTGGATACGCTGCTTTAATGCCTTTGTATCATGCTATTGCTTCCCGTATGGCT
TTCATTTTCTCCTCCTTGTATAAATCCTGGTTGCTGTCTCTTTATGAGGAGTTGTGGCCCGTTGTCAGGCAACGTGG
CGTGGTGTGCACTGTGTTTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTGCTGGACAGGGGCTCGG
CTGTTGGGCACTGACAATTCCGTGGTGTTGTCGGGGAAATCATCGTCCTTTCCTTGGCTGCTCGCCTGTGTTGCCAC
CTGGATTCTGCGCGGGACGTCCTTCTGCTACGTCCCTTCGGCCCTCAATCCAGCGGACCTTCCTTCCCGCGGCCTGC
TGCCGGCTCTGCGGCCTCTTCCGCGTCTTCGCCTTCGCCCTCAGACGAGTCGGATCTCCCTTTGGGCCGCCTCCCCG
CGTCGACTTTAAGACCAATGACTTACAAGGCAGCTGTAGATCTTAGCCACTTTTTAAAAGAAAAGGGGGGACTGGAA
GGGCTAATTCACTCCCAACGAAGACAAGATCTGCTTTTTGCTTGTACTGGGTCTCTCTGGTTAGACCAGATCTGAGC
CTGGGAGCTCTCTGGCTAACTAGGGAACCCACTGCTTAAGCCTCAATAAAGCTTGCCTTGAGTGCTTCAAGTAGTGT
GTGCCCGTCTGTTGTGTGACTCTGGTAACTAGAGATCCCTCAGACCCTTTTAGTCAGTGTGGAAAATCTCTAGCAGg
gcccgtttaaacccgctgatcagcctcgactgtgccttctagttgccagccatctgttgtttgcccctcccccgtgc
cttccttgaccctggaaggtgccactcccactgtcctttcctaataaaatgaggaaattgcatcgcattgtctgagt
aggtgtcattctattctggggggtggggtggggcaggacagcaagggggaggattgggaagacaatagcaggcatgc
tggggatgcggtgggctctatggcttctgaggcggaaagaaccagctggggctctagggggtatccccacgcgccct
gtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccc
tttagggttccgatttagtgctttacggcacctcgaccccaaaaaacttgattagggtgatggttcacgtagtgggc
catcgccctgatagacggtttttcgccctttgacgttggagtccacgttctttaatagtggactcttgttccaaact
ggaacaacactcaaccctatctcggtctattcttttgatttataagggattttgccgatttcggcctattggttaaa
aaatgagctgatttaacaaaaatttaacgcgaattaattctgtggaatgtgtgtcagttagggtgtggaaagtcccc
aggctccccagcaggcagaagtatgcaaagcatgcatctcaattagtcagcaaccaggtgtggaaagtccccaggct
ccccagcaggcagaagtatgcaaagcatgcatctcaattagtcagcaaccatagtcccgcccctaactccgcccatc
ccgcccctaactccgcccagttccgcccattctccgccccatggctgactaattttttttatttatgcagaggccga
ggccgcctctgcctctgagctattccagaagtagtgaggaggcttttttggaggcctaggcttttgcaaaaagctcc
cgggagcttgtatatccattttcggatctgatcagcacgtgttgacaattaatcatcggcatagtatatcggcatag
tataatacgacaaggtgaggaactaaaccatggccaagttgaccagtgccgttccggtgctcaccgcgcgcgacgtc
gccggagcggtcgagttctggaccgaccggctcgggttctcccgggacttcgtggaggacgacttcgccggtgtggt
ccgggacgacgtgaccctgttcatcagcgcggtccaggaccaggtggtgccggacaacaccctggcctgggtgtggg
tgcgcggcctggacgagctgtacgccgagtggtcggaggtcgtgtccacgaacttccgggacgcctccgggccggcc
atgaccgagatcggcgagcagccgtgggggcgggagttcgccctgcgcgacccggccggcaactgcgtgcacttcgt
ggccgaggagcaggactgacacgtgctacgagatttcgattccaccgccgccttctatgaaaggttgggcttcggaa
tcgttttccgggacgccggctggatgatcctccagcgcggggatctcatgctggagttcttcgcccaccccaacttg
tttattgcagcttataatggttacaaataaagcaatagcatcacaaatttcacaaataaagcatttttttcactgca
ttctagttgtggtttgtccaaactcatcaatgtatcttatcatgtctgtataccgtcgacctctagctagagcttgg
cgtaatcatggtcatagctgtttcctgtgtgaaattgttatccgctcacaattccacacaacatacgagccggaagc
ataaagtgtaaagcctggggtgcctaatgagtgagctaactcacattaattgcgttgcgctcactgcccgctttcca
gtcgggaaacctgtcgtgccagctgcattaatgaatcggccaacgcgcggggagaggcggtttgcgtattgggcgct
cttccgcttcctcgctcactgactcgctgcgctcggtcgttcggctgcggcgagcggtatcagctcactcaaaggcg
gtaatacggttatccacagaatcaggggataacgcaggaaagaacatgtgagcaaaaggccagcaaaaggccaggaa
ccgtaaaaaggccgcgttgctggcgtttttccataggctccgcccccctgacgagcatcacaaaaatcgacgctcaa
gtcagaggtggcgaaacccgacaggactataaagataccaggcgtttccccctggaagctccctcgtgcgctctcct
gttccgaccctgccgcttaccggatacctgtccgcctttctcccttcgggaagcgtggcgctttctcatagctcacg
ctgtaggtatctcagttcggtgtaggtcgttcgctccaagctgggctgtgtgcacgaaccccccgttcagcccgacc
gctgcgccttatccggtaactatcgtcttgagtccaacccggtaagacacgacttatcgccactggcagcagccact
ggtaacaggattagcagagcgaggtatgtaggcggtgctacagagttcttgaagtggtggcctaactacggctacac
tagaagaacagtatttggtatctgcgctctgctgaagccagttaccttcggaaaaagagttggtagctcttgatccg
gcaaacaaaccaccgctggtagcggtggtttttttgtttgcaagcagcagattacgcgcagaaaaaaaggatctcaa
gaagatcctttgatcttttctacggggtctgacgctcagtggaacgaaaactcacgttaagggattttggtcatgag
attatcaaaaaggatcttcacctagatccttttaaattaaaaatgaagttttaaatcaatctaaagtatatatgagt
aaacttggtctgacagttaccaatgcttaatcagtgaggcacctatctcagcgatctgtctatttcgttcatccata
gttgcctgactccccgtcgtgtagataactacgatacgggagggcttaccatctggccccagtgctgcaatgatacc
gcgagacccacgctcaccggctccagatttatcagcaataaaccagccagccggaagggccgagcgcagaagtggtc
ctgcaactttatccgcctccatccagtctattaattgttgccgggaagctagagtaagtagttcgccagttaatagt
ttgcgcaacgttgttgccattgctacaggcatcgtggtgtcacgctcgtcgtttggtatggcttcattcagctccgg
ttcccaacgatcaaggcgagttacatgatcccccatgttgtgcaaaaaagcggttagctccttcggtcctccgatcg
ttgtcagaagtaagttggccgcagtgttatcactcatggttatggcagcactgcataattctcttactgtcatgcca
tccgtaagatgcttttctgtgactggtgagtactcaaccaagtcattctgagaatagtgtatgcggcgaccgagttg
ctcttgcccggcgtcaatacgggataataccgcgccacatagcagaactttaaaagtgctcatcattggaaaacgtt
cttcggggcgaaaactctcaaggatcttaccgctgttgagatccagttcgatgtaacccactcgtgcacccaactga
tcttcagcatcttttactttcaccagcgtttctgggtgagcaaaaacaggaaggcaaaatgccgcaaaaaagggaat
aagggcgacacggaaatgttgaatactcatactcttcctttttcaatattattgaagcatttatcagggttattgtc
tcatgagcggatacatatttgaatgtatttagaaaaataaacaaataggggttccgcgcacatttccccgaaaagtg
ccacctgac

### EXAMPLES

Polyamines (putrescine, spermidine, and spermine) are essential metabolites found across all life kingdoms. They are crucial for numerous cellular processes, including growth, regeneration, differentiation, immunity, and aging (Pegg (2016), Puleston (2021), Madeo (2018), Hofer (2022)). Dysregulation of polyamine metabolism is associated with Parkinson's disease (van Veen (2020)), intellectual disability (Snyder & Robinson (1969), Ziegler (2022)), and cancer (Casero (2018), Holbert (2022)). The precise functions of polyamines in the cell remain unclear (Miller-Fleming (2015)). A major challenge in studying polyamine biology is the lack of technologies to quantitatively probe these small metabolites in living cells. Direct measurement of cellular polyamines is challenging because of their small size and charge (FIG. 2A). Current methods for polyamine measurement often rely on chromatography or mass spectrometry (van Veen (2020), Tate (2019)).

While these methods have provided valuable insights into our understanding of polyamine homeostasis, they also have severe critical limitations, inherent to the chromatographic strategy. First, these methods offer only a snapshot of polyamine content averaged across the sample. Information on cell-to-cell and temporal variability, which may be biologically meaningful, is lost. Second, they often require large biological sample quantities and have limited throughput, precluding large-scale genetic or pharmacological screens. Third, these methods require polyamine extraction from their native cellular context using organic solvents and extensive chemical derivatization (Minocha (1990)). These procedures introduce variability due to losses during extraction and labeling, making it challenging to tease apart subtle differences. The commonly used polyamine functionalization schemes to facilitate chromatographic retention are also light-sensitive, rendering them susceptible to degradation and thus obscuring downstream analyses (Seiler (1970)).

Disclosed herein is a genetically encoded polyamine reporter that enables quantification of polyamine levels with single-cell resolution. The reporter incorporates the endogenous polyamine-responsive frameshift motif found in the ornithine decarboxylase antizyme 1 (OAZ1) gene, which plays a critical role in regulating polyamine homeostasis. The OAZ1 protein binds to and promotes the degradation of ornithine decarboxylase (ODC1), a rate-limiting enzyme in the polyamine biosynthesis pathway (Fong (1976)). The OAZ1 mRNA contains a premature stop codon, and a programmed +1 ribosomal frameshift is required for the production of full-length, catalytically active OAZ1 protein (FIG. 1A). The efficiency of this frameshifting is dependent on the cellular polyamine levels, constituting an autoregulatory feedback mechanism to maintain polyamine homeostasis (Matsufuji (1995), Rom & Kahana (1994)). A quantitative and live-cell readout for intracellular polyamine levels was generated by leveraging a polyamine-responsive frameshift module from OAZ1 and relaying this frameshifting efficiency to a fluorescent reporter (FIG. 1A). This reporter enabled identification of modulators of polyamine import through a genome-wide screen. An unexpected interaction between polyamine import and the mitochondrial respiratory complex was revealed.

### Example 1. Materials and Methods

### Cloning and Plasmid Preparation

Complete plasmid sequences are provided in the section entitled “Sequences.” The sequence encoding the full-length OAZ1 was obtained as double-stranded DNA fragments (IDT) and its various regions were cloned between PacI and BamHI sites in a doxycycline-inducible lentiviral transfer plasmid (pHR-Tre3G-mCherry-MCS-eYFP). pHR-Tre3G-mCherry-MCS-eYFP vector is derived by Gibson cloning from pHR-Tre3G-29xGGGGCC-12xMS2, a gift from Ron Vale (Addgene, Watertown, MA, plasmid #99149). CRISPR-dCas9-mediated knockdown of Ornithine decarboxylase (ODC1) and spermidine synthase (SRM) was performed using the plasmid pLV hU6-sgRNA hUbC-dCas9-KRAB-T2a-Puro (Addgene plasmid #71236), a gift from Charles Gersbach. A single-guide RNA (sgRNA) with high on-target efficiency to target either ODC1 or SRM was designed as described previously (Horlbeck (2016)). The sgRNA sequences were as follows: ODC1 (GGGCGGCGGCGGCTACAGGA (SEQ ID NO:8)) and SRM (GGTAGCGCGAGCGCCGGTGG (SEQ ID NO:9)). CRISPR-Cas9 mediated knockout of ATP13A3 was performed using pLenti-EF1-SpCas9-FLAG-P2A-Puro-WPRE was used (a gift from Heather Keys) with sgRNA sequence TGGGTGAAATAACGAATCTG (SEQ ID NO:10). For the live tracking experiments in FIG. 1, the sequence encoding ecDHFR and miRFP670-2 were incorporated using Gibson into a constitutive SFFV-driven lentiviral transfer plasmid, pHR-tdMS2CP-YFP-WPRE (Addgene #99151), a gift from Ron Vale. A plasmid containing ecDHFR sequence was a gift from lain Cheeseman and miRFP670-2 was amplified from pTubulin-miRFP670-2 (Addgene #197238), a gift from Kiryl Piatkevich. All cloning and plasmid preparations were performed in Stbl3 Escherichia coli cells (ThermoFisher Scientific, Waltham, MA, C7373-03) grown at 30° C. All plasmids were verified by Oxford Nanopore sequencing (Plasmidsaurus, Eugene, OR, or Quintara Bioscience, Cambridge, MA).

### Materials

The following reagents were purchased from the following vendors.

Sigma-Aldrich (St. Louis, MO): perchloric acid (244252), 1,7-diamino heptane (D17408), acetone (270725), toluene (34866), L-proline (P0380), acetonitrile (34851), sodium carbonate, dansyl chloride (311155), spermidine trihydrochloride (S2501), DMSO (276855), and aminoguanidine hydrochloride (396494).

Cayman Chemicals (Ann Arbor, MI): DL-α-difluoromethylornithine (DFMO; 16889), rotenone (13995), antimycin A complex (34799), and ribavirin (16757).

MedChemExpress (Monmouth Junction, NJ): Sardomozide dihydrochloride (HY-13746B) and AMXT-1501 (HY-124617A).

### Preparation of Compounds and Inhibitors.

Fresh stocks were prepared for all the amine compounds from powder before the experiments. Polyamines and aminoguanidine stocks were prepared to a final concentration of 100 mg/mL in phosphate buffered saline (PBS) for each experiment. Sardomozide was dissolved in dimethyl sulfoxide (DMSO) to final concentrations of 10 mM. DFMO (55 mM) and ribavirin (100 mM) stocks were prepared in water. All stocks were filtered through a 0.45 μm filter upon preparation.

### Cell Culture

U-2OS, HEK 293T, K562, RPE1 (immortalized), and SH-SY5Y were purchased from the American Type Culture Collection (ATCC) and authenticated by short tandem repeat profiling. U-2OS, HEK 293T, and RPE1 were propagated in Dulbecco's Modified Eagle Medium (DMEM, ThermoFisher Scientific, 11965126). Suspension cultures of K562 cells were cultured in Roswell Park Memorial Institute medium (RPMI-1640, ThermoFisher Scientific, 11875093). SH-SY5Y cells were grown in Eagle's Minimal Essential Medium (EMEM) (ATCC 30-2003)/F12 (ThermoFisher Scientific, 11765054). All media were supplemented with penicillin/streptomycin/glutamine (ThermoFisher Scientific, 10378016) and 10% (v/v) tetracycline-free fetal bovine serum (FBS) (ThermoFisher Scientific, 26140079), except for SH-SY5Y cells which had additional FBS (15%). Cells were maintained at 37° C. with 5% CO₂ in a humidified incubator. Cells were tested negative for Mycoplasma contamination.

### Cell Line Transduction

Cell lines stably expressing rtTA transactivator protein (Takara Bio USA, Inc., Mountain View, CA) were generated via lentiviral infection. These stable cell lines were then transduced with the vector expressing OAZ1 shift site-containing polyamine reporter under a tetracycline-inducible promoter. Lentivirus was generated as follows. The cloned lentivirus transfer plasmids (2 μg), the packaging plasmid psPAX2 (1 μg), and the envelope plasmid pCMV-VSV-G (0.5 μg) were mixed with 8 μL of lipofectamine LTX (ThermoFisher Scientific, 15338-100) in 500 μL of Opti-Minimal Essential Medium (MEM) reduced serum media (ThermoFisher Scientific, 31985-070) for transfection of HEK293T cells according to the manufacturer's recommended protocol. After two days, the lentiviral particles were collected by passing the supernatant through a 0.45-μm filter and used to transduce cells supplemented with 10 μg/mL polybrene (MilliporeSigma, Burlington, MA, TR1003G). Polybrene was excluded during the transduction of SH-SY5Y cells due to toxicity. Transgene expression of the polyamine sensor was induced by adding 1,000 ng/mL doxycycline (Sigma-Aldrich) for 18 hours (hr) except where otherwise specified.

### Crypt Isolation and Culturing

As previously reported (Mihaylova (2018)) and briefly summarized here, small intestines were removed, washed with cold PBS, opened longitudinally, and then incubated on the shaker machine at 4° C. with PBS plus Ethylenediaminetetraacetic Acid (EDTA) (10 mM) for 45 minutes (min). Tissues were then moved to PBS. Crypts were then mechanically separated from the connective tissue by shaking and then filtered through a 70-μm mesh into a 50 mL conical tube to remove villus material and tissue fragments. Isolated crypts for cultures were embedded in Matrigel (Corning, Corning, NY, 356231, growth factor reduced) and cultured in a modified form of the medium as described previously (Sato (2009), Yilmaz (2012)). Crypt culture media consists of Advanced DMEM (ThermoFisher Scientific, 12491015) that was supplemented with epidermal growth factor (EGF) 40 ng/ml (ThermoFisher Scientific, 315-09), Noggin 200 ng/ml (ThermoFisher Scientific, 250-38), R-spondin 500 ng/ml (ThermoFisher Scientific, 315-32), B27 1X (ThermoFisher Scientific, 17504044), CHIR99021 3 μM (LC laboratories, Woburn, MA, C-6556), and Y-27632 dihydrochloride monohydrate 10 μM (Sigma-Aldrich, Y0503). Matrigel is allowed to solidify for 20-30 min in a 37° C. incubator. The culture medium was then overlaid onto the Matrigel, changed every three days, and maintained at 37° C. in fully humidified chambers containing 5% CO₂.

### Establishing Polyamine-Sensor Organoids

To generate the polyamine sensor-organoids, organoids derived from mouse intestines were dissociated by Triton-X at 37° C. for 2 min, mixed with virus supernatant (prepared as in cell lines transduction) and polybrene at 9 μg/ml, and then transferred to 48 well plates. The plate was centrifuged at 600×g at 30° C. for 1 hr and incubated in chambers for 3 hr. Collected samples were embedded in Matrigel and incubated with crypt medium plus 5% FBS for 7 days. 1,000 ng/mL of doxycycline was added into the medium and organoids were incubated for 24 hr to express the fluorescent protein, and then fluorescent-positive organoids were picked up under the fluorescent scope. For the DFMO and spermidine treatment, organoids were treated after the passaging. 1 mM DFMO with or without 50 μM spermidine was added to the culture medium, and the images of organoids were taken after 3 days of treatment. The commonly used organoid medium supplement B27 contains polyamines, and thus differences in the polyamine levels upon DFMO treatment become apparent only in its absence.

### Enzymatic Total Polyamine Assay

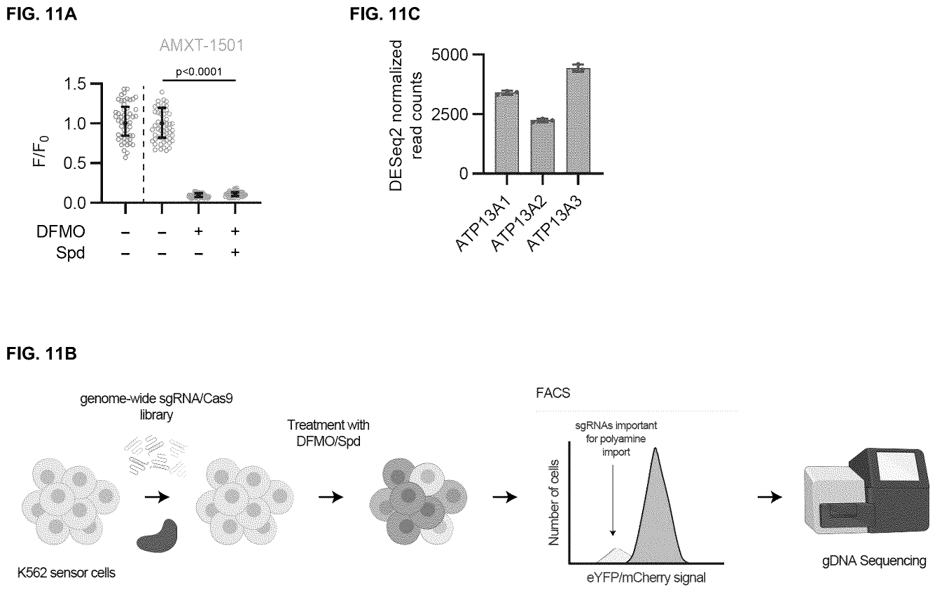

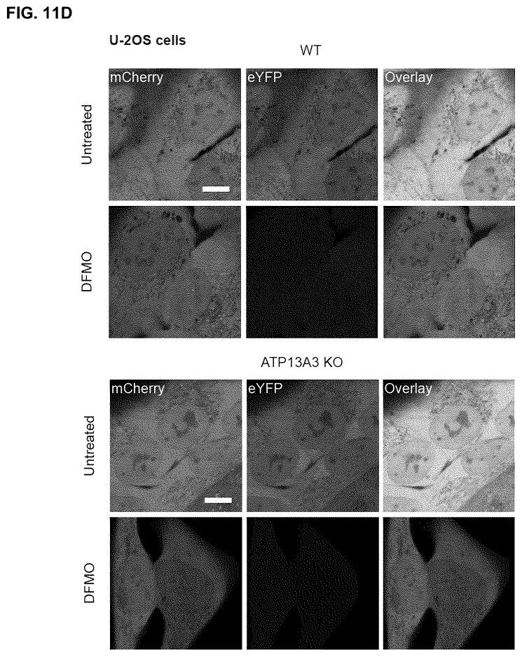

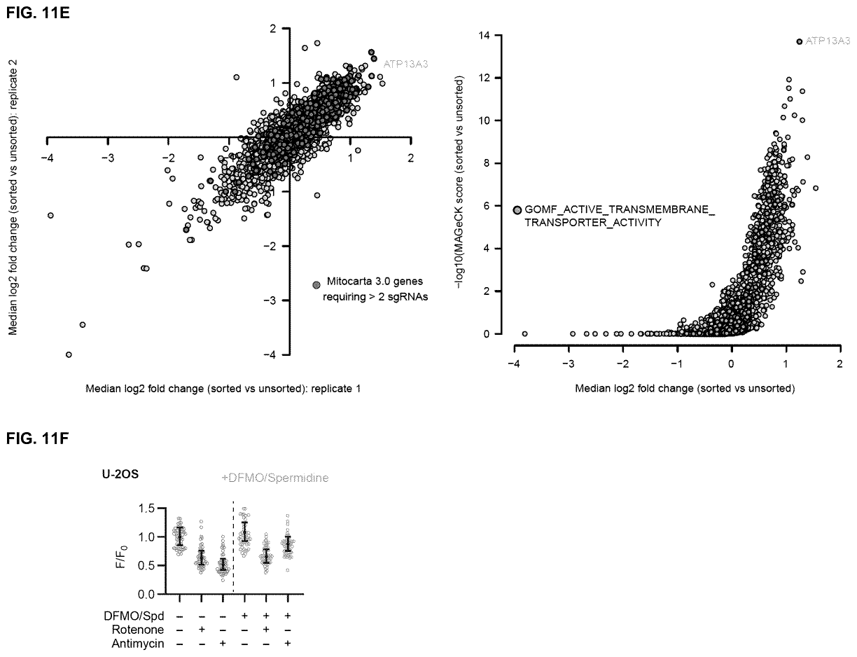

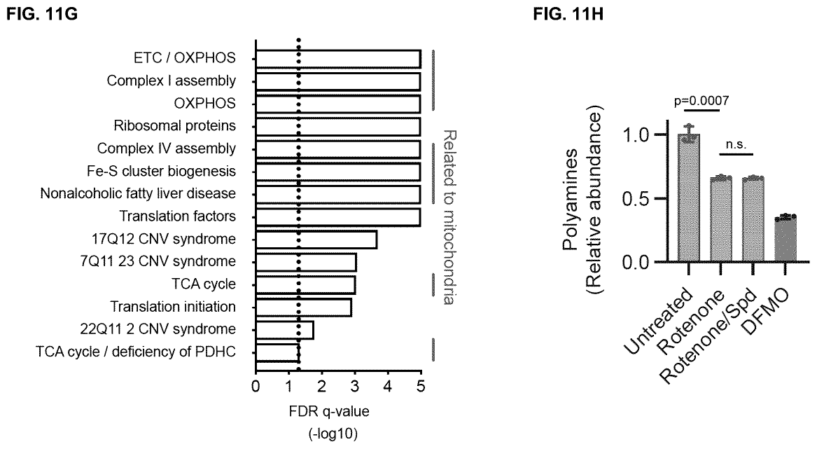

For FIG. 3B and FIG. 11H, measurements were performed using the fluorescent quantification assays (Abeam, Cambridge, England, ab239728) as per the manufacturer's instructions. The polyamine assay buffer (no added polyamine standard) was used as a background.

### Microscopy

Cells were seeded on a glass-bottom 96-well plate (Brooks Life Sciences, Chelmsford, MA, MGB096-1-2-LG-L) and imaged on a Dragonfly 505 spinning-disk confocal microscope (Andor Technology, Belfast, UK) with a piezo Z-stage (ASI), an iXon Ultra 888 EMCCD camera and appropriate filter sets. Organoids were cultured in polymer-coated chamber slides (ibidi, Gräfelfing, Germany, 80286). Pin-hole size was kept at 40 μm. Z-stacks were acquired with a step size of 0.3 to 1 μm depending on the thickness of the sample using a 100× oil immersion objective NA 1.45 (Nikon, MRD01905, pixel size 121 nm×121 nm). During the imaging, cells were kept in a humidified chamber (Okolab, Pozzuoli (NA), Italy) maintained at 37° C. and 5% (v/v) CO₂. The following laser excitation and bandpass emission filters were used: yellow fluorescent protein (YFP) (488 nm, 521/38 nm), mCherry (561 nm, 594/43 nm), and miRFP670-2 (637 nm, 620/60 nm). For each experimental condition, at least 40 randomly chosen cells were imaged in at least two independent replicates. Representative images were generated by taking an average projection of three slices centered at the focused plane.

### Flow Cytometry

For fluorescence quantification in FIG. 1A-1L and FIGS. 10A-10J, the cells were analyzed by flow cytometry (BD Biosciences, Franklin Lakes, NJ, LSR II, BD FACSDiva). At least 5,000 cells were analyzed for each experimental condition in at least two independent replicates unless otherwise noted. The flow cytometry data was then analyzed in Flowjo (Tree Star, Inc., Ashland, OR). Background fluorescence was determined for each channel in each experiment from the average signal of uninduced samples and was subtracted before intensity quantification. GraphPad Prism was used to perform statistics and generate plots. The cells underwent the same treatment as those for microscopy.

### Cell Tracking and Quantification

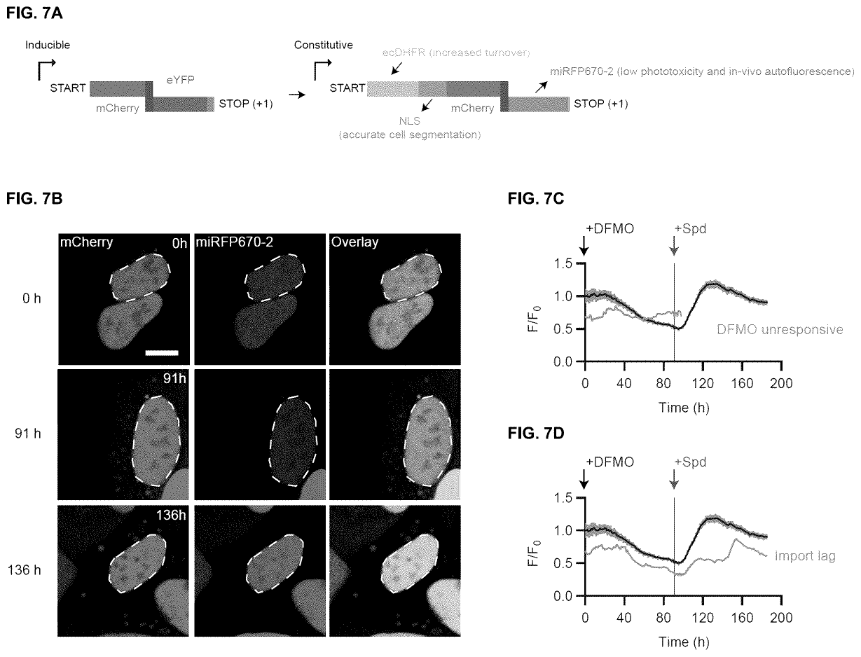

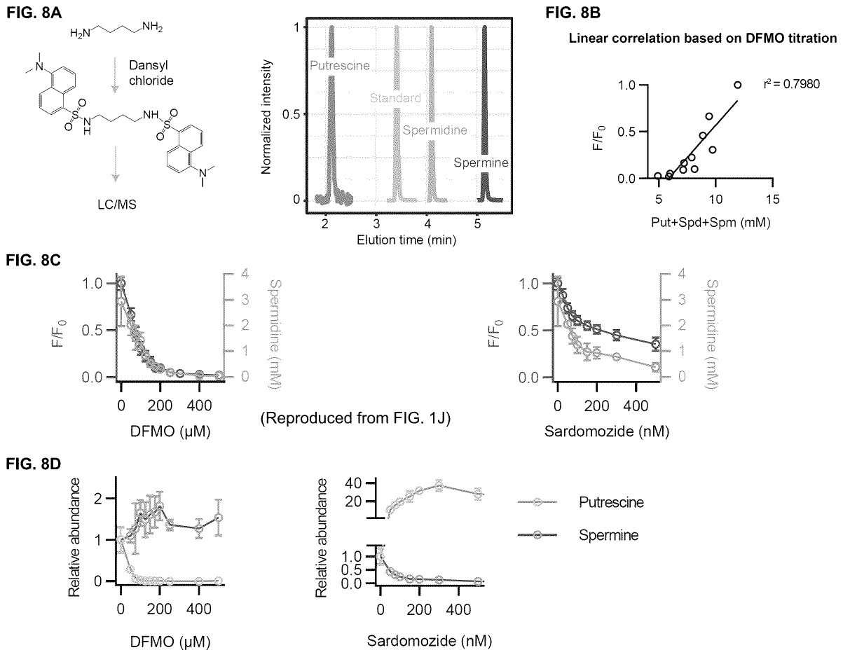

For the live tracking experiments in FIGS. 1F-1G, fluorescence was quantified directly from the images acquired using the spinning disk. F₀ is miRFP670-2/mCherry at t=0. A custom FIJI script was used to find the focal plane of Z-stacks using a normalized variance algorithm (imagejdocu.list.lu/macro/autofocus_hyperstack). Background fluorescence was computed from the average signal of 10 regions devoid of any cells. This background was calculated for each channel and subtracted before intensity quantification. The cells were segmented from background-corrected mCherry fluorescence images using Cellpose (Stringer (2021)), a deep learning-based segmentation algorithm (a pre-trained “cyto” model was directly applied without additional training). Incorrect segmented cells (a diameter of less than 25 pixels) were excluded from downstream analysis. TrackMate (Ershov (2022)) was used to perform tracking of segmented cells employing a Linear Assignment Problem (LAP) framework which uses a Jonker-Volgenant solver. In brief, a cost matrix was generated for frame-to-frame linking using a maximal allowed frame-to-frame linking distance of 75 pixels and with linking cost defined as being the distance squared. Next, the built track segments are linked using the LAP framework again, allowing the branching of a track into two sub-tracks to account for mitosis events with a maximal distance of 75 pixels. Gap-closing was also included to create a link over two spots separated by a missed Cellpose detection with a maximal distance of 150 pixels over 2 frames. These parameters were optimized by manually benchmarking the predicted cell tracks on a subset of the data. Only the longest track was retained for each identified trajectory. Cells entering or leaving the field of view were retained for analysis. Lineages with frames less than half of the total number of frames during the entire experiment duration were excluded. Incorrect segmentation or tracking events were manually filtered out. Custom MATLAB scripts were used to process the single-cell integrated intensity data. For the images shown in FIG. 7B, regions containing a particular tracked single cell were cropped, and background corrected image was used. To obtain ratiometric videos, a median filter with the Despeckle plugin was first applied to focus slices from both channels. Threshold masks were generated using OTSU (Auto Threshold plugin) for each frame and each channel. The background was then subtracted (defined for each channel as the median pixel intensity across all time frames). Pixel-by-pixel fluorescence intensities in the miRFP670-2 and mCherry channels were divided to generate a hyperstack which was then false-colored.

An increase in F (miRFP60-2/mCherry) was observed in cells immediately after the start of the image acquisition during live tracking experiments. This lasted for ˜24 hr before reaching a steady state and was likely attributed to autofluorescence and photobleaching effects. To correct this, miRFP60-2/mCherry values was corrected for DFMO-treated samples by normalizing with an exponential fit generated from miRFP60-2/mCherry values for untreated cells under similar laser conditions.

### Western Blotting

The cells were washed with ice-cold PBS and lysed with radioimmunoprecipitation assay (RIPA) lysis buffer (25 mM Tris-HCl pH 7.5 (ThermoFisher Scientific, 15567027), 150 mM NaCl (ThermoFisher Scientific, AM9760G), 1% (v/v) NP-40 (ThermoFisher Scientific, AAJ19628AP), 1% (w/v) sodium deoxycholate (Sigma-Aldrich, D6750), 0.1% (w/v) sodium dodecyl sulfate (SDS) (Bio-Rad Laboratories, Hercules, CA, 1610302)) supplemented with 1% (v/v) HALT protease and phosphatase inhibitors (ThermoFisher Scientific, 78429) and 125 U/mL Benzonase nuclease (MilliporeSigma, E1014). The lysate was homogenized and incubated on ice for 30 min with vortexing every 10 min. Cell debris were removed by centrifugation at 21,000 g for 10 min at 4° C., and lysates were mixed with 4× Bolt lithium dodecyl sulfate (LDS) sample buffer (ThermoFisher Scientific, B0007) with 100 mM Dithiothreitol (DTT) (ThermoFisher Scientific, R0861) and heated at 70° C. for 5 min. Samples were separated on a Bolt 4-12% Bis-tris polyacrylamide gel (ThermoFisher Scientific, NWO4122) and then transferred to a PVDF membrane (ThermoFisher Scientific, IB24002) using an iBlot 2 dry blotting system (ThermoFisher Scientific, IB21001). Membranes were blocked in 5% (w/v) nonfat dry skim milk (BD Biosciences, 232100) in tris-buffered saline (TBST) (ThermoFisher Scientific, AAJ60764K3) with 0.1% (v/v) Tween-20 (ThermoFisher Scientific, BP337) for 1 hr at room temperature. Membranes were incubated with primary antibodies diluted in 1% (w/v) skim milk in TBST at 4° C. overnight. After 4×TBST washes, membranes were incubated with appropriate secondary antibodies in 1% (w/v) skim milk in TBST for 1 hr at room temperature. Membranes were washed 4× with TBST, and chemiluminescence signals were detected using SuperSignal West Femto Maximum Sensitivity Substrate (ThermoFisher Scientific, 34095) with a ChemiDoc XRS+ imager (Bio-Rad Laboratories). The antibody details are as follows: rabbit anti-mCherry (Rockland Immunochemicals, Inc., Limerick, PA, s600-406-379, 1:5000), mouse horseradish peroxidase (HRP) anti-beta-actin (Abeam, ab20272, 1:50,000), anti-ODC1 (Abeam, ab193338, 1:1,000) and goat anti-rabbit HRP conjugate (Sigma-Aldrich, A0545, 1:5,000).

### RNA Sequencing and Splicing Analysis

Total RNA was extracted with PureLink RNA Mini Kit (ThermoFisher Scientific) per the manufacturer's instruction. PolyA selection and RNA library construction were performed by Novogene Corporation Inc. (Sacramento, CA) with sequencing as 150×150 base paired-end libraries using an Illumina NovaSeq 6000 (Novogene Corporation Inc.) to a depth of ≥20M reads. Reads were aligned to the human genome (hg38, annotation file GRCh GTF 38.93) and plasmid map for the reporter plasmid using the short-read alignment tool STAR (version 2.7.1a) (Dobin (2013)) with the default options. Sashimi plots for splicing analysis of the reporter were generated with ggsashimi (version 1.1.5) (Garrido-Martin (2018)).

### Polyamine Extraction for LC/MS

This protocol was adapted from previous work (Minocha (1990)). In brief, two million K562 cells were washed twice with ice-cold PBS, pelleted, and then liquid nitrogen was used to quench metabolism. The samples were transferred temporarily to dry ice before being stored in a sealed container at −80° C. The tubes were then thawed in 400 μL 0.2 M perchloric acid (PCA) and resuspended. 10 μL of 0.1 mg/mL 1,7 diamino heptane (in PCA, prepared fresh) was spiked in as a non-biological polyamine internal standard. After a brief incubation on ice (15 min) and intermediate vortexing, the cell lysate was cleared by centrifugation at 21,000 g for 10 min at 4° C., and 100 μL of the supernatant was neutralized with 200 μL of 3 M sodium carbonate (in water). Finally, 400 μL of 7 mg/mL dansyl chloride (in acetone) was added. The solution was mixed by vortexing and incubated at 60° C. for 30 min (protected from light thereafter). To quench the excess dansyl chloride, 100 μL of 100 mg/mL L-proline (in water) was added to the tube and mixed by vortexing. After 10 min of incubation at room temperature in the dark, the top phase (acetone solution) was completely removed and discarded. Next, 500 μL of toluene was added to the tube, and the solution was mixed by vortexing. Dansylated polyamines was extracted into toluene because, in the aqueous phase, they stick to the walls of the polypropylene microtubes and do not efficiently get transferred. After a brief incubation at room temperature (15 min), the tube was cleared by centrifugation at 11,000 g for 1 min, and 200 μL of the toluene phase (top) was aliquoted into a 9 mm wide autosampler glass vial, and then dried using a Mulitvap nitrogen evaporator under dark conditions. Needles were continuously adjusted to expedite the drying process. The samples were stored at −80° C. until the day of analysis. Standard solutions of polyamines (all three mixed together) were prepared at the concentrations of 0.0002, 0.0004, 0.001, 0.002, 0.005, 0.01, 0.02, 0.05, 0.1, 0.2, 0.5, 1, 2, 5, 10, 20, 50, 100, 200, 500, and 1,000 μM and were treated with the same procedure as the sample. All reagents were prepared fresh immediately before use, and all experiments were performed in at least three replicates. Due to the large number of samples, metabolite extractions were performed in multiple batches (16 individual samples at a time). To mitigate analytical bias in downstream analysis, the replicates of an experimental condition were distributed in different batches.

### Polyamine LC/MS Analysis

On the analysis day, the dried samples were centrifuged to collect all samples to the bottom of the tube, resuspended in 20 μL acetonitrile, vortexed for 5 min, and then spun down again before placing them into the autosampler. All the steps were performed at 4° C., and exposure to light was avoided. Liquid chromatography was conducted on a Vanquish Flex Binary UHPLC system (ThermoFisher Scientific) using water with 0.1% formic acid as solvent A and acetonitrile with 0.1% formic acid as solvent B. Reverse phase separation of analytes was performed on a ZORBAX RRHT Eclipse XDB-C18 column, 4.6×50 mm, 80 Å pore size, 1.8 μm particle size (Agilent Technologies, Lexington, MA, No. 927975-902; guard column, Agilent, No. 820750-903). The column oven was held at 25° C., and the injection volume was 2 μL. All injections were eluted with 70% B for 2.0 min, a gradient of 70-90% B for 1.0 min, 90-95% B for 1.0 min, 95-97.5% B for 2.0 min, 97.5-70% B for 1.0 min and 70% B for 5.0 min, with a flow rate of 1 mL/min. All mass spectrometry analyses were performed on a high-resolution Orbitrap Exploris 120 benchtop mass spectrometer (ThermoFisher Scientific) operated in positive ionization mode with a full scan range of 550-1150 m/z and top four data-dependent MS/MS scans. The orbitrap resolution is 120,000 with RF lens of 70% and static spray voltage of 3,500 V. Single-ion monitoring scans were also collected along with each method for targeted detection of the following compounds—597.2565 m/z [M+H]⁺ at 3.3 min; dansyl heptane, 555.2126 m/z [M+H]⁺ at 2.0 min; dansyl putrescine, 845.3163 m/z [M+H]⁺ at 4.0 min; dansyl spermidine, and 1135.427 m/z [M+H]⁺ at 5.0 min; dansyl spermine—with a scan width of 5.0 m/z and RT window of 1.0 min. All MS data were collected under the profile data type. Multiple acetonitrile blank and polyamine standards were interspersed throughout the run to track any technical drifts in MS signal quality.

### Polyamine LC/MS Quantification

Chromeleon 7.2.10 ES, TSQ Tune 3.1.279.9, and XCalibur 4.5 (ThermoFisher Scientific) were used for deconvolution, peak alignment, identification, and quantitation of the raw file peaks. The .raw files were imported onto MZmine 3 for quantitative analysis of dansyl heptane, dansyl putrescine, dansyl spermidine, and dansyl spermine. The following MZmine 3 modules were used: Mass detection, FTMS shoulder peak filter, ADAP chromatogram builder, Local minimum feature resolver, 13C isotope filter, Join aligner, and Same RT and m/z range gap filter. The MZmine 3 batch analysis file containing all processing parameters is attached as a .xml file in Source Data. The peak area for each polyamine was normalized by the peak area of the internal standard to represent the feature (polyamine) abundance before visualization and statistical analysis on GraphPad Prism (v9.5.1).

### Reporter Calibration

The polyamine concentrations in cells were determined by quantifying the moles of polyamines using LC/MS-based analysis and the cell volume by confocal microscopy. To estimate cell volumes, cells were mounted and imaged in the mCherry channel. Z-stacks with a step size of 0.25 μm were acquired, and the total cell volume was estimated using a custom CellProfiler pipeline (github.com/CellProfiler/tutorials/tree/master/3d_monolayer), as previously reported. At least 50 cells were analyzed, and the cell culture conditions were kept identical to those used for LC/MS analysis. The mean cell volume within each condition was then used. For LC/MS analysis, a standard curve was prepared using individual polyamines. The metabolite abundances in extracts were obtained by comparing the LC/MS data against standards fit to a linear equation. The total number of moles of a polyamine in the cell extract was then calculated from the sample concentration and the corresponding sample volume. Cellular polyamine concentrations were calculated using the total moles of a metabolite in a sample, the total number of cells per sample, and the average volume of each cell.

The exact value of eYFP/mCherry is dependent on the laser settings of the flow cytometer, and thus the polyamine concentrations was calibrated using the fold change (F/F₀) rather than the absolute value (F). To estimate the absolute cellular spermidine concentrations (mM) from fluorescence (F/F₀), the data from DFMO titration was interpolated to create a regression curve where the x-value is the F/F₀ and the y-value is the absolute spermidine concentration (mM). Fluorescence was then used to determine the approximate spermidine for ribavirin treatment. Polyamines are commonly added to commercial kits for RNA transcription and translation and hence, it was not feasible to perform these calibrations using in vitro translation of the sensor mRNA.

### Lentivirus Production for CRISPR-Cas9 Screen

15×10⁶ HEK-293T cells were seeded in T175 cm² flasks in DMEM (ThermoFisher Scientific, #12430054) supplemented with 10% fetal bovine serum (GeminiBio, West Sacramento, CA, #100-106). After 24 hr, the media was changed to 20 mL viral production medium: Iscove's Modified Dulbecco's Medium (IMDM, ThermoFisher Scientific #1244053) supplemented with 20% inactivated fetal serum (GeminiBio, #100-106). At 32 hr post-seeding, cells were transfected with a mix containing 76.8 μL Xtremegene-9 transfection reagent (Sigma-Aldrich, #06365779001), 3.62 μg pCMV-VSV-G (Addgene plasmid #8454) (Stewart (2003)), 8.28 μg psPAX2 (a gift from Didier Trono, Addgene plasmid #12260), and 20 μg sgRNA/Cas9 plasmid and Opti-MEM (ThermoFisher Scientific, #11058021) to a final volume of 1 mL. Media was changed 16 hr later to 55 mL fresh viral production medium. The virus was collected at 48 hr post-transfection, filtered through a 0.45 μm filter, aliquoted, and stored at −80° C. until use.

### CRISPR-Cas9 Screen

A genome-wide lentiviral sgRNA library (Inglis (2023)) in a Cas9-containing vector comprising 97,888 unique sgRNA sequences with ˜5 sgRNAs per target was used to transduce 600×10⁶ K562 cells to achieve an MOI<1 (30-50% transduction efficiency) and ˜1,000-fold library coverage. Briefly, polybrene (10 μg/mL final concentration) and virus were mixed with the cells (2.5×10⁶ cells/mL final density) and distributed into individual wells in 6-well plates. The plates were centrifuged at 1,126 g for 45 min at 37° C. and transferred to an incubator. After 8 hr, the cells were pelleted, the virus was removed, and the cells were resuspended in fresh growth medium and transferred to T225 cm² flasks (250,000 cells/mL final density). After 36 hr, the cells were collected and reseeded in fresh growth medium (200,000 cells/mL final density), and puromycin was added (3 μg/mL final concentration). After 3 days, the cells were collected, and transduction efficiency was determined by comparison of cell survival of transduced cells relative to control cells (untransduced and unselected). The cells were passaged every 2 days (0.2×10⁶ cells/mL) for 3 days before reporter induction using doxycycline for 24 hr. At the screen endpoint, the cell pellets were collected from flasks representing 1,000-fold library coverage of unsorted cells and frozen at −80° C. The induced cells were sorted, and the cell pellets were collected.

### Sequencing Library Preparation

Genomic DNA (gDNA) was extracted from cell pellets of 40-50×10⁶ cells using the QIAamp DNA Blood Maxiprep Kit (QIAGEN, N.V., Hilden, Germany, #51192) according to manufacturer's instructions with minor modifications: QIAGEN Protease was replaced with 500 μL of a 10 mg/mL solution of Proteinase K (MilliporeSigma, #3115879001) in water; cells were lysed overnight; centrifugation steps after Buffer AW1 and AW2 were performed for 2 min and 5 min, respectively; 1 mL of water preheated to 70° C. was used to elute gDNA (5-min incubation), followed by centrifugation for 5 min. gDNA was quantified using the Qubit dsDNA HS Assay kit (ThermoFisher Scientific, #Q32851).

All PCR reactions were performed in 50 μL reactions using ExTaq Polymerase (Takara Bio #RR001B) with the following program and primers:

| | | | | |
|---|---|---|---|---|
| 1 | cycle | 95° C. | 5 | min |
| 28 | cycles | 95° C. | 10 | seconds |
| | | 60° C. | 15 | seconds |
| | | 72° C. | 45 | seconds |
| 1 | cycle | 72° C. | 5 | min |

| | | | |
|---|---|---|---|
| 1 | cycle | 4° C. | hold |

Forward Primer
(SEQ ID NO: 11)
AATGATACGGCGACCACCGAGATCTACACCCCACTGACGGGCACCGGA
Reverse Primer
(SEQ ID NO: 12)
CAAGCAGAAGACGGCATACGAGATCnnnnnnTTTCTTGGGTAGTTTGC
AGTTTT

where “nnnnnn” denotes the barcode used for multiplexing.

For all samples, 1, 3, or 6 μg of gDNA was initially amplified for 28 cycles in 50 μL test PCR reactions. Subsequently, an additional 23.33 reactions (23.33×50 μL reactions) were performed using 6 μg per reaction (140 μg gDNA). The reactions were pooled, and 100 μL of each reaction was purified using Select-a-Size DNA Clean and Concentrator (Zymo Research, Irvine, CA, #D4080), eluted with 15 μL water, and quantified using the Qubit dsDNA HS Assay kit before sequencing for 50 cycles on an Illumina Hiseq 2500 using the following primers:

Read 1 sequencing primer
(SEQ ID NO: 13)
GTTGATAACGGACTAGCCTTATTTAAACTTGCTATGCTGTTTCCAGCA
TAGCTCTTAAAC
Index sequencing primer
(SEQ ID NO: 14)
TTTCAAGTTACGGTAAGCATATGATAGTCCATTTTAAAACATAATTTT
AAAACTGCAAACTACCCAAGAAA

### CRISPR Screen Data Analysis

Sequencing reads were trimmed and mapped to the sgRNA library using Bowtie version 1.0.0 (Langmead (2009)) and counted. Data was analyzed using MAGeCK version 0.5.9.5 (Li (2014)) with the following parameters: gene test false discovery rate (FDR) threshold of 0.05; FDR method for p-value adjustment; median as the gene-level scoring metric; sgRNAs targeting intergenic regions as control sgRNAs. Pre-ranked gene set enrichment analysis (GSEA) (Subramanian (2005)) was performed with version 4.1.0 using the Human_Gene_Symbol_with_Remapping_MSigDB.v2023.1.Hs chip and the c2.cp.kegg_medicus.v2023.2.Hs.symbols (c2.cp.wikipathways.v2023.2.Hs.symbols (Agrawal (2024)) gene set. The enrichment statistic was set to classic, normalization was set to meandiv, and the maximum score was used as the collapsing mode. Data was visualized using R version 4.2.1 corrplot package version 0.92 (Wei & Simko (2021)) and base graphics, and GraphPad Prism version 10.

### Example 2. Sensor Design and Validation

To design a polyamine sensor, a polyamine-responsive OAZ1 frameshifting element, devoid of the catalytically active domain, was embedded between two fluorescent proteins, mCherry and eYFP. eYFP sequence lacking an ATG start codon was cloned in the +1-frame downstream of mCherry (FIG. 1B). Ribosomes translating in the mCherry frame encounter a stop codon, and eYFP production requires a +1 frameshift. eYFP was cloned downstream of mCherry because its coding sequence lacks in-frame methionine codons within its N-terminus, and translation initiation at the encoded methionines should not produce a fluorescent protein (Fages-Lartaud (2022)). The mCherry fluorescence signal provides an internal control, allowing normalization for cell-to-cell variations in transduction efficiency, mRNA stability, and translational status. The frameshift efficiency could be monitored via the relative fluorescence (F=eYFP/mCherry fluorescence) and reports on cellular polyamine levels.

Various segments of the OAZ1 gene were examined, and a 170-nucleotide region was identified that retains polyamine-responsive frameshifting (FIGS. 2B-2I, see Example 3). To prevent accumulation of fluorescent proteins over time, which could confound the ratiometric readout, the reporter was expressed under a doxycycline-inducible promoter, ensuring that the fluorescence signal reports on the polyamine levels during a brief, well-defined time window. Expression of this reporter in U-2OS cells resulted in both mCherry and eYFP proteins, demonstrating efficient ribosomal frameshifting (FIGS. 1C-1D). Western blot analysis confirmed the production of the mCherry-eYFP fusion protein with a molecular weight consistent with a +1 ribosomal frameshift event (FIG. 1C). Insertion of a stop codon immediately after the mCherry coding region and before the OAZ1-derived frameshifting region effectively eliminated the eYFP fluorescence signal, validating that eYFP expression was not due to alternative translation initiation within the eYFP sequence and that the eYFP signal did not arise from spectral bleed-through from the mCherry fluorescence (FIGS. 4A-4B). Furthermore, RNA sequencing confirmed that the RNA transcribed from the reporter construct corresponded to the DNA sequence and did not exhibit any unexpected transcriptional or post-transcriptional processing events that could account for the eYFP production independent of frameshifting (FIG. 4C). Reporter expression did not trigger measurable ODC1 degradation (FIG. 3A) or alter cellular polyamine levels (FIG. 3B), ensuring that sensor expression does not perturb the endogenous polyamine homeostasis.

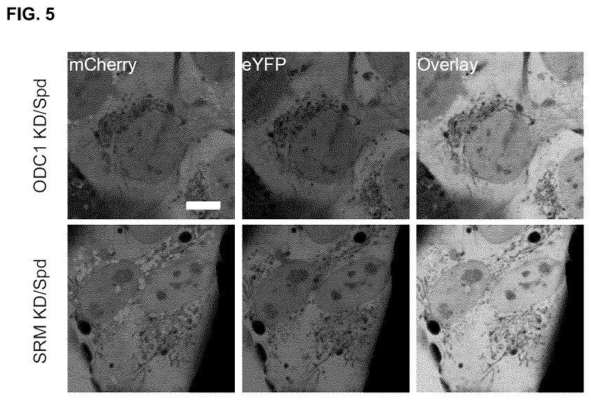

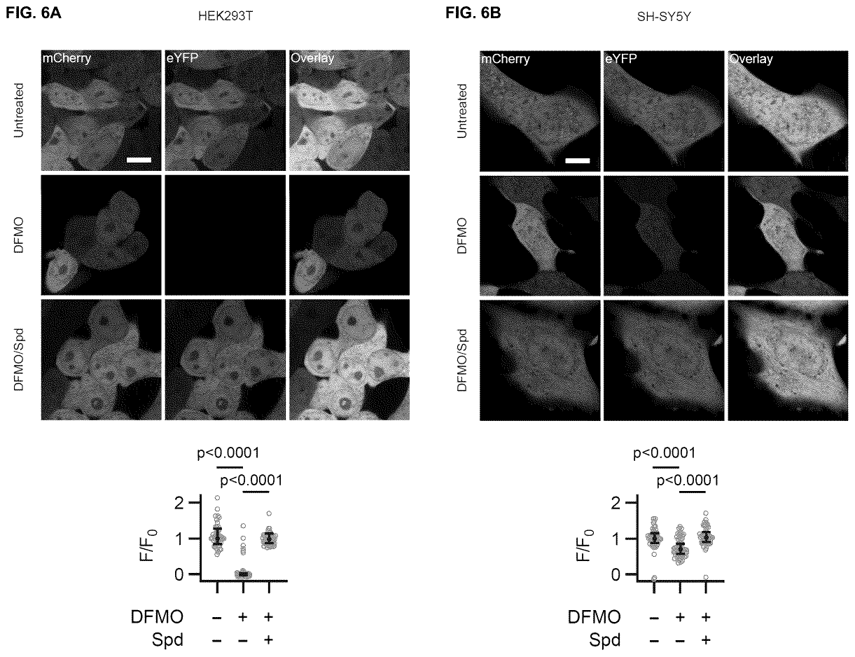

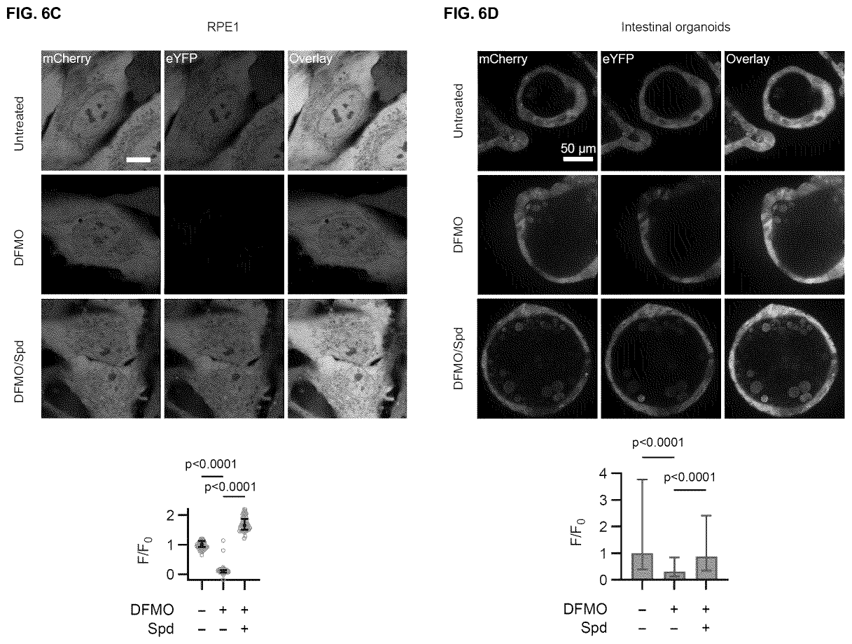

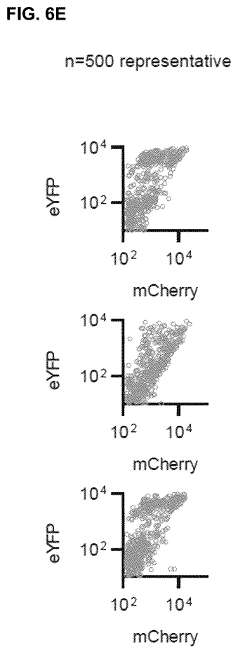

Depletion of cellular polyamines using difluoromethylornithine (DFMO), a polyamine synthesis inhibitor (McCann & Pegg (1992)) led to a marked reduction in eYFP and mCherry levels, consistent with the role of polyamines in protein translation (Dever & Ivanov (2018)) (FIG. 1D; DFMO). Notably, the decrease in eYFP signal was more pronounced than that for mCherry (92% and 54% respectively, compared to untreated controls). Frameshifting efficiency was quantified as the ratio of eYFP to mCherry fluorescence (F), normalized to that for untreated cells (F₀) to account for variations in fluorescence imaging conditions. This normalized ratio (F/F₀) decreased five-fold (from 1.00 to 0.18) following DFMO treatment (1 mM for 72 hr) but was restored (F/F₀≈1.2) upon spermidine supplementation (5 μM spermidine for 14 hr, FIGS. 1D-1E). Importantly, the stimulatory effect of polyamines on frameshifting was specific to the OAZ1 sequence. A control construct harboring the −1 ribosomal frameshift motif derived from SARS-CoV-2 exhibited pronounced frameshifting under basal conditions, but its frameshifting efficiency was refractory to polyamine depletion (FIGS. 4D-4E). Since polyamines can be oxidized by amine oxidases present in the serum, experiments involving polyamine supplementation and corresponding controls were performed in the presence of aminoguanidine, which potently inhibits amine oxidase activity (Holbert (2020)). Similar to DFMO treatment, genetic perturbations to the polyamine biosynthetic pathway, such as by ODC1 or SRM knockdown also reduced F/F₀ for our reporter, which could be reversed by spermidine supplementation (FIGS. 1F-1G, FIG. 5). This polyamine dependent frameshifting (F/F₀) was observed in other human cell lines (HEK293T, SH-SY5Y, and RPE1) as well as primary intestinal organoids derived from C57BL/6 mice (FIGS. 6A-6E), demonstrating the broad applicability of this reporter across different cellular contexts.

Next, the fluorescence readout was calibrated against liquid chromatography-mass spectrometry (LC-MS) based polyamine quantification (see “Reporter Calibration” in Example 1). The polyamine levels were modulated by titrating DFMO, and the fluorescence readout from individual cells was compared to ensemble polyamine measurements using LC-MS under equivalent treatments (FIG. 8A). This analysis showed that the reporter read-out (F/F₀), measured at single-cell resolution, correlated strongly with the average polyamine concentration measured by mass spectrometry (r²=0.79) (FIG. 8B). Interestingly, F/F₀ showed striking correspondence with the cellular spermidine concentration (r²=0.96), but not with putrescine or spermine (FIG. 1J, FIGS. 8C-8D). Similar results were obtained when cellular polyamine levels were changed by titrating sardomozide, a polyamine biosynthesis inhibitor that targets AMD1 (FIGS. 8C-8D). These observations are consistent with previous studies showing that the frameshifting in OAZ1 mRNA in cells is primarily stimulated by spermidine (Ray (2014)). Although putrescine may also induce OAZ1 frameshifting, it requires ˜10-20-fold higher concentrations (Rato (2011)) which are unlikely to be encountered under normal growth conditions (also see Example 6). Likewise, while spermine can also promote OAZ1 frameshifting (Rato (2011)), its biochemically available concentration is substantially (about 5-10 fold) lower than that of spermidine (Al-Habsi (2022), Marton & Pegg (1995), Watanabe (1991)). In agreement with this notion, prolonged treatment with a high dose of DFMO (500 μM for 3 days) reduced F/F₀ to background levels (F/F₀=0.02, representing a 50-fold decrease) despite unchanged spermine levels, as measured by mass spectrometry (FIG. 1J, FIG. 8D). Collectively, these results emphasize that under normal physiological conditions, this sensor primarily reports on spermidine, which is the most abundant polyamine in mammalian cells.

The reporter disclosed herein is derived from a native feedback regulatory motif, and therefore, is expected to be responsive in the regime that the cells are likely to encounter. The frameshift efficiency (F/F₀) monotonically increased with cellular spermidine concentration, spanning a 100-fold range (from ˜40 μM to 4 mM) (FIG. 1K) underscoring the high sensitivity and large dynamic range of the sensor. To substantiate the quantitative capabilities of the reporter, cells were treated with ribavirin, which was recently identified to activate SAT1 and deplete cellular polyamines (Tate (2019)). Remarkably, the cellular polyamine concentration estimated using the reporter closely matched those determined by conventional mass spectrometry-based analysis (FIG. 1L). Notably, while mass spectrometry required over ˜1 million cells, the present reporter enabled measurements at single-cell resolution. Moreover, the present reporter exhibited substantially low technical variability with the coefficient of variations (CV) of only ˜3% in measurements across 3 days (FIGS. 9A-9B). Collectively, these results show that the present reporter provides a sensitive and quantitative readout of cellular polyamine levels in single living cells.

This reporter system also allows performing real-time monitoring of polyamine levels in individual cells over a long duration. To further enhance the reporter's performance for these tracking experiments, a degron tag (Iwamoto (2010)) was incorporated, which ensured rapid protein turnover and mitigated the reduction in the dynamic range associated with the build-up of the fluorescent signal. eYFP was also replaced with a near-infrared fluorescent protein, miRFP670-2 (Zhang (2023)), substantially reducing the phototoxicity associated with long-term live-cell imaging (FIG. 7A, see Example 5). Previous experiments have indicated that DFMO-treated cells are primed for polyamine import (Stewart (2023)). To directly test this hypothesis, the kinetics of polyamine depletion by DFMO and subsequent rescue with spermidine was probed (FIGS. 1H-1I, FIGS. 7B-7D). DFMO treatment gradually reduced the cellular polyamine content over ˜90 hr, while spermidine supplementation in DFMO-treated cells restored intracellular polyamine levels to the baseline. Interestingly, an initial overshoot of F/F₀ relative to the baseline upon spermidine supplementation was observed, which persisted for ˜40 hr.

Accordingly, a novel genetically encoded fluorescent reporter that enables quantitative measurement of polyamine levels in living cells at single-cell resolution is established. This reporter system, based on the endogenous polyamine-responsive frameshift motif found in the OAZ1 gene, provides a real-time readout of intracellular polyamine concentrations with high sensitivity and a wide dynamic range. The simplicity of the assay greatly accelerates the measurement process, eliminating the need for extraction from native cellular context and extensive chemical derivatization. The single-cell resolution of the sensor enables high-throughput screens and identifying new modulators of polyamine homeostasis. Moreover, the ability to track polyamine levels within the same sample allows direct pharmacokinetic studies of polyamine-targeting therapies, including potentially in live animals.

### Example 3. Design and Characterization of a Polyamine Reporter

The human OAZ1 gene consists of 685 coding nucleotides with an in-frame UGA stop codon located at the nucleotides 205-207 (NCBI NM_004152.3). Production of full-length catalytically active OAZ1 requires a +1 ribosomal frameshift at the stop codon. The sequence spanning 1-275 nucleotides in OAZ1 mRNA was used as the starting point for reporter design as it does not bind ODC1, lacks the catalytically active C-terminal domain (Matsufuji (1995), Rom & Kahana (1994), Matsufuji (1996)), but is expected to retain the frameshift activity. This truncated version contains a 60-nucleotide long frameshift stimulatory RNA pseudoknot three nucleotides downstream of the slippery site (UCC UGA), potentially acting as a mechanical stressor to force the translating ribosome into a new reading frame (Atkins (2016)). The second mutant contained only nucleotides 106-275, cloned downstream of an ATG start codon. Previous ribosome profiling experiments have shown that a sizable fraction of ribosomes is paused in this region, suggesting a putative role in translational repression (Mizrahi (2018)). This construct (106-275) also avoids potential interactions of the translated N-terminal OAZ1 protein with the mitochondrial import machinery (Gandre (2003)). A third construct (185-275) consisting of the last few codons in ORF1, the frameshift site, and pseudoknot was cloned based on the evolutionary conservation of the nucleotide sequence 5′ to the shift site across homologs of vertebrates antizyme genes (Ivanov & Atkins (2007)). A mutant lacking the pseudoknot (1-216) was also generated to measure its contribution to polyamine-mediated frameshifting. These regions were individually cloned between mCherry and YFP fluorescent proteins, under a doxycycline-inducible promoter. Each construct was stably integrated into U-2OS cells and was evaluated for (i) its ability to induce ribosomal frameshift, and (ii) its responsiveness to cellular polyamine levels as determined by reduction in frameshifting upon treatment with DFMO (an irreversible inhibitor of ODC1) and its rescue after adding exogenous spermidine (FIGS. 2A-2I). All constructs retained the ability to induce polyamine-responsive frameshift, albeit to varying degrees. The presence of the pseudoknot significantly enhanced the frameshifting efficiency. The 106-275 mutant was chosen for the polyamine sensor since this sequence is relatively short, retains high frameshifting efficiency, and produces a substantial change in the eYFP:mCherry fluorescence ratio between DFMO treatment and spermidine supplementation.

### Example 4. Genetic Modifiers of Polyamine Import

The mammalian polyamine transport pathway remains poorly understood (Miller-Fleming (2015)). One proposed polyamine import mechanism involves lysosomal P5B-ATPases, ATP13A2, and/or ATP13A3 (van Veen (2020), Azfar (2022)), that release lysosomal polyamines in the cytoplasm. Several other proteins have also been proposed to participate in polyamine uptake but their precise roles remain to be established (Eom (2022), Daigle (2009), Aouida (2010), Uemura (2010), Belting (2003)). The present reporter was used to develop a polyamine import assay that reports on import activity at a single-cell level (FIG. 10A). In brief, cells were treated with DFMO to block endogenous polyamine biosynthesis, rendering them dependent on exogenously supplied spermidine. Polyamine import from the extracellular medium would restore cellular polyamine concentration, which can be quantitatively read out using the reporter. As a proof-of-concept, the effect of the drug AMXT-1501, a lipophilic polyamine mimetic that potently blocks polyamine transport (Burns (2009)), was examined. Treatment with AMXT-1501 alone did not affect cellular polyamine levels (F/F₀) indicating that under normal growth conditions, de novo polyamine synthesis is sufficient to maintain homeostatic levels (FIG. 10B, FIG. 11A). However, co-treatment of cells with AMXT-1501 and DFMO led to a striking 10-fold reduction in F/F₀, which could not be rescued by exogenous spermidine supplementation (FIG. 10B, FIG. 11A). These results indicate that this assay allows monitoring of polyamine import in single cells, contributing to identification of genetic and/or pharmacological modulators of this process.

Leveraging this assay, a genome-wide CRISPR-Cas9 knockout screen was performed to identify factors that influence polyamine uptake (FIG. 11B). Clonal K562 cells that express the polyamine sensor were generated and transduced with a pooled library of CRISPR single guide RNAs (sgRNAs) targeting 20,000 protein-coding genes with an average of 5 sgRNAs per target (Inglis (2023)). Following library transduction, cells were co-treated with DFMO and spermidine. Fluorescence-activated cell sorting (FACS) was used to isolate cells based on eYFP/mCherry fluorescence ratio. It is expected that in the presence of DFMO and spermidine, the import-competent cells would maintain their polyamine levels, while genetic knockouts that result in deficient spermidine import would have reduced polyamine content (FIG. 11B).

ATP13A3, a known polyamine importer, emerged as the top hit from the screen (FIG. 10C, FIG. 11E). Strikingly, knockout of ATP13A2, another P-type ATPase associated with polyamine import and Parkinson's disease, did not decrease polyamine import (FIG. 10D), despite being expressed at levels comparable to those of ATP13A3 in these cells (FIG. 11C). Moreover, knockout of other proposed polyamine transporters (for example, SLC3A2, SLC12A8, SLC7A2, and GPC1) had no discernible effect on polyamine import in our screen (FIG. 10D). Interestingly, ATP13A3 knockout was sufficient to abrogate spermidine import phenocopying the effects of AMXT-1501 in these cells (FIG. 10F). This effect was consistent across other cell lines, including U-2OS and RPE1 cells (FIGS. 10G-10I, FIG. 11D). The complete cessation of import upon ATP13A3 knockout underscores that it is an essential and indispensable component of the spermidine import pathway across various cell types.

The screen also identified ˜180 putative modifiers of spermidine uptake (median log 2 fold change>0.75 with FDR<0.05). These hits included known genes related to polyamine metabolism, including AZIN1 (antizyme inhibitor 1) and SMS (spermine synthase) (FIG. 10E). The inhibition of polyamine import upon AZIN1 knockout is consistent with the known feedback repression of polyamine uptake caused by the stabilization of the antizyme family proteins upon AZIN1 depletion (Keren-Paz (2006)). Likewise, SMS is responsible for the conversion of spermidine to spermine, and its loss leads to spermidine accumulation that inhibits polyamine transport activity (Kaouass (1998)).

Interestingly, several components of the mitochondrial electron transport chain (ETC) emerged as predominant modifiers of polyamine import (FIG. 11F). Gene set enrichment analysis confirmed a significant enrichment of ETC-related genes among the top hits from the screen (FDR q-value<0.0001) (FIG. 11G). To validate this cross-talk between ETC inhibition and polyamine import, U-2OS cells were treated with pharmacological inhibitors of ETC. Treatment with rotenone, a plant metabolite that potently inhibits complex I of the ETC, substantially decreased cellular polyamine levels (FIG. 10J). A similar decrease was observed following treatment with antimycin A, an inhibitor of complex III (FIG. 10J). Notably, reductions in polyamine levels due to rotenone or antimycin A treatment could not be compensated by exogenous supplementation with spermidine, confirming import deficits (FIG. 10J). This reduction in polyamine levels and deficient import were further verified using an ensemble enzymatic assay polyamine measurement (FIG. 11H). Other genes whose knockout reduced spermidine uptake included the subunits of the endoplasmic reticulum signal recognition particle (SRP) receptor subunits (SRPRA and SRPRB), the retromer complex (VPS35, VPS26A, and VPS29), CARNMT1 (anserine synthase), PUS3 (a tRNA pseudouridine synthase) and UFM1 (involved in the UFMylation pathway) (FIG. 10E). Altogether, the ability to quantitatively monitor polyamines at a single-cell resolution facilitated the identification of modifiers of polyamine uptake and raises questions about the potential mechanisms linking mitochondrial function to polyamine transport.

Abnormalities in polyamine transport are implicated in several devastating diseases. For example, dysregulation of polyamines is associated with Parkinson's disease and cancer, driving interest in therapeutically targeting this pathway to restore balance. Polyamine indicators disclosed herein overcomes these technical limitations and helps elucidate the molecular details of polyamine transport and its feedback regulation, potentially advancing therapy development.

The genome-wide CRISPR knock-out screen disclosed herein confirmed the essential role of ATP13A3 in polyamine import across different cell types and uncovered several new regulators of spermidine uptake. Strikingly, several identified polyamine import modifiers, such as VPS35 and subunits of ETC complex 1 (e.g., NDUFS1) are associated with Parkinson's disease (Williams (2017), Gonzilez-Rodriguez (2021), Small & Petsko (2015)). Likewise, rotenone exposure recapitulates the key pathological and clinical features of Parkinson's disease in animal models (Tanner (2011)). Given that loss of function mutations in the polyamine importer, ATP13A2, cause juvenile-onset Parkinson's disease (van Veen (2020)), these findings reinforce the link between polyamine homeostasis and Parkinson's pathogenesis. Notably, the synthesis of several ETC proteins requires spermidine-mediated post-translational modification (hypusination) of translation factor eIF5A (Puleston (2019)) further substantiating the cross-talk between mitochondrial respiration and polyamine homeostasis.

One possible explanation for the inhibitory effects of ETC blockade on polyamine levels is the disruption of one-carbon metabolism. ETC inhibition impairs the tricarboxylic acid (TCA) cycle and leads to depletion of aspartate (Birsoy (2015), Sullivan (2015)). This aspartate deficiency may activate ATF4 (Krall (2021)) and redirect one-carbon metabolism towards transsulfuration (Bao (2016)), thereby limiting the availability of S-adenosylmethionine (SAM) for polyamine synthesis. ETC inhibition also suppresses de novo purine synthesis (Wu (2023)). AMD1, a rate-limiting enzyme in polyamine synthesis, shares a strong co-dependency with both de novo purine synthesis and oxidative phosphorylation enzymes (the Cancer Dependency Map (Ghandi (2019)), suggesting a significant metabolic cross-talk between de novo purine synthesis, the ETC, and polyamine metabolism. It is speculated that inhibition of polyamine biosynthesis is an adaptive response to help cells cope with compromised ETC function. Decreased polyamine synthesis will suppress global protein translation rates (Dever & Ivanov (2018)) to maintain homeostasis under an energetically demanding condition.

The present screen also identified the retromer complex and the signal recognition particle (SRP) receptor as modulators of spermidine uptake. The retromer complex transports recycling cargo from endosomes to the plasma membrane and trans-Golgi network (Banos-Mateos (2019)). ATP13A3, a recycling endosome resident protein (Cho (2022)), partially re-localizes to the plasma membrane upon polyamine depletion (Sekhar (2022)). Loss of retrograde transport may reduce the polyamine-regulated recycling of ATP13A3 to the plasma membrane, affecting spermidine import. Similarly, the SRP pathway is essential for the co-translational delivery of membrane and secretory proteins to the endoplasmic reticulum for translation (Luirink & Sinning (2004)). Loss of SRP receptor subunits interferes with SRP binding (Kellogg (2022)) and can trigger a quality control mechanism called the Regulation of Aberrant Protein Production (RAPP), leading to mRNA degradation of SRP-targeted proteins (Karamyshev (2014)). As a membrane protein, ATP13A3 is also expected to be directed to the endoplasmic reticulum by SRP and is thus susceptible to defects in SRP.

Polyamines are at the crossroads of a multitude of cellular processes from gene expression and protein synthesis to cell proliferation and death. Polyamine levels decline with age, and dietary polyamine supplementation in model organisms improves memory (Gupta (2013)), cardiac health (Eisenberg (2016)), reproductive aging (Zhang (2023)), and lifespan (Eisenberg (2016), Eisenberg (2009)). There is resurgent interest in these metabolites but their precise biochemical functions in the cell remain elusive. Polyamine regulatory pathways are also replete with intriguing biochemical regulation that defy common principles such as stop codon readthrough and frameshifting (in OAZ1) (Matsufuji (1995)), translation repression (of SAT1) (Perez-Leal (2012)), ubiquitin-independent proteasomal degradation (of ODC1) (Coffino (2001)), and an essential posttranslational modification, hypusination, found only on a single protein, eIF5A (Park & Wolff (2018)). The availability of easy-to-use polyamine measurement technology disclosed herein provides a powerful platform to facilitate discoveries into the biology of these abundant and mysterious metabolites and may suggest novel strategies for therapy development and disease prevention.

### Example 5. Measuring Temporal Changes in Intracellular Polyamine Levels

A key advantage of the present reporter is that it allows one to assess changes in polyamine levels within the same cell over time. A set of additional features can be engineered in the reporter to enable long-term live-cell imaging (FIG. 7A). First, eYFP was replaced with the far-red miRFP670-2 to reduce phototoxicity associated with long-term blue light illumination. miRFP670-2 also lacks in-frame methionine codons within its N-terminus and has minimal spectral overlap with mCherry. Second, the DHFR (dihydrofolate reductase) tag was used for protein destabilization (Iwamoto (2010)) to improve the response kinetics. The small molecule ligand, trimethoprim (TMP), was constitutively added to stabilize the DHFR fusion protein. TMP concentration was empirically optimized to facilitate rapid turnover of the fusion protein while maintaining an appropriate signal-to-noise ratio. Third, a nuclear localization signal was incorporated in the reporter to aid cell segmentation for automated tracking by demarcating the nucleus.

These modifications allow monitoring of polyamine levels in live cells in response to the drug DFMO over 8 days using time-lapse microscopy. A continuous decline in the frameshift efficiency, F/F₀ (F=miRFP670-2/mCherry, F₀ is the mean F at t=0) was observed, after DFMO treatment which lasted for approximately 90 hr until it reached a steady low value of F/F₀≈0.5 (FIGS. 1H-1I). Spermidine addition to cells in this low-polyamine state triggered an increase in F/F₀, observable within approximately 6 hr after supplementation. At around 40 hr after spermidine addition, F/F₀ peaked at a value of about 1.2, about 20% higher than that observed in untreated cells. Subsequently, F/F₀ gradually started returning to the basal levels and stabilized after approximately 30 hr. These data indicate that inhibition of polyamine biosynthesis using DFMO elicits a compensatory increase in polyamine uptake, and results in a temporary excess. The increased uptake is under negative feedback regulation, leading to a gradual return to basal homeostatic levels. This single-cell analysis also revealed significant cell-to-cell heterogeneity in this response. A small subset of cells in the population escaped the effects of DFMO for as long as approximately 90 hr (FIG. 7C). Some cells showed an extended delay in transitioning out of the polyamine-low state upon spermidine addition (FIG. 7D). Note that the F/F₀ in these experiments (measured using miRFP670-2 and mCherry fluorescence) was not directly comparable to the results in FIGS. 1H-1I due to differences in fluorescent proteins and protein-turnover kinetics.

### Example 6. Comparing the Effect of Different Polyamines

Titrations with sardomozide, an inhibitor of AMD1 that leads to excessive putrescine accumulation while depleting spermidine and spermine, revealed that F/F₀ correlated with intracellular spermidine content but not with putrescine (FIG. 8B-8D). When cells were treated with a relatively high dose of sardomozide (500 nM), the cellular putrescine levels substantially increased by ˜25-fold, but the frameshift efficiency, F/F₀, decreased to 0.35 (F/F₀=1 for untreated cells). These results suggest that putrescine does not efficiently stimulate +1 ribosomal frameshifting on OAZ1 mRNA, which is consistent with a previous study indicating that putrescine can only induce comparable +1 ribosomal frameshifting on OAZ1 at concentrations ˜10-20 fold higher than spermidine (Rato (2011)). This observation also explains the higher offset in F/F₀ observed in sardomozide-treated cells compared to what would be expected based on DFMO titrations. Therefore, under typical physiological conditions, putrescine is likely to only have a modest effect on the present reporter readout.

Additional information of the experiments described in Examples 1-6 can be found in (i) Sharma et al., Genetically encoded fluorescent reporter for polyamines, bioRxiv [Preprint]. 2024 Nov. 17:2024.08.24.609500, and (ii) Sharma et al., Genetically encoded fluorescent reporter for polyamines, Nat Commun. 16(1):4921 (2025) (including Supplementary Information, Description of Additional Supplementary Information, Supplementary Data 1, Supplementary Data 2, and Supplementary Data 3), the contents of which are incorporated herein by reference in their entirety.

### REFERENCES

1. Pegg A E, Functions of Polyamines in Mammals, J Biol Chem. 291(29):14904-12 (2016).
2. Puleston et al., Polyamine metabolism is a central determinant of helper T cell lineage fidelity, Cell 184(16):4186-202 (2021).
3. Madeo et al., Spermidine in health and disease, Science 359(6374):eaan2788 (2018).
4. Hofer et al., Mechanisms of spermidine-induced autophagy and geroprotection, Nat Aging 2(12):1112-29 (2022).
5. van Veen et al., ATP13A2 deficiency disrupts lysosomal polyamine export, Nature 578(7795):419-24 (2020).
6. Snyder & Robinson, Recessive sex-linked mental retardation in the absence of other recognizable abnormalities. Report of a family, Clin Pediatr (Phila) 8(11):669-74 (1969).
7. Ziegler et al., Bi-allelic variants in DOHH, catalyzing the last step of hypusine biosynthesis, are associated with a neurodevelopmental disorder, Am J Hum Genet. 109(8):1549-58 (2022).
8. Casero et al., Polyamine metabolism and cancer: treatments, challenges and opportunities, Nat Rev Cancer 18(11):681-95 (2018).
9. Holbert et al., Polyamines in cancer: integrating organismal metabolism and antitumour immunity, Nat Rev Cancer 22(8):467-80 (2022).
10. Miller-Fleming et al., Remaining Mysteries of Molecular Biology: The Role of Polyamines in the Cell, J Mol Biol. 427(21):3389-406 (2015).
11. Tate et al., Ribavirin Induces Polyamine Depletion via Nucleotide Depletion to Limit Virus Replication, Cell Rep. 28(10):2620-33 (2019).
12. Minocha et al., High-performance liquid chromatographic method for the determination of dansyl-polyammines, J. Chromatogr. A 511, 177-83 (1990).
13. Seiler, Use of the dansyl reaction in biochemical analysis, Methods Biochem Anal. 18:259-337 (1970).
14. Fong et al., The appearance of an ornithine decarboxylase inhibitory protein upon the addition of putrescine to cell cultures, Biochim Biophys Acta. 428(2):456-65 (1976).
15. Matsufuji et al., Autoregulatory frameshifting in decoding mammalian ornithine decarboxylase antizyme, Cell 80(1):51-60 (1995).
16. Rom & Kahana, Polyamines regulate the expression of ornithine decarboxylase antizyme in vitro by inducing ribosomal frame-shifting, Proc Natl Acad Sci USA. 91(9):3959-63 (1994).
17. Fages-Lartaud et al., mCherry contains a fluorescent protein isoform that interferes with its reporter function, Front Bioeng Biotechnol. 10:892138 (2022).
18. McCann & Pegg, Ornithine decarboxylase as an enzyme target for therapy, Pharmacol Ther. 54(2):195-215 (1992).
19. Dever & Ivanov, Roles of polyamines in translation. J Biol Chem. 293(48):18719-29 (2018).
20. Holbert et al., Autophagy induction by exogenous polyamines is an artifact of bovine serum amine oxidase activity in culture serum, J Biol Chem. 295(27):9061-68 (2020).
21. Ray et al., Spermidine, a sensor for antizyme 1 expression regulates intracellular polyamine homeostasis, Amino Acids 46(8):2005-13 (2014).
22. Rato et al., Translational recoding as a feedback controller: systems approaches reveal polyamine-specific effects on the antizyme ribosomal frameshift, Nucleic Acids Res. 39(11):4587-97 (2011).
23. Al-Habsi et al., Spermidine activates mitochondrial trifunctional protein and improves antitumor immunity in mice, Science 378(6618):eabj3510 (2022).
24. Marton & Pegg, Polyamines as targets for therapeutic intervention, Annu Rev Pharmacol Toxicol. 35:55-91 (1995).
25. Watanabe et al., Estimation of polyamine binding to macromolecules and ATP in bovine lymphocytes and rat liver, J Biol Chem. 266(31):20803-9 (1991).
26. Iwamoto et al., A general chemical method to regulate protein stability in the mammalian central nervous system, Chem Biol. 17(9):981-88 (2010).
27. Zhang et al., Quantitative assessment of near-infrared fluorescent proteins, Nat Methods 20(10):1605-16 (2023).
28. Stewart et al., Difluoromethylornithine rebalances aberrant polyamine ratios in Snyder-Robinson syndrome, EMBO Mol Med. 15(11):e17833 (2023).
29. Azfar et al., P5B-ATPases in the mammalian polyamine transport system and their role in disease, Biochim Biophys Acta Mol Cell Res. 1869(12):119354 (2022).
30. Eom et al., SLC3A2 and SLC7A2 Mediate the Exogenous Putrescine-Induced Adipocyte Differentiation, Mol Cells 45(12):963-75 (2022).
31. Daigle et al., Molecular characterization of a human cation-Cl− cotransporter (SLC12A8A, CCC9A) that promotes polyamine and amino acid transport, J Cell Physiol. 220(3):680-89 (2009).
32. Aouida et al., The human carnitine transporter SLC22A16 mediates high affinity uptake of the anticancer polyamine analogue bleomycin-A5, J Biol Chem. 285(9):6275-84 (2010).
33. Uemura et al., Polyamine transport is mediated by both endocytic and solute carrier transport mechanisms in the gastrointestinal tract, Am J Physiol Gastrointest Liver Physiol. 299(2):G517-22 (2010).
34. Belting et al., Glypican-1 is a vehicle for polyamine uptake in mammalian cells: a pivital role for nitrosothiol-derived nitric oxide, J Biol Chem. 278(47):47181-89 (2003).
35. Burns et al., Lipophilic lysine-spermine conjugates are potent polyamine transport inhibitors for use in combination with a polyamine biosynthesis inhibitor, J Med Chem. 52(7):1983-93 (2009).
36. Inglis et al., Coupled protein quality control during nonsense-mediated mRNA decay, J Cell Sci. 136(10):jcs261216 (2023).
37. Keren-Paz et al., Overexpression of antizyme-inhibitor in NIH3T3 fibroblasts provides growth advantage through neutralization of antizyme functions, Oncogene 25(37):5163-72 (2006).
38. Kaouass et al., The spermidine transport system is regulated by ligand inactivation, endocytosis, and by the Npr1p Ser/Thr protein kinase in Saccharomyces cerevisiae, J Biol Chem. 273(4):2109-17 (1998).
39. Ramirez et al., Hereditary parkinsonism with dementia is caused by mutations in ATP13A2, encoding a lysosomal type 5 P-type ATPase, Nat Genet. 38(10):1184-91 (2006).
40. Lin et al., Novel ATP13A2 variant associated with Parkinson disease in Taiwan and Singapore, Neurology 71(21):1727-32 (2008).
41. Spataro et al., Mutations in ATP13A2 (PARK9) are associated with an amyotrophic lateral sclerosis-like phenotype, implicating this locus in further phenotypic expansion, Hum Genomics 13(1):19 (2019).
42. Gräf et al., Identification of rare sequence variation underlying heritable pulmonary arterial hypertension, Nat Commun. 9(1):1416 (2018).
43. Burns, Intratumoral Extracellular Metabolic Impact of DFMO and AMXT 1501, clinicaltrials.gov/study/NCT05717153 (2023).
44. Gitto et al., Difluoromethylornithine Combined with a Polyamine Transport Inhibitor Is Effective against Gemcitabine Resistant Pancreatic Cancer, Mol Pharm. 15(2):369-76 (2018).
45. Williams et al., VPS35, the Retromer Complex and Parkinson's Disease, J Parkinsons Dis. 7(2):219-33 (2017).
46. Gonzilez-Rodriguez et al., Disruption of mitochondrial complex I induces progressive parkinsonism, Nature 599(7886):650-56 (2021).
47. Small & Petsko, Retromer in Alzheimer disease, Parkinson disease and other neurological disorders, Nat Rev Neurosci. 16(3):126-32 (2015).
48. Tanner et al., Rotenone, paraquat, and Parkinson's disease, Environ Health Perspect 119(6):866-72 (2011).
49. Puleston et al., Polyamines and eIF5A Hypusination Modulate Mitochondrial Respiration and Macrophage Activation, Cell Metab. 30(2):352-63.e8 (2019).
50. Birsoy et al., An Essential Role of the Mitochondrial Electron Transport Chain in Cell Proliferation Is to Enable Aspartate Synthesis, Cell 162(3):540-51 (2015).
51. Sullivan et al., Supporting Aspartate Biosynthesis Is an Essential Function of Respiration in Proliferating Cells, Cell 162(3):552-63 (2015).
52. Krall et al., Asparagine couples mitochondrial respiration to ATF4 activity and tumor growth, Cell Metab. 33(5):1013-26.e6 (2021).
53. Bao et al., Mitochondrial dysfunction remodels one-carbon metabolism in human cells, Elife 5:e10575 (2016).
54. Wu et al., Electron transport chain inhibition increases cellular dependence on purine transport and salvage, Cell Metab. 36(7):1504-20 (2024).
55. Ghandi et al., Next-generation characterization of the Cancer Cell Line Encyclopedia, Nature 569(7757):503-08 (2019).
56. Banos-Mateos et al., VPS29, a tweak tool of endosomal recycling, Curr Opin Cell Biol. 59:81-87 (2019).
57. Cho et al., OpenCell: Endogenous tagging for the cartography of human cellular organization, Science 375(6585):eabi6983 (2022).
58. Sekhar et al., ATP13A3 facilitates polyamine transport in human pancreatic cancer cells, Sci Rep. 12(1):4045 (2022).
59. Luirink & Sinning, SRP-mediated protein targeting: structure and function revisited, Biochim Biophys Acta. 1694(1-3):17-35 (2004).
60. Kellogg et al., Signal Recognition Particle in Human Diseases, Front Genet. 13:898083 (2022).
61. Karamyshev et al., Inefficient SRP interaction with a nascent chain triggers a mRNA quality control pathway, Cell 156(1-2):146-57 (2014).
62. Gupta et al., Restoring polyamines protects from age-induced memory impairment in an autophagy-dependent manner, Nat Neurosci. 16(10):1453-60 (2013).
63. Eisenberg et al., Cardioprotection and lifespan extension by the natural polyamine spermidine, Nat Med. 22(12):1428-38 (2016).
64. Zhang et al., Polyamine metabolite spermidine rejuvenates oocyte quality by enhancing mitophagy during female reproductive aging, Nat Aging 3(11):1372-86 (2023).
65. Eisenberg et al., Induction of autophagy by spermidine promotes longevity, Nat Cell Biol. 11(11):1305-14 (2009).
66. Perez-Leal et al., Polyamine-regulated translation of spermidine/spermine-N1-acetyltransferase, Mol Cell Biol. 32(8):1453-67 (2012).
67. Coffino, Regulation of cellular polyamines by antizyme, Nat Rev Mol Cell Biol. 2(3):188-94 (2001).
68. Park & Wolff, Hypusine, a polyamine-derived amino acid critical for eukaryotic translation, J Biol Chem. 293(48):18710-18 (2018).
69. Hart et al., High-Resolution CRISPR Screens Reveal Fitness Genes and Genotype-Specific Cancer Liabilities, Cell 163(6):1515-26 (2015).
70. Matsufuji et al., Reading two bases twice: mammalian antizyme frameshifting in yeast, EMBO J. 15(6):1360-70 (1996).
71. Atkins et al., Ribosomal frameshifting and transcriptional slippage: From genetic steganography and cryptography to adventitious use, Nucleic Acids Res. 44(15):7007-78 (2016).
72. Mizrahi et al., Virus-Induced Changes in mRNA Secondary Structure Uncover cis-Regulatory Elements that Directly Control Gene Expression, Mol Cell 72(5):862-874.e5 (2018).
73. Gandre et al., Mitochondrial localization of antizyme is determined by context-dependent alternative utilization of two AUG initiation codons, Mitochondrion 2(4):245-56 (2003).
74. Ivanov & Atkins, Ribosomal frameshifting in decoding antizyme mRNAs from yeast and protists to humans: close to 300 cases reveal remarkable diversity despite underlying conservation, Nucleic Acids Res. 35(6):1842-58 (2007).
75. Horlbeck et al., Compact and highly active next-generation libraries for CRISPR-mediated gene repression and activation, Elife 5:e19760 (2016).
76. Mihaylova et al., Fasting Activates Fatty Acid Oxidation to Enhance Intestinal Stem Cell Function during Homeostasis and Aging, Cell Stem Cell 22(5):769-78 (2018).
77. Sato et al., Single Lgr5 stem cells build crypt-villus structures in vitro without a mesenchymal niche, Nature 459(7244):262-5 (2009).
78. Yilmaz et al., mTORC1 in the Paneth cell niche couples intestinal stem-cell function to calorie intake, Nature 486(7404):490-95 (2012).
79. Stringer et al., Cellpose: a generalist algorithm for cellular segmentation, Nat Methods 18(1):100-106 (2021).
80. Ershov et al., TrackMate 7: integrating state-of-the-art segmentation algorithms into tracking pipelines, Nat Methods 19(7):829-32 (2022).
81. Dobin et al., STAR: ultrafast universal RNA-seq aligner, Bioinformatics 29(1):15-21 (2013).
82. Garrido-Martin et al., ggsashimi: Sashimi plot revised for browser- and annotation-independent splicing visualization, PLoS Comput Biol. 14(8):e1006360 (2018).
83. Stewart et al., Lentivirus-delivered stable gene silencing by RNAi in primary cells, RNA. 9(4):493-501 (2003).
84. Langmead et al., Ultrafast and memory-efficient alignment of short DNA sequences to the human genome, Genome Biol. 10(3):R25 (2009).
85. Li et al., MAGeCK enables robust identification of essential genes from genome-scale CRISPR/Cas9 knockout screens, Genome Biol. 15(12):554 (2014).
86. Subramanian et al., Gene set enrichment analysis: a knowledge-based approach for interpreting genome-wide expression profiles, Proc Natl Acad Sci USA. 102(43):15545-50 (2005).
87. Agrawal et al., WikiPathways 2024: next generation pathway database, Nucleic Acids Res. 52(D1):D679-D689 (2024).
88. Wei & Simko, cran.r-project.org/web/packages/corrplot/citation.html, R package ‘corrplot’: Visualization of a Correlation Matrix. (Version 0.92) (github.com/taiyun/corrplot) (2021).

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

1-2. (canceled)

3. A polynucleotide comprising, in 5′ to 3′ order:
a) a control protein coding sequence encoding a control protein,
b) a polyamine-responsive frameshifting element, and
c) a reporter protein coding sequence encoding a reporter protein, wherein:
i) the reporter protein coding sequence lacks a start codon, and
ii) expression of the reporter protein requires a polyamine-responsive frameshift.

4. The polynucleotide of claim 3, wherein the reporter protein coding sequence and the control protein coding sequence are out of frame with each other.

5. The polynucleotide of claim 3, wherein the reporter protein coding sequence is in the +1-frame of the control protein coding sequence.

6. The polynucleotide of claim 3, comprising, in 5′ to 3′ order:
a) the control protein coding sequence,
b) a stop codon, wherein the control protein coding sequence and the stop codon are in the same contiguous open reading frame,
c) the polyamine-responsive frameshifting element, and
d) the reporter protein coding sequence.

7. The polynucleotide of claim 3, wherein the polyamine-responsive frameshift is a +1 ribosomal frameshift.

8. The polynucleotide of claim 3, wherein the polyamine-responsive frameshifting element is derived from an ornithine decarboxylase antizyme 1 gene.

9. The polynucleotide of claim 3, wherein the polyamine-responsive frameshifting element is derived from a human ornithine decarboxylase antizyme 1 (OAZ1) gene.

10. The polynucleotide of claim 3, wherein the polyamine-responsive frameshifting element lacks a catalytically active domain.

11. The polynucleotide of claim 3, wherein the polyamine-responsive frameshifting element comprises:
TCCTGA
and
(SEQ ID NO: 2)
TGCCCCTCACCCACCCCTGAAGATCCCAGGTGGGCGAGGGAATAGTCA
GAGGGATCACAA;
(SEQ ID NO: 3)
TCCTGATGCCCCTCACCCACCCCTGAAGATCCCAGGTGGGCGAGGGAA
TAGTCAGAGGGATCACAA;
(SEQ ID NO: 4)
CGGGGCCTCGGTGGTGCTCCTGATGCCCCTCACCCACCCCTGAAGATC
CCAGGTGGGCGAGGGAATAGTCAGAGGGATCACAATCTTTCAG;
(SEQ ID NO: 5)
CTCCTAAGCCTGCACAGCCGCGGCGGCAGCAGCAGTGAGAGTTCCAGG
GTCTCCCTCCACTGCTGTAGTAACCCGGGTCCGGGGCCTCGGTGGTGC
TCCTGATGCCCCTCACCCACCCCTGAAGATCCCAGGTGGGCGAGGGAA
TAGTCAGAGGGATCACAATCTTTCAG,
or a combination of the foregoing.

12-14. (canceled)

15. The polynucleotide of claim 3, wherein the reporter protein comprises a fluorescent protein.

16-18. (canceled)

19. A polynucleotide, comprising, in 5′ to 3′ order:
an mCherry coding sequence encoding an mCherry,
a polyamine-responsive frameshifting element derived from a human OAZ1 gene, and
a reporter protein coding sequence encoding an eYFP or miRFP670-2, wherein the reporter protein coding sequence lacks an in-frame start codon,
wherein interaction between a polyamine and the polyamine-responsive frameshifting element increases expression of the eYFP or miRFP670-2.

20. A polynucleotide, comprising, in 5′ to 3′ order:
an mCherry coding sequence encoding an mCherry,
a stop codon,
a polyamine-responsive frameshifting element derived from a human OAZ1 gene, and
a reporter protein coding sequence encoding an eYFP or miRFP670-2,
wherein interaction between a polyamine and the polyamine-responsive frameshifting element increases expression of the eYFP or miRFP670-2, and wherein
a) the reporter protein coding sequence lacks an in-frame start codon,
b) the mCherry coding sequence has an in-frame start codon, or
both a) and b).

21. The polynucleotide of claim 3, wherein the reporter protein coding sequence is fused to a protein-degradation tag coding sequence, a nuclear localization signal, or both.

22-27. (canceled)

28. A vector comprising the polynucleotide of claim 3.

29. A cell comprising the polynucleotide of claim 3 or a vector comprising the polynucleotide of claim 3.

30-38. (canceled)

39. A kit comprising the polynucleotide of claim 3, a vector comprising the polynucleotide of claim 3, or a cell comprising the polynucleotide of claim 3 or the vector.

40. A method of reporting an intracellular polyamine, comprising providing the cell of claim 29, and obtaining a measurement comprising:
a) an expression of the reporter protein in the cell, or a temporal change thereof,
b) a ratio of expression between the reporter protein and the control protein, or a temporal change thereof,
or both a) and b).

41. The method of claim 40, wherein the method provides a quantitative readout of the level of the intracellular polyamine.

42-44. (canceled)

45. The method of claim 40, wherein the method reports intracellular spermidine, spermine, or both.

46. The method of claim 40, wherein the method reports intracellular spermidine.

47-51. (canceled)


SEQUENCE LISTING (26 sequences)

SEQ ID NO: 1 - RNA, 1195 nt, Homo sapiens
  source 1..1195: mol_type = mRNA
  misc_feature 1..1195: note = ornithine decarboxylase antizyme 1
agcatctataaaggcgggcggcggcagaggcgccattttgcgaacggcgagcagcggcggcggcgcggagagacgcagcggaggttttcctggtttcggaccccagcggccggatggtgaaatcctccctgcagcggatcctcaatagccactgcttcgccagagagaaggaaggggataaacccagcgccaccatccacgccagccgcaccatgccgctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcagctaacttattctactccgatgatcggctgaatgtaacagaggaactaacgtccaacgacaagacgaggattctcaacgtccagtccaggctcacagacgccaaacgcattaactggcgaacagtgctgagtggcggcagcctctacatcgagatcccgggcggcgcgctgcccgaggggagcaaggacagctttgcagttctcctggagttcgctgaggagcagctgcgagccgaccatgtcttcatttgcttccacaagaaccgcgaggacagagccgccttgctccgaaccttcagctttttgggctttgagattgtgagaccggggcatccccttgtccccaagagacccgacgcttgcttcatggcctacacgttcgagagagagtcttcgggagaggaggaggagtagggccgcctcggggctgggcatccggcccctggggccaccccttgtcagccgggtgggtaggaaccgtagactcgctcatctcgcctgggtttgtccgcatgttgtaatcgtgcaaataaacgctcactccgaattagcggtgtatttcttgaagtttaatattgtgtttgtgatactgaagtatttgctttaattctaaataaaaatttatattttacttttttattgctggtttaagatgattcagattatccttgtactttgaggagaagtttcttatttggagtcttttggaaacagtcttagtcttttaacttggaaagatgaggtattaatcccctccattgctctccaaaagccaataaagtgattacacccgaaaaaaaaaaaaaaa

SEQ ID NO: 2 - RNA, 60 nt, Homo sapiens
  source 1..60: mol_type = mRNA
tgcccctcacccacccctgaagatcccaggtgggcgagggaatagtcagagggatcacaa

SEQ ID NO: 3 - RNA, 66 nt, Homo sapiens
  source 1..66: mol_type = mRNA
tcctgatgcccctcacccacccctgaagatcccaggtgggcgagggaatagtcagagggatcacaa

SEQ ID NO: 4 - RNA, 91 nt, Homo sapiens
  source 1..91: mol_type = mRNA
cggggcctcggtggtgctcctgatgcccctcacccacccctgaagatcccaggtgggcgagggaatagtcagagggatcacaatctttcag

SEQ ID NO: 5 - RNA, 170 nt, Homo sapiens
  source 1..170: mol_type = mRNA
ctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcag

SEQ ID NO: 6 - RNA, 275 nt, Homo sapiens
  source 1..275: mol_type = mRNA
atggtgaaatcctccctgcagcggatcctcaatagccactgcttcgccagagagaaggaaggggataaacccagcgccaccatccacgccagccgcaccatgccgctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcag

SEQ ID NO: 7 - DNA, 12 bp, synthetic construct
  source 1..12: mol_type = other DNA
ggctccggaggt

SEQ ID NO: 8 - RNA, 20 nt, synthetic construct
  source 1..20: mol_type = other RNA
  misc_feature 1..20: note = ODC1 sgRNA
gggcggcggcggctacagga

SEQ ID NO: 9 - RNA, 20 nt, synthetic construct
  source 1..20: mol_type = other RNA
  misc_feature 1..20: note = SRM sgRNA
ggtagcgcgagcgccggtgg

SEQ ID NO: 10 - RNA, 20 nt, synthetic construct
  source 1..20: mol_type = other RNA
  misc_feature 1..20: note = ATP13A3 sgRNA
tgggtgaaataacgaatctg

SEQ ID NO: 11 - DNA, 48 bp, synthetic construct
  source 1..48: mol_type = other DNA
  misc_feature 1..48: note = forward primer
aatgatacggcgaccaccgagatctacaccccactgacgggcaccgga

SEQ ID NO: 12 - DNA, 54 bp, synthetic construct
  source 1..54: mol_type = other DNA
  misc_feature 1..54: note = reverse primer
caagcagaagacggcatacgagatcnnnnnntttcttgggtagtttgcagtttt

SEQ ID NO: 13 - DNA, 60 bp, synthetic construct
  source 1..60: mol_type = other DNA
  misc_feature 1..60: note = read 1 sequencing primer
gttgataacggactagccttatttaaacttgctatgctgtttccagcatagctcttaaac

SEQ ID NO: 14 - DNA, 71 bp, synthetic construct
  source 1..71: mol_type = other DNA
  misc_feature 1..71: note = index sequencing primer
tttcaagttacggtaagcatatgatagtccattttaaaacataattttaaaactgcaaactacccaagaaa

SEQ ID NO: 15 - DNA, 10423 bp, synthetic construct
  source 1..10423: mol_type = other DNA
  misc_feature 1..10423: note = reporter construct
cgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtctttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtgaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattaaataataaactagtctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcaggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtgatgaattgcggccgcctgacgtaactagtgtaaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 16 - DNA, 10417 bp, synthetic construct
  source 1..10417: mol_type = other DNA
  misc_feature 1..10417: note = reporter construct (pHR-Tre3G-mCherry-OAZ1 FS-eYFP)
cgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtctttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtgaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattaacactagtctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcaggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtgatgaattgcggccgcctgacgtaactagtgtaaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 17 - DNA, 14904 bp, synthetic construct
  source 1..14904: mol_type = other DNA
  misc_feature 1..14904: note = ornithine decarboxylase knockdown (pLenti hU6-ODC1_sgRNA hUbC-dCas9-KRAB-T2A-Puro)
cgccataactgccgcccccgcaacagacgacaaaccgagttctccagtcagtgacaaacttcacgtcagggtccccagatggtgccccagcccatctcacccgaataagagctttcccgcattagcgaaggcctcaagaccttgggttcttgccgcccaccatgccccccaccttgtttcaacgacctcacagcccgcctcacaagcgtcttccattcaagactcgggaacagccgccattttgctgcgctccccccaacccccagttcagggcaaccttgctcgcggacccagactacagcccttggcggtctctccacacgcttccgtcccaccgagcggcccggcggccacgaaagccccggccagcccagcagcccgctactcaccaagtgacgatcacagcgatccacaaacaagaaccgcgacccaaatcccggctgcgacggaactagctgtgccacacccggcgcgtccttatataatcatcggcgttcaccgccccacggagatccctccgcagaatcgccgagaagggactacttttcctcgcctgttccgctctctggaaagaaaaccagtgccctagagtcacccaagtcccgtcctaaaatgtccttctgctgatactggggttctaaggccgagtcttatgagcagcgggccgctgtcctgagcgtccgggcggaaggatcaggacgctcgctgcgcccttcgtctgacgtggcagcgctcgccgtgaggaggggggcgcccgcgggaggcgccaaaacccggcgcggaggccagatctggagccgacacgggttaattaattaagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggcggcggcggctacaggagttttagagctagaaatagcaagttaaaataaggctagtccgttatcaacttgaaaaagtggcaccgagtcggtgctttttttttaataacttcgtataatgtatgctatacgaagttattaattaaccaaactggatctctgctgtccctgtaataaacccgaaaattttgaatttttgtaatttgtttttgtaattctttagtttgtatgtctgttgctattatgtctactattctttcccctgcactgtaccccccaatccccccttttcttttaaaattgtggatgaatactgccatttgtctgcagaattggcgcacgcagtgccgatccgttcactaatcgaatggatctgtctctgtctctctctccaccttcttcttctattccttcgggcctgtcgggtcccctcggggttgggaggtgggtctgaaacgataatggtgaatatccctgcctaactctattcactatagaaagtacagcaaaaactattcttaaacctaccaagcctcctactatcattatgaataattttatataccacagccaatttgttatgttaaaccaattccacaaacttgcccatttatctaattccaataattcttgttcattcttttcttgctggttttgcgattcttcaattaaggagtgtattaagcttgtgtaattgttaatttctctgtcccactccatccaggtcgtgtgattccaaatctgttccagagatttattactccaactagcattccaaggcacagcagtggtgcaaatgagttttccagagcaaccccaaatccccaggagctgttgatcctttaggtatctttccacagccaggattcttgcctggagctgcttgatgccccagactgtgagttgcaacagatgctgttgcgcctcaatagccctcagcaaattgttctgctgctgcactataccagacaataattgtctggcctgtaccgtcagcgtcattgacgctgcgcccatagtgcttcctgctgctcccaagaacccaaggaacaaagctcctattcccactgctcttttttctctctgcaccactcttctctttgccttggtgggtgctactcctaatggttcaatttttactactttatatttatataattcacttctccaattgtccctcatatctcctcctccaggtctgaagatcagcggccgcttgctgtgcggtggtcttacttttgttttgctcttcctctatcttgtctaaagcttccttggtgtcttttatctctatcctttgatgcacacaatagagggttgctactgtattatataatgatctaagttcttctgatcctgtctgaagggatggttgtagctgtcccagtatttgtctacagccttctgatgtttctaacaggccaggattaactgcgaatcgttctagctccctgcttgcccatactatatgttttaatttatattttttctttccccctggccttaaccgaattttttcccatcgcgatctaattctcccccgcttaatactgacgctctcgcacccatctctctccttctagcctccgctagtcaaaatttttggcgtactcaccagtcgccgcccctcgcctcttgccgtgcgcgcttcagcaagccgagtcctgcgtcgagagagctcctctggtttccctttcgctttcaagtccctgttcgggcgccactgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacaggcaaaacgcgctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcaacgcgtatatctggcccgtacatcgcgaagcagcgcaaaacgcctaaccctaagcagattcttcatgcaattgtcggtcaagccttgccttgttgtagcttaaattttgctcgcgcactactcagcgacctccaacacacaagcagggagcagatactggcttaactatgcggcatcagagcagattgtactgagagtgcaccataggggatcgggagatctcccgatccgtcgacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctctagctagaggtcgacggtatacagacatgataagatacattgatgagtttggacaaaccacaactagaatgcagtgaaaaaaatgctttatttgtgaaatttgtgatgctattgctttatttgtaaccattataagctgcaataaacaagttggggtgggcgaagaactccagcatgagatccccgcgctggaggatcatccagccggcgtcccggaaaacgattccgaagcccaacctttcatagaaggcggcggtggaatcgaaatctcgtagcacgtgtcagtcctgctcctcggccacgaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatggtttagttcctcaccttgtcgtattatactatgccgatatactatgccgatgattaattgtcaacacgtgctgatcagatccgaaaatggatatacaagctcccgggagctttttgcaaaagcctaggcctccaaaaaagcctcctcactacttctggaatagctcagaggcagaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagaattaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtggggataccccctagagccccagctggttctttccgcctcagaagccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagcgggtttaaacgggccctgctagagattttccacactgactaaaagggtctgagggatctctagttaccagagtcacacaacagacgggcacacactacttgaagcactcaaggcaagctttattgaggcttaagcagtgggttccctagttagccagagagctcccaggctcagatctggtctaaccagagagacccagtacagtccggatgcagctctcgggccatgtgatgaaatgctaggcggctgtcaaacctccactctaatacttctctctccgggtcatccatcccatgcaggctcacagggtgtaacaagcgggtgttctctccttcattggcttcttctaccttctcttgctcaactggtactagcttgtagcaccatccaaaggtcagtggatatctgatccctggccctggtgtgtagttctgccaatcagggaagtagccttgtgtgtggtagatccacagatcaaggatatcttgtcttcgttgggagtgaattagcccttccagtccccccttttcttttaaaaagtggctaagatctacagctgccttgtaagtcattggtcttaaaggtacctgaggtgtgactggaaaacccacctcctcctcctcttgtgcttctagccaggcacaatcagcattggtagctgctgtattgctacttgtgattgctccatgtttttctaggtctcgaggtcgacggtatcgatgcggggaggcggcccaaagggagatccgactcgtctgagggcgaaggcgaagacgcggaagaggccgcagagccggcagcaggccgcgggaaggaaggtccgctggattgagggccgaagggacgtagcagaaggacgtcccgcgcagaatccaggtggcaacacaggcgagcagccaaggaaaggacgatgatttccccgacaacaccacggaattgtcagtgcccaacagccgagcccctgtccagcagcgggcaaggcaggcggcgatgagttccgccgtggcaatagggagggggaaagcgaaagtcccggaaaggagctgacaggtggtggcaatgccccaaccagtgggggttgcgtcagcaaacacagtgcacaccacgccacgttgcctgacaacgggccacaactcctcataaagagacagcaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatcgataagcttgatatcgaattcataacttcgtataatgtatgctatacgaagttatctattatcaaccggtcgtgctgcagaagctagtaacggccgccagtgtgctggtcaggcaccgggcttgcgggtcatgcaccaggtgcgcggtccttcgggcacctcgacgtcggcggtgacggtgaagccgagccgctcgtagaaggggaggttgcggggcgcggaggtctccaggaaggcgggcaccccggcgcgctcggccgcctccactccggggagcacgacggcgctgcccagacccttgccctggtggtcgggcgacacgccgacggtggccaggaaccacgcgggctccttgggccggtgcggcgccaggaggccttccatctgttgctgcgcggccagccgggaaccgctcaactcggccatgcgcgggccgatctcggcgaacaccgcccccgcttcgacgctctccggcgtggtccagaccgccaccgcggcgccgtcgtccgcgacccacaccttgccgatgtcgagcccgacgcgcgtgaggaagagttcttgcagctcggtgacccgctcgatgtggcggtccggatcgacggtgtggcgcgtggcggggtagtcggcgaacgcggcggcgagggtgcgtacggccctggggacgtcgtcgcgggtggcgaggcgcaccgtgggcttgtactcggtcatagggccgggattctcctccacgtcaccgcatgttagaagacttcctctgccctcgctagcaactttgcgtttctttttcggaactgatgatttgatttcaaatgcagtctctgaatcaggatgggtctcttggtgaatttctctctccaccagccagggctcttctcccttctccaaccggaggatcacatctggcttagtaagctgataacccaaggaaaccaggttcttatagttctccagcatcacatttctgtacaggatctgctgagcagtgtccagcagcttccactcctccctggtgaagtccacaaacacatccttgaaggtcaccagtgtccgggaccaggcagtcagtgacttagcatcgctagccaccttcctcttcttcttggggtcagccctgctgtctccaccgagctgagagaggtcgattcttgtttcatagagccccgtaattgactgatgaatcagtgtggcgtccaggacctcctttgtagaggtgtaccgctttctgtctatggtggtgtcgaagtacttgaaggctgcaggcgcgcccaagttggtcagagtaaacaagtggataatgttttctgcctgctccctgatgggcttatccctgtgcttattgtaagcagaaagcaccttatcgaggttagcgtcggcgaggatcactcttttggagaattcgcttatttgctcgatgatctcatcaaggtagtgtttgtgttgttccacgaacagctgcttctgctcattatcttcgggagaccctttgagcttttcatagtggctggccagatacaagaaattaacgtatttagagggcagtgccagctcgttacctttctgcagctcgcccgcactagcgagcattcgtttccggccgttttcaagctcaaagagagagtacttgggaagcttaatgatgaggtcttttttgacctctttatatcctttcgcctcgagaaagtcgatggggtttttttcgaagcttgatcgctccatgattgtgatgcccagcagttccttgacgcttttgagttttttagacttccctttctccactttggccacaaccagtacactgtaagcgactgtaggagaatcgaatccgccgtatttcttggggtcccaatcttttttgcgtgcgatcagcttgtcgctgttccttttcgggaggatactttccttggagaagcctccggtctgtacttcggtctttttaacgatgttcacctgcggcatggacaggaccttccggactgtcgcgaaatccctacccttgtcccacacgatttctcctgtttctccgtttgtttcgataagtggtcgcttccgaatctctccattggccagtgtaatctcggtcttgaaaaaattcataatattgctgtaaaagaagtacttagcggtggccttgcctatttcctgctcagactttgcgatcattttcctaacatcgtacactttatagtctccgtaaacaaattcagattcaagcttgggatattttttgataagtgcagtgcctaccactgcattcaggtaggcatcatgcgcatggtggtaattgttgatctctctcaccttataaaactgaaagtcctttctgaaatctgagaccagcttagacttcagagtaataactttcacctctcgaatcagtttgtcattttcatcgtacttggtgttcatgcgtgaatcgagaatttgggccacgtgcttggtgatctggcgtgtctcaacaagctgccttttgatgaagccggctttatccaactcagacaggccacctcgttcagccttagtcagattatcgaacttccgttgtgtgatcagtttggcgttcagcagctgccgccaataatttttcattttcttgacaacttcttctgaggggacgttatcactcttccctctatttttatcggatcttgtcaacactttattatcaatagaatcatctttgagaaaagactggggcacgatggcatccacgtcgtagtcggagagccgattgatgtccagttcctgatccacgtacatgtccctgccgttctgcaggtagtacaggtagagcttctcattctgaagctgggtgttttcaactgggtgttccttaaggatttgggaccccagttcttttataccctcttcaatcctcttcatcctttccctactgttcttctgtcccttctgggtagtttggttctctcgggccatctcgataacgatattctcgggcttatgccttcccattactttgacgagttcatccacgaccttaacggtctgcagtattccctttttgatagctgggctacctgcaagattagcgatgtgctcgtgaagactgtccccctggccagaaacttgtgctttctggatgtcctccttaaaggtgagagagtcatcatggatcaactgcatgaagttccggttggcaaatccatcggacttaagaaaatccaggattgtctttccactctgcttgtctcggatcccattgatcagttttcttgacagccgcccccatcctgtatatcggcgcctcttgagctgtttcatgactttgtcgtcgaagagatgagcgtaagttttcaagcgttcttcaatcatctccctatcttcaaacaacgtaagggtgaggacaatgtcctcaagaatgtcctcgttctcctcattgtccaggaagtccttgtctttaatgattttcaggagatcgtgatacgttcccagggatgcgttgaagcgatcctccactccgctgatttcaacagagtcgaaacattcaatctttttgaaatagtcttctttgagctgtttcacggtaactttccggttcgtcttgaagaggaggtccacgatagctttcttctgctctccagacaggaatgctggctttctcatcccttctgtgacgtatttgaccttggtgagctcgttataaactgtgaagtactcgtacagcagagagtgtttaggaagcaccttttcgttaggcagatttttatcaaagttagtcatcctttcgatgaaggactgggcagaggcccccttatccacgacttcctcgaagttccagggagtgatggtctcttctgatttgcgagtcatccacgcgaatctggaatttccccgggcgagggggcctacatagtagggtatccgaaatgtgaggattttctcaatcttttccctgttatctttcaaaaaggggtagaaatcctcttgccgcctgaggatagcgtgcagttcgcccaggtgaatctggtgggggatgcttccattgtcgaaagtgcgctgtttgcgcaacagatcttctctgttaagctttaccagcagctcctcggtgccgtccattttttccaagatgggcttaataaatttgtaaaattcctcctggcttgctccgccgtcaatgtatccggcgtagccatttttagactgatcgaagaaaatttccttgtacttctcaggcagttgctgtctgacaagggccttcagcaaagtcaagtcttggtggtgctcatcatagcgcttgatcatactagcgctcagcggagctttggtgatctccgtgttcactcgcagaatatcactcagcagaatggcgtctgacaggttctttgccgccaaaaaaaggtctgcgtactggtcgccgatctgggccagcagattgtcgagatcatcatcgtaggtgtctttgctcagttgaagcttggcatcttcggccaggtcgaagttagatttaaagttgggggtcagcccgagtgacagggcgataagattaccaaacaggccgttcttcttctccccagggagctgtgcgatgaggttttcgagccgccgggatttggacagcctagcgctcaggattgctttggcgtcaactccggatgcgttgatcgggttctcttcgaaaagctgattgtaagtctgaaccagttggataaagagtttgtcgacatcgctgttgtctgggttcaggtccccctcgatgaggaagtgtccccgaaatttgatcatatgcgccagcgcgagatagatcaaccgcaagtcagccttatcagtactgtctacaagcttcttcctcagatgatatatggttgggtacttttcatggtacgccacctcgtccacgatattgccaaagattgggtggcgctcgtgctttttatcctcctccaccaaaaaggactcctccagcctatggaagaaagagtcatccaccttagccatctcattactaaagatctcctgcaggtagcagatccgattctttctgcgggtatatctgcgccgtgctgttcttttgagccgcgtggcttcggcggtttccccggagtcgaacaggagggcgccaatgaggttcttctttatgctgtggcgatcggtattgcccagaactttgaattttttgctcggcaccttgtactcgtccgtaatgacggcccagccgacgctgtttgtgccgatggcgagcccaatggagtacttcttgtccattccgcggcccaccttcctcttcttcttgggggccatcttgtcatcgtcatccttgtaatcgatgtcatgatctttataatcaccgtcatggtctttgtagtccatggtggctctagagtcgacctgcagcccaagcttcgtctaacaaaaaagccaaaaacggccagaatttagcggacaatttactagtctaacactgaaaattacatattgacccaaatgattacatttcaaaaggtgcctaaaaaacttcacaaaacacactcgccaaccccgagcgcatagttcaaaaccggagcttcagctacttaagaagataggtacataaaaccgaccaaagaaactgacgcctcacttatccctcccctcaccagaggtccggcgcctgtcgattcaggagagcctaccctaggcccgaaccctgcgtcctgcgacggagaaaagcctaccgcacacctaccggcaggtggccccaccctgcattataagccaacagaacgggtgacgtcacgacacgacgagggcgcgcgctcccaaaggtacgggtgcactgcccaacggcac

SEQ ID NO: 18 - DNA, 14904 bp, synthetic construct
  source 1..14904: mol_type = other DNA
  misc_feature 1..14904: note = spermidine synthase knockdown (pLenti hU6-SRM_sgRNA hUbC-dCas9-KRAB-T2A-Puro)
gtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgaagcttgggctgcaggtcgactctagagccaccatggactacaaagaccatgacggtgattataaagatcatgacatcgattacaaggatgacgatgacaagatggcccccaagaagaagaggaaggtgggccgcggaatggacaagaagtactccattgggctcgccatcggcacaaacagcgtcggctgggccgtcattacggacgagtacaaggtgccgagcaaaaaattcaaagttctgggcaataccgatcgccacagcataaagaagaacctcattggcgccctcctgttcgactccggggaaaccgccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatgccatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtggctagcgatgctaagtcactgactgcctggtcccggacactggtgaccttcaaggatgtgtttgtggacttcaccagggaggagtggaagctgctggacactgctcagcagatcctgtacagaaatgtgatgctggagaactataagaacctggtttccttgggttatcagcttactaagccagatgtgatcctccggttggagaagggagaagagccctggctggtggagagagaaattcaccaagagacccatcctgattcagagactgcatttgaaatcaaatcatcagttccgaaaaagaaacgcaaagttgctagcgagggcagaggaagtcttctaacatgcggtgacgtggaggagaatcccggccctatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtgtcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaccagcacactggcggccgttactagcttctgcagcacgaccggttgataatagataacttcgtatagcatacattatacgaagttatgaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagacctagaaaaacatggagcaatcacaagtagcaatacagcagctaccaatgctgattgtgcctggctagaagcacaagaggaggaggaggtgggttttccagtcacacctcaggtacctttaagaccaatgacttacaaggcagctgtagatcttagccactttttaaaagaaaaggggggactggaagggctaattcactcccaacgaagacaagatatccttgatctgtggatctaccacacacaaggctacttccctgattggcagaactacacaccagggccagggatcagatatccactgacctttggatggtgctacaagctagtaccagttgagcaagagaaggtagaagaagccaatgaaggagagaacacccgcttgttacaccctgtgagcctgcatgggatggatgacccggagagagaagtattagagtggaggtttgacagccgcctagcatttcatcacatggcccgagagctgcatccggactgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaataacttcgtatagcatacattatacgaagttattaaaaaaaaagcaccgactcggtgccactttttcaagttgataacggactagccttattttaacttgctatttctagctctaaaacccaccggcgctcgcgctaccggtgtttcgtcctttccacaagatatataaagccaagaaatcgaaatactttcaagttacggtaagcatatgatagtccattttaaaacataattttaaaactgcaaactacccaagaaattattactttctacgtcacgtattttgtactaatatctttgtgtttacagtcaaattaattccaattatctctctaacagccttgtatcgtatatgcaaatatgaaggaatcatgggaaataggccctcttaattaattaacccgtgtcggctccagatctggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcg

SEQ ID NO: 19 - DNA, 11339 bp, synthetic construct
  source 1..11339: mol_type = other DNA
  misc_feature 1..11339: note = temporal polyamine reporter(pLenti SFFV-DHFR-NLS-mCherry-OAZ1-miRFP670_2-WPRE)
atgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggtgatcttcagacctggacgatatatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggatcgataagcttgatatcgaattcctgcagccccgataaaataaaagattttatttagtctccagaaaaaggggggaatgaaagaccccacctgtaggtttggcaagctagctgcagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggggggatctggagctctcgagaattctcagccaccatgatcagtctgattgcggcgttagcggtagattacgttatcggcatggaaaacgccatgccgtggaacctgcctgccgatctcgcctggtttaaacgcaacaccttaaataaacccgtgattatgggccgccatacctgggaatcaatcggtcgtccgttgccaggacgcaaaaatattatcctcagcagtcaaccgagtacggacgatcgcgtaacgtgggtgaagtcggtggatgaagccatcgcggcgtgtggtgacgtaccagaaatcatggtgattggcggcggtcgcgttattgaacagttcttgccaaaagcgcaaaaactgtatctgacgcatatcgacgcagaagtggaaggcgacacccatttcccggattacgagccggatgactgggaatcggtattcagcgaattccacgatgctgatgcgcagaactctcacagctattgctttgagattctggagcggcgaggctcgagccccaagaagaagcgcaaggtgggaggaggcggaactagtgtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattaacactagtctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcaggatccgcgcgtaaggtcgatctcacctcctgcgatcgcgagccgatccacatccccggcagcattcagccgtgcggctgcctgctagcctgcgacgcgcaggcggtgcggatcacgcgcattacggaaaatgccggcgcgttctttggacgcgaaactccgcgggtcggtgagctactcgccgattacttcggcgagaccgaagcccatgcgctgcgcaacgcactggcgcagtcctccgatccaaagcgaccggcgctgatcttcggttggcgcgacggcctgaccggccgcaccttcgacatctcactgcatcgccatgacggtacatcgatcatcgagttcgagcctgcggcggccgaacaggccgacaatccgctgcggctgacgcggcagatcatcgcgcgcaccaaagaactgaagtcgctcgaagagatggccgcacgggtgccgcgctatctgcaggcgatgctcggctatcaccgcgtgatgttgtaccgcttcgcggacgacggctccgggatggtgatcggcgaggcgaagcgcagcgacctcgagagctttctcggtcagcactttccggcgtcgctggtcccgcagcaggcgcggctactgtacttgaagaacgcgatccgcgtggtctcggattcgcgcggcatcagcagccggatcgtgcccgagcacgacgcctccggcgccgcgctcgatctgtcgttcgcgcacctgcgcagcatctcgccctgccatctcgaatttctgcggaacatgggcgtcagcgcctcgatgtcgctgtcgatcatcattgacggcacgctatggggattgatcatctgtcatcattacgagccgcgtgccgtgccgatggcgcagcgcgtcgcggccaaaaggttcgccgagcgcttatcgacgcacttcaccgccgcccaccaccaacgctaagcggccgcctgacgtaactagtgtaagggcgactctagagtcgacctgcaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctagctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatg

SEQ ID NO: 20 - DNA, 10507 bp, synthetic construct
  source 1..10507: mol_type = other DNA
  misc_feature 1..10507: note = OAZ1 (1-275) frameshift reporter (pHR-Tre3G-mCherry-OAZ1 FS-eYFP)
cgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtctttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtgaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattgtgaaatcctccctgcagcggatcctcaatagccactgcttcgccagagagaaggaaggggataaacccagcgccaccatccacgccagccgcaccatgccgctcctaagcctgcacagccgcggcggcagcagcagtgagagttccagggtctccctccactgctgtagtaacccgggtccggggcctcggtggtgctcctgatgcccctcacccacccctgaagatcccaggtgggcgagggaatagtcagagggatcacaatctttcaggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtgatgaattgcggccgcctgacgtaactagtgtaaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 21 - DNA, 10327 bp, synthetic construct
  source 1..10327: mol_type = other DNA
  misc_feature 1..10327: note = OAZ1 (185-275) frameshift reporter (pHR-Tre3G-mCherry-OAZ1 FS-eYFP)
cgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtctttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtgaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattccggggcctcggtggtgctcctgatgcccctcacccacccctgaagatcccaggtgggcgagggaatagtcagagggatcacaatctttcaggcgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtgatgaattgcggccgcctgacgtaactagtgtaaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 22 - DNA, 10447 bp, synthetic construct
  source 1..10447: mol_type = other DNA
  misc_feature 1..10447: note = OAZ1 (1-216) frameshift reporter (pHR-Tre3G-mCherry-OAZ1 FS-eYFP)
cgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtctttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtgaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattgtgaaatcctccctgcagcggatcctcaatagccactgcttcgccagagagaaggaaggggataaacccagcgccaccatccacgccagccgcaccatgccgctcctaagcctgcacagccgcggcggcagcagcagtgagagttccagggtctccctccactgctgtagtaacccgggtccggggcctcggtggtgctcctgatgcccctcacgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtgatgaattgcggccgcctgacgtaactagtgtaaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 23 - DNA, 10550 bp, synthetic construct
  source 1..10550: mol_type = other DNA
  misc_feature 1..10550: note = SARS Cov2 -1 frameshift reporter (pHR-Tre3G-mCherry-SARS_Cov2 FS-eYFP)
cgataccgtcgacctcgagggaattaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagcatctagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagccagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtctttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagcttggacacaagacaggcttgcgagatatgtttgagaataccactttatcccgcgtcagggagaggcagtgcgtaaaaagacgcggactcatgtgaaatactggtttttagtgcgccagatctctataatctcgcgcaacctattttcccctcgaacactttttaagccgtagataaacaggctgggacacttcacatgagcgaaaaatacatcgtcacctgggacatgttgcagatccatgcacgtaaactcgcaagccgactgatgccttctgaacaatggaaaggcattattgccgtaagccgtggcggtctgtaccgggtgcgttactggcgcgtgaactgggtattcgtcatgtcgataccgtttgtatttccagctacgatcacgacaaccagcgcgagcttaaagtgctgaaacgcgcagaaggcgatggcgaaggcttcatcgttattgatgacctggtggataccggtggtactgcggttgcgattcgtgaaatgtatccaaaagcgcactttgtcaccatcttcgcaaaaccggctggtcgtccgctggttgatgactatgttgttgatatcccgcaagatacctggattgaacagccgtgggatatgggcgtcgtattcgtcccgccaatctccggtcgctaatcttttcaacgcctggcactgccgggcgttgttctttttaacttcaggcgggttacaatagtttccagtaagtattctggaggctgcatccatgacacaggcaaacctgagcgaaaccctgttcaaaccccgctttaaacatcctgaaacctcgacgctagtccgccgctttaatcacggcgcacaaccgcctgtgcagtcggcccttgatggtaaaaccatccctcactggtatcgcatgattaaccgtctgatgtggatctggcgcggcattgacccacgcgaaatcctcgacgtccaggcacgtattgtgatgagcgatgccgaacgtaccgacgatgatttatacgatacggtgattggctaccgtggcggcaactggatttatgagtgggccccggatctttgtgaaggaaccttacttctgtggtgtgacataattggacaaactacctacagagatttaaagctctaaggtaaatataaaatttttaagtgtataatgtgttaaactactgattctaattgtttgtgtattttagattccaacctatggaactgatgaatgggagcagtggtggaatgcctttaatgaggaaaacctgttttgctcagaagaaatgccatctagtgatgatgaggctactgctgactctcaacattctactcctccaaaaaagaagagaaaggtagaagaccccaaggactttccttcagaattgctaagttttttgagtcatgctgtgtttagtaatagaactcttgcttgctttgctatttacaccacaaaggaaaaagctgcactgctatacaagaaaattatggaaaaatattctgtaacctttataagtaggcataacagttataatcataacatactgttttttcttactccacacaggcatagagtgtctgctattaataactatgctcaaaaattgtgtacctttagctttttaatttgtaaaggggttaataaggaatatttgatgtatagtgccttgactagagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcaactggataactcaagctaaccaaaatcatcccaaacttcccaccccataccctattaccactgccaattacctagtggtttcatttactctaaacctgtgattcctctgaattattttcattttaaagaaattgtatttgttaaatatgtactacaaacttagtagttggaagggctaattcactcccaaagaagacaagatatccttgatctgtggatctaccacacacaaggctacttccctgattagcagaactacacaccagggccaggggtcagatatccactgacctttggatggtgctacaagctagtaccagttgagccagataaggtagaagaggccaataaaggagagaacaccagcttgttacaccctgtgagcctgcatgggatggatgacccggagagagaagtgttagagtggaggtttgacagccgcctagcatttcatcacgtggcccgagagctgcatccggagtacttcaagaactgctgatatcgagcttgctacaagggactttccgctggggactttccagggaggcgtggcctgggcgggactggggagtggcgagccctcagatcctgcatataagcagctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtgaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgccaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttatcgatgaggccctttcgtcttcactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtatgtcgaggtaggcgtgtacggtgggcgcctataaaagcagagctcgtttagtgaaccgtcagatcgcctggagcaattccacaacacttttgtcttatacttacgcgtgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagggctccggaggtttaattaaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgatggtggtggtggatccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccaaactgagcaaagaccccaacgagaagcgcgatcacatggtcctgctggagttcgtgaccgccgccgggatcactctcggcatggacgagctgtacaagtgatgaattgcggccgcctgacgtaactagtgtaaggcatgcaagcttgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcat

SEQ ID NO: 24 - DNA, 10423 bp, synthetic construct; identical to SEQ ID NO: 15
  source 1..10423: mol_type = other DNA
  misc_feature 1..10423: note = STOP(0) control (pHR-Tre3G-mCherry-STOP-OAZ1 FS-eYFP)

SEQ ID NO: 25 - DNA, 13023 bp, synthetic construct
  source 1..13023: mol_type = other DNA
  misc_feature 1..13023: note = ATP13A3 KO vector (pLenti-hU6-ATP13A3_sgRNA EF1-SpCas9-FLAG-P2A-Puro-WPRE)
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgtgggtgaaataacgaatctggtttaagagctatgctggaaacagcatagcaagtttaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac

SEQ ID NO: 26 - DNA, 13022 bp, synthetic construct
  source 1..13022: mol_type = other DNA
  misc_feature 1..13022: note = AAVS1 control (pLenti-hU6-AAVS1_sgRNA EF1-SpCas9-FLAG-P2A-Puro-WPRE)
gtcgacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagcgcgttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatcggcactgcgtgcgccaattctgcagacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggttaattaaggtaccgagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggggccactagggacaggatgtttaagagctatgctggaaacagcatagcaagtttaaataaggctagtccgttatcaacttgaaaaagtggcaccgagtcggtgcttttttgaattcgctagctaggtcttgaaaggagtgggaattggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggaccggttctagagcgctgccaccatggacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaagcgacctgccgccacaaagaaggctggacaggctaagaagaagaaagattacaaagacgatgacgataagggatccggcgcaacaaacttctctctgctgaaacaagccggagatgtcgaagagaatcctggaccgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggagtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaacgcgttaagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgtcgactttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgac